(19) United States
(12) Patent Application Publication
(10) Pub. No.: US 2025/0319165 A1
(43) Pub. Date: Oct. 16, 2025

(54) FEED ADDITIVE COMPRISING ENZYME COMBINATIONS

(71) Applicant: INTERNATIONAL N&H DENMARK APS, Kongens Lyngby (DK)

(72) Inventors: Shukun YU, Malmo (SE); Karsten KRAGH, Hoejbjerg (DK); Zhongmei TANG, SHANGHAI (CN); Zhenghong ZHANG, SHANGHAI (CN); Wenting LI, NEWARK (DE)

(21) Appl. No.: 18/864,872

(22) PCT Filed: May 16, 2023
(86) PCT No.: PCT/US2023/067061

§ 371 Date: Nov. 11, 2024

(30) Foreign Application Priority Data
May 17, 2022 (WO) PCT/CN2022/093295

(51) Int. Cl. A61K 38/47; A61K 31/05; A61K 31/11; A61K 31/205; A61K 35/00; A61K 35/741; A61K 35/742; A61K 35/744; A61P 1/14

(52) U.S. Cl. CPC A61K 38/47; A61K 31/05; A61K 31/11; A61K 31/205; A61K 35/741; A61K 35/742; A61K 35/744; A61P 1/14; C12Y 302/01001; C12Y 302/01003; A61K 2035/115

(57) ABSTRACT

Described herein, inter alia, are compositions and methods for improving starch digestibility and glucose yield in the small intestine of a ruminant animal.

### CROSS-REFERENCE TO RELATED APPLICATIONS

This application claims priority to International Patent Application No. PCT/CN2022/093295, filed May 17, 2022, the disclosure of which is incorporated by reference herein in its entirety.

### FIELD OF THE INVENTION

The field relates to animal nutrition and, in particular, to the use acid stable alpha-amylases in combination with glucoamylases as a feed additive for ruminants to enhance starch digestion and glucose yield in the small intestine.

### REFERENCE TO AN ELECTRONIC SEQUENCE LISTING

The contents of the electronic sequence listing (20230515_NB41951-WO-PCT2_Sequence_listing.xml; Size: 107,928 bytes; and Date of Creation: May 15, 2023) is herein incorporated by reference in its entirety.

### BACKGROUND

Ruminants have the unique ability to convert roughage into protein and energy through their microbial/enzyme digestive systems. Accordingly, ruminants play an important role in the earth's ecology and in the food chain.

The primary difference between ruminants and nonruminants is that ruminants' stomachs have four compartments: the rumen, reticulum, omasum, and abomasum. In the first two chambers, the rumen and the reticulum, the food is mixed with saliva and separates into layers of solid and liquid material. Solids clump together to form the cud or bolus.

The cud is then regurgitated and chewed to completely mix it with saliva and to break down the particle size. Fiber, especially cellulose and hemicellulose, is primarily broken down in these chambers by microbes (mostly bacteria, as well as some protozoa, fungi and yeast) into the three major volatile fatty acids (VFAs): acetic acid, propionic acid, and butyric acid. Protein and nonstructural carbohydrate (pectin, sugars, and starches) are also fermented.

Though the rumen and reticulum have different names, they represent the same functional space as digesta and can move back and forth between them. Together, these chambers are called the reticulorumen. The degraded digesta, which is now in the lower liquid part of the reticulorumen, then passes into the next chamber, the omasum, where water and many of the inorganic mineral elements are absorbed into the blood stream.

After this, the digesta is moved to the true stomach, the abomasum. The abomasum is the direct equivalent of the monogastric stomach, and digesta is digested here in much the same way. Digesta is finally moved into the small intestine, where the digestion and absorption of nutrients occurs. Microbes produced in the reticulorumen are also digested in the small intestine. Fermentation continues in the large intestine in the same way as in the reticulorumen.

Enzymes for use as feed additives ruminants are mainly fibrolytic enzymes, such as cellulases, beta-glucanases and hemicellulases (Table 1 in Beauchemin et al., 2004. Can. J. Anim. Sci. 84:23-36). Reports on starch hydrolases for ruminant uses are limited. Starch hydrolases are grouped as endo- and exo-amylases.

Accordingly, there is still a need to increase starch digestibility, increase glucose yield, particularly in the small intestine and/or increase digestion of dry matter in ruminants.

### SUMMARY

The present disclosure relates to compositions and methods for improving starch digestibility and glucose yield in the small intestine of ruminant animals via addition of one or more feed additives comprising at least one glucoamylase (EC 3.2.1.3) enzyme (for example a fungal glucoamylase enzyme) and at least one acid stable alpha-amylase (AsAA) enzyme (for example a fungal AsAA enzyme) to feed for the ruminant. Accordingly, in one aspect, provided herein are methods for increasing starch digestibility and glucose yield in in the small intestine of a ruminant animal comprising adding at least one glucoamylase (EC 3.2.1.3) enzyme and at least one acid stable alpha-amylase (AsAA) enzyme as a feed additive to feed for the ruminant, wherein said at least one glucoamylase and at least one AsAA has at least about 20% activity at pH less than or equal to about 3 in at least one of three digestive chambers of a ruminant comprising a rumen, an abomasum and/or a small intestine. In some embodiments, said at least one glucoamylase and at least one AsAA are capable of hydrolyzing raw starch under conditions comparable to those found in the rumen or abomasum. In some embodiments of any of the embodiments disclosed herein, the at least one AsAA is a member of glycoside hydrolase family 13 (GH 13) family or is a member of EC 3.2.1.1 or a variant or functional fragment thereof. In some embodiments of any of the embodiments disclosed herein, the at least one AsAA is at least about 60% identical to the amino acid sequence of SEQ ID NO: 3 or SEQ ID NO:4 or a variant or functional fragment thereof. In some embodiments of any of the embodiments disclosed herein, the at least one AsAA is at least about 60% identical to the amino acid sequence of SEQ ID NO: 6 or a variant or functional fragment thereof. In some embodiments, the at least one AsAA comprises at least one of SEQ ID NOs: 8-21 or a variant or functional fragment thereof. In some embodiments, the at least one AsAA comprises at least one of SEQ ID NOs: 22-73 or a variant or functional fragment thereof. In some embodiments of any of the embodiments disclosed herein, the at least one glucoamylase is at least about 60% identical to the glucoamylase of SEQ ID NO:5 or SEQ ID NO:7, or a variant or functional fragment thereof. In some embodiments of any of the embodiments disclosed herein, the ratio of glucoamylase to AsAA is about 70:30 to 96:4. In some embodiments, the ratio of glucoamylase to AsAA is about 96:4. In some embodiments of any of the embodiments disclosed herein, the at least one glucoamylase and/or at least one AsAA has at least about 20% activity at pH less than or equal to about 2.5. In some embodiments of any of the embodiments disclosed herein, the at least one glucoamylase and/or at least one AsAA has at least about 20% activity at pH less than or equal to about 3 for at least about 60 minutes. In some embodiments of any of the embodiments disclosed herein, the method further comprises adding at least one hemicellulase as a feed additive to the feed. In some embodiments of any of the embodiments disclosed herein, the method further comprises adding betaine as a feed additive to the feed. In some embodiments of any of the embodiments disclosed herein, the method further comprises adding at least one essential oil as a feed additive to the feed. In some embodiments, the essential oil comprises cinnamaldehyde and/or thymol. In some embodiments of any of the embodiments disclosed herein, the method further comprises adding at least one direct fed microbial (DFM) as a feed additive to the feed. In some embodiments, the direct fed microbial is a Megasphaera sp., Bacillus sp., a Propionibacterium sp., and/or an Enterococcus sp. In some embodiments of any of the embodiments disclosed herein, the ruminant is a beef cow, dairy cow, goat, sheep, giraffe, yak, deer, elk, antelope, water buffalo, or buffalo.

In other aspect, provided herein is a method for increasing milk production in a ruminant animal comprising adding at least one glucoamylase (EC 3.2.1.3) enzyme and at least one acid stable alpha-amylase (AsAA) enzyme as a feed additive to feed for the ruminant. In some embodiments, the at least one AsAA is a member of glycoside hydrolase family 13 (GH 13) family or is a member of EC 3.2.1.1 or a variant or functional fragment thereof. In some embodiments of any of the embodiments disclosed herein, the at least one AsAA is at least about 60% identical to the amino acid sequence of SEQ ID NO: 3 or SEQ ID NO:4 or a variant or functional fragment thereof. In some embodiments of any of the embodiments disclosed herein, the at least one AsAA is at least about 60% identical to the amino acid sequence of SEQ ID NO: 6 or a variant or functional fragment thereof. In some embodiments, the at least one AsAA comprises at least one of SEQ ID NOs: 8-21 or a variant or functional fragment thereof. In some embodiments, the at least one AsAA comprises at least one of SEQ ID NOs: 22-73 or a variant or functional fragment thereof. In some embodiments of any of the embodiments disclosed herein, the at least one glucoamylase is at least about 60% identical to the glucoamylase of SEQ ID NO: 5 or SEQ ID NO:7, or a variant or functional fragment thereof. In some embodiments of any of the embodiments disclosed herein, the ratio of glucoamylase to AsAA is about 70:30 to 96:4. In some embodiments, the ratio of glucoamylase to AsAA is about 96:4. In some embodiments of any of the embodiments disclosed herein, the at least one glucoamylase and/or at least one AsAA has at least about 20% activity at pH less than or equal to about 2.5. In some embodiments of any of the embodiments disclosed herein, the at least one glucoamylase and/or at least one AsAA has at least about 20% activity at pH less than or equal to about 3 for at least about 60 minutes. In some embodiments of any of the embodiments disclosed herein, the method further comprises adding at least one hemicellulase as a feed additive to the feed. In some embodiments of any of the embodiments disclosed herein, the method further comprises adding betaine as a feed additive to the feed. In some embodiments of any of the embodiments disclosed herein, the method further comprises adding at least one essential oil as a feed additive to the feed. In some embodiments, the essential oil comprises cinnamaldehyde and/or thymol. In some embodiments of any of the embodiments disclosed herein, the method further comprises adding at least one direct fed microbial (DFM) as a feed additive to the feed. In some embodiments, the direct fed microbial is a Megasphaera sp., Bacillus sp., a Propionibacterium sp., and/or an Enterococcus sp. In some embodiments of any of the embodiments disclosed herein, the ruminant is a beef cow, dairy cow, goat, sheep, giraffe, yak, deer, elk, antelope, water buffalo, or buffalo.

In a further aspect, provided herein is a feed additive composition comprising at least one glucoamylase (EC 3.2.1.3) enzyme and at least one acid stable alpha-amylase (AsAA) enzyme, wherein said at least one glucoamylase and at least one AsAA has at least about 20% activity at pH less than or equal to about 3 in at least one of three digestive chambers of a ruminant comprising a rumen, an abomasum and/or a small intestine. In some embodiments, said at least one glucoamylase and at least one AsAA are capable of hydrolyzing raw starch under conditions comparable to those found in the rumen or abomasum. In some embodiments of any of the embodiments disclosed herein, the at least one AsAA is a member of glycoside hydrolase family 13 (GH 13) family or is a member of EC 3.2.1.1 or a variant or functional fragment thereof. In some embodiments of any of the embodiments disclosed herein, the at least one AsAA is at least about 60% identical to the amino acid sequence of SEQ ID NO: 3 or SEQ ID NO: 4 or a variant or functional fragment thereof. In some embodiments of any of the embodiments disclosed herein, the at least one AsAA is at least about 60% identical to the amino acid sequence of SEQ ID NO:6 or a variant or functional fragment thereof. In some embodiments, the at least one AsAA comprises at least one of SEQ ID NOs: 8-21 or a variant or functional fragment thereof. In some embodiments, the at least one AsAA comprises at least one of SEQ ID NOs: 22-73 or a variant or functional fragment thereof. In some embodiments of any of the embodiments disclosed herein, the at least one glucoamylase is at least about 60% identical to the glucoamylase of SEQ ID NO:5 or SEQ ID NO:7, or a variant or functional fragment thereof. In some embodiments of any of the embodiments disclosed herein, the ratio of glucoamylase to AsAA is about 70:30 to 96:4. In some embodiments, the ratio of glucoamylase to AsAA is about 96:4. In some embodiments of any of the embodiments disclosed herein, the at least one glucoamylase and/or at least one AsAA has at least about 20% activity at pH less than or equal to about 2.5. In some embodiments of any of the embodiments disclosed herein, the at least one glucoamylase and/or at least one AsAA has at least about 20% activity at pH less than or equal to about 3 for at least about 60 minutes. In some embodiments of any of the embodiments disclosed herein, the composition further comprises at least one hemicellulase. In some embodiments of any of the embodiments disclosed herein, the composition further comprises betaine. In some embodiments of any of the embodiments disclosed herein, the composition further comprises at least one essential oil. In some embodiments, the essential oil comprises cinnamaldehyde and/or thymol. In some embodiments of any of the embodiments disclosed herein, the composition further comprises at least one direct fed microbial (DFM) as a feed additive to the feed. In some embodiments, the direct fed microbial is a Megasphaera sp., Bacillus sp., a Propionibacterium sp., and/or an Enterococcus sp.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### DETAILED DESCRIPTION

A ruminant is a mammal of the order Artiodactyla that digests plant-based food by initially softening it within the animal's first stomach chamber, then regurgitating the semi-digested mass, now known as cud, and chewing it again. The process of rechewing the cud to further break down plant matter and stimulate digestion is called “ruminating” or “rumination.”

Ruminants have a stomach with four chambers, namely the rumen, reticulum, omasum and abomasum. In the first two chambers, the rumen and the reticulum, food is mixed with saliva and separates into layers of solid and liquid material. Solids clump together to form the cud, or bolus. The cud is then regurgitated, chewed slowly to completely mix it with saliva, which further breaks down fibers. Fiber, especially cellulose, is broken down into glucose in these chambers by the enzymes produced by commensal bacteria, protozoa and fungi (such as cellulases, hemicellulases, amylases, phytases, and proteases). The broken-down fiber, which is now in the liquid part of the contents, then passes through the rumen and reticulum into the next stomach chamber, the omasum, where water is removed. The food in the abomasum is digested much like it would be in the human stomach. The abomasum has a pH of around 2.0 and therefore possesses an environment capable of denaturing most, if not all, polypeptides. The processed food is finally sent to the small intestine, where the absorption of the nutrients occurs.

Enzymes have been widely used for some time as additives in feed for monogastric animals to increase nutrient digestion and to reduce the environmental footprint of large-scale animal farming. Inclusion of phytases in feed has been one of the great success stories of this technology, with around 90% market penetration for monogastrics such as poultry and swine. In contrast, however, feed enzymes have seen very limited use as additives in ruminants despite intensive efforts (Meale et al., J. Anim. Sci. 2014. 92:427-442).

Numerous cellulases and hemicellulases have been tested in ruminants for dry matter intake, total tract dry matter digestion, and milk yield (Arriola et al., J. Dairy Sci. 2017. 100:4513-27) but the results showed high variation with no accompanying increase in feed efficacy. In ruminant nutrition, it is a challenge to bypass the rumen successfully to allow feed additives to reach the preferred site, which is often lower down the GI tract, e.g. the small intestine. Often the feed or feed additives used are degraded in the rumen environment (due to the presence of proteases produced by commensal microorganisms) or in the abomasum (due to the highly acidic environment) which results in either loss of form or activity of the feed additive. Therefore, larger quantities of feed additives are often used to compensate, thus adding to the costs of ruminant nutrition.

Despite these challenges, the inventors of the present application have surprisingly found that combinations of at least one glucoamylase enzyme and at least one acid stable alpha-amylase (AsAA) enzyme applied as feed additives to ruminant diets successfully improve starch digestion leading to improved weight gain to feed ratios and rib fat thickness in cattle. As described in the Examples section, these enzymes were administered to ruminant diets without the need of protective coatings and, despite the otherwise hostile rumen environment, still managed to effectuate improvements in digestive and growth parameters.

Accordingly, the inventors have discovered a means to ensure effective delivery of functional feed and feed additive enzymes to the small intestine of ruminant animals while avoiding substantial degradation in the rumen and abomasum.

### I. Definitions

Prior to describing the compositions and methods in detail, the following terms and abbreviations are defined.

Unless otherwise defined, all technical and scientific terms used have their ordinary meaning in the relevant scientific field. Singleton, et al., Dictionary of Microbiology and Molecular Biology, 2d Ed., John Wiley and Sons, New York (1994), and Halc & Markham, Harper Collins Dictionary of Biology, Harper Perennial, NY (1991) provide the ordinary meaning of many of the terms describing the invention.

The term “alpha-amylase” is used interchangeably with alpha-1,4-D-glucan glucanohydrolase and glycogenase. Alpha-amylases (E.C. 3.2.1.1) usually, but not always, need calcium in order to function. These enzymes catalyze the endohydrolysis of alpha-1,4-glucosidic linkages in oligosaccharides and polysaccharides. Alpha-amylases act on, starch, glycogen, and related polysaccharides and oligosaccharides in a random manner, liberating reducing groups in the alpha-configuration.

The term “acid-stable alpha amylase (“AsAA”) refers to an alpha amylase that is active in the pH range of pH 2.0 to 7.0 and such as 2.5 to 6.0. In some embodiments, an AsAA refers to an alpha-amylase that that has at least 20% activity at pH less than or equal to 3.0 compared its activity at pH 6.0.

“Glycoside hydrolase family 13” (GH13), as used herein, refers to a large sequence-based family of glycoside hydrolases containing a number of different enzyme activities and substrate specificities acting on α-glycosidic bonds (see Stam et al., 2006, Protein Eng Des Sel., (12):555-62).

The term “glucoamylase” (EC 3.2.1.3) is used interchangeably with glucan 1,4-alpha-glucosidase, amyloglucosidase, gamma-amylase, lysosomal alpha-glucosidase, acid maltase, exo-1,4-alpha-glucosidase, glucose amylase, gamma-1,4-glucan glucohydrolase, acid maltase, and 1,4-alpha-D-glucan hydrolase. These are exo-acting enzymes, which release glucosyl residues from the non-reducing ends of amylose and amylopectin molecules. The enzyme also hydrolyzes alpha-1,6 and alpha-1,3 linkages although at slower rates than alpha-1,4 linkages.

The term “enzyme variant,” as used herein, means a non-naturally occurring enzyme (such as an AsAA or a glucoamylase) having at least one (such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50) amino acid substitution(s) in a given parent enzyme amino acid sequence.

The term. “wild-type,” with respect to a polypeptide (such as an AsAA or a glucoamylase), refers to a naturally-occurring polypeptide that does not include a human-made substitution, insertion, or deletion at one or more amino acid positions.

The term “amino acid sequence” is synonymous with the terms “polypeptide”, “protein” and “peptide” and are used interchangeably. Where such amino acid sequences exhibit activity, they may be referred to as an “enzyme”. The conventional one-letter or three-letter codes for amino acid residues are used, with amino acid sequences being presented in the standard amino-to-carboxy terminal orientation (i.e., N→C).

The term “mature polypeptide” is defined herein as a polypeptide in its final form following translation and any post-translational modifications, such as N-terminal processing, C-terminal truncation, glycosylation, phosphorylation, etc. In one aspect, the predicted mature polypeptide is based on the analysis of SignalP software version 4.0 (Nordahl Petersen et al. (2011) Nature Methods, 8:785-786).

A “signal sequence” or “signal peptide” is a sequence of amino acids attached to the N-terminal portion of a protein, which facilitates the secretion of the protein outside the cell. The mature form of an extracellular protein lacks the signal sequence, which is cleaved off during the secretion process.

The term “nucleic acid” or “polynucleotide” can be used interchangeable to encompass DNA, RNA, heteroduplexes, and synthetic molecules capable of encoding a polypeptide. Nucleic acids may be single stranded or double stranded, and may be chemically modified. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

A “host strain” or “host cell” is an organism into which an expression vector, phage, virus, or other DNA construct, including a polynucleotide encoding a polypeptide of interest (e.g., an glucoamylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “expression” refers to the process by which a polypeptide is produced based on a nucleic acid sequence. The process includes both transcription and translation.

The term “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

The term “specific activity” refers to the number of moles of substrate that can be converted to product by an enzyme or enzyme preparation per unit time under specific conditions. Specific activity is generally expressed as units (U)/mg of protein or U/g of protein (such as glucoamylase (GA) or alpha-amylase (AsAA) units per mg or g of protein).

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, word length=12 to obtain nucleotide sequences homologous to a nucleic acid molecules of the present application. BLAST protein searches can be performed with the XBLAST program parameters set, e.g., to score-50, word length=3 to obtain amino acid sequences homologous to a protein molecule described herein. To obtain gapped alignments for comparison purposes. Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see. e.g., the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Another computer program that can be used to create multiple alignments of protein sequences is MUSCLE. Elements of the MUSCLE algorithm include fast distance estimation using kmer counting, progressive alignment using a new profile function described as log-expectation score, and refinement using tree-dependent restricted partitioning. This program is described in MUSCLE: multiple sequence alignment with high accuracy and high throughput by Robert C. Edgar (2004) published in Nucleic Acids Res. 32: 1792-1797. The percent identity between two sequences can be determined using techniques similar to those described above, with or without allowing gaps. In calculating percent identity, typically only exact matches are counted.

The term “recombinant” as used herein refers to an artificial combination of two otherwise separated segments of nucleic acid sequences, e.g., by chemical synthesis or by the manipulation of isolated segments of nucleic acids by genetic engineering techniques. For example, DNA in which one or more segments or genes have been inserted, either naturally or by laboratory manipulation, from a different molecule, from another part of the same molecule, or an artificial sequence, resulting in the introduction of a new sequence in a gene and subsequently in an organism. The terms “recombinant”, “transgenic”, “transformed”, “engineered” or “modified for exogenous gene expression” are used interchangeably herein.

The term “starch” is used interchangeably with “amylum”. It is a polymeric carbohydrate consisting of a large number of glucose units joined by glycosidic bonds and is the most common storage carbohydrate in plants. Thus. “starch” can refer to any material comprised of the complex polysaccharide carbohydrates of plants, comprised of amylose and amylopectin with the formula (C₆H₁₀O₅)ₓ, wherein X can be any number. In particular, the term refers to any plant-based material including but not limited to grains, grasses, tubers and roots and more specifically wheat, barley, corn, rye, rice, sorghum, brans, cassava, millet, potato, sweet potato, and tapioca.

The term “starch digestibility,” as used herein, refers to the complete or nearly complete (for example, any of about 83%, 84%, 85%, 86%, 87%, 88%, 89%, 90%, 91%, 92%, 93%, 94%, 95%, 96%, 97%, 98%, 99%, or 100% complete) conversion of starch polymer to glucose, for example, in the small intestine of a ruminant animal. Methods to ascertain starch digestibility can be found, for example, in Owens et al., 2016, Prof. Anim. Sci. 32:531-549, incorporated by reference herein.

The term “glucose yield.” as used herein, refers to the amount of glucose produced as a consequence of the digestion of starch, for example, in the small intestine of a ruminant animal. In another embodiment, glucose yield can refer to increasing the ratio of glucose to maltooligosaccharides in the range of about 1:1 to about 10:1 or greater than about 10:1 in a ruminant animal, for example, the small intestine of a ruminant animal. Without being bound to theory, increasing glucose yield in the small intestine can result in a reduced amount of maltooligosaccharides available for fermentation in the ilium and/or lower gastrointestinal tract of the ruminant animal.

The term “feed” is used with reference to products that are fed to animals in the rearing of livestock. The terms “feed” and “animal feed” and “feedstuff” are used interchangeably. In one embodiment, the food or feed is for consumption by non-ruminants and ruminants.

The term “fodder” as used herein refers to a type of animal feed, is any agricultural foodstuff used specifically to feed domesticated livestock, such as cattle, goats, sheep, horses, chickens and pigs. “Fodder” refers particularly to food given to the animals (including plants cut and carried to them), rather than that which they forage for themselves (called forage). Fodder is also called provender and includes hay, straw, silage, compressed and pelleted feeds, oils and mixed rations, and sprouted grains and legumes (such as bean sprouts, fresh malt, or spent malt). Most animal feed is from plants, but some manufacturers add ingredients to processed feeds that are of animal origin.

As used herein, “feed additive” refers to a substance that is added to animal feed for various purposes such as, without limitation, supplementing nutrition, preventing weight loss, enhancing digestion of fibers, and/or improving milk production. In some non-limiting embodiments, a feed additive can include one or more enzymes and/or betaine and/or one or more direct fed microbials and/or one or more essential oils.

A “premix,” as referred to herein, may be a composition composed of microingredients such as, but not limited to, one or more of vitamins, minerals, chemical preservatives, antibiotics, fermentation products, and other essential ingredients. Premixes are usually compositions suitable for blending into commercial rations.

The term “direct-fed microbial” (“DFM”) as used herein is source of live (viable) microorganisms that when applied in sufficient numbers can confer a benefit to the recipient thereof, i.e., a probiotic. A DFM can comprise one or more of such microorganisms such as bacterial strains. Categories of DFMs include, without limitation, Bacillus, Lactic Acid Bacteria, Megasphaera, Propionibacterium, Enterococcus, and Yeasts. Thus, the term DFM encompasses one or more of the following: direct fed bacteria, direct fed yeast, direct fed yeast and combinations thereof. Bacilli are unique, gram-positive rods that form spores. These spores are very stable and can withstand environmental conditions such as heat, moisture and a range of pH. These spores germinate into active vegetative cells when ingested by an animal and can be used in meal and pelleted diets. Lactic Acid Bacteria are gram-positive cocci that produce lactic acid which are antagonistic to pathogens. Since Lactic Acid Bacteria appear to be somewhat heat-sensitive, they are not used in pelleted diets. Types of Lactic Acid Bacteria include Bifidobacterium, Lactobacillus and Streptococcus.

The terms “probiotic,” “probiotic culture,” and “DFM” are used interchangeably herein and define live microorganisms (including bacteria or yeasts, for example) which, when for example ingested or locally applied in sufficient numbers, beneficially affects the host organism, i.e. by conferring one or more demonstrable health benefits on the host organism such as a health, digestive, and/or performance benefit. Probiotics may improve the microbial balance in one or more mucosal surfaces. For example, the mucosal surface may be the intestine, the urinary tract, the respiratory tract or the skin. The term “probiotic” as used herein also encompasses live microorganisms that can stimulate the beneficial branches of the immune system and at the same time decrease the inflammatory reactions in a mucosal surface, for example the gut. Whilst there are no lower or upper limits for probiotic intake, it has been suggested that at least 10⁶-10¹², for example at least 10⁶-10¹⁰, for example 10⁸-10⁹, cfu as a daily dose will be effective to achieve the beneficial health effects in a subject.

As used herein the term “betaine” refers to trimethylglycine. The compound is also called trimethylammonioacetate, 1-carboxy-N,N,N-trimethylmethanaminium, inner salt and glycine betaine. It is a naturally occurring quaternary ammonium type compound having the formula

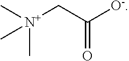

Betaine has a bipolar structure comprising a hydrophilic moiety (COO—) and a hydrophobic moiety (N+) capable of neutralizing both acid and alkaline solutions. In its pure form, betaine is a white crystalline compound that is readily soluble in water and lower alcohols. In the present invention betaine can be used, for example, as an anhydrous form, or as a hydrate or as an animal feed acceptable salt. In one embodiment, when betaine is present, it is present as the free zwitterion. In one embodiment, when betaine is present, it is present as anhydrous betaine. In one embodiment, when betaine is present, it is present as a monohydrate.

As used herein, “essential oil” refers to the set of all the compounds that can be distilled or extracted from a plant from which the oil is derived and that contributes to the characteristic aroma of that plant. See e.g., H. McGee, On Food and Cooking, Charles Scribner's Sons, p. 154-157 (1984). Non-limiting examples of essential oils include thymol and cinnamaldehyde.

As used herein, “effective amount” means a quantity of a substance (for example, an enzyme (such as an AsAA or a glucoamylase)), a direct fed microbial (DFM), or an essential oil (EO)) to improve one or more metrics in an animal. Improvement in one or more metrics of an animal (such as, without limitation, any of increased starch digestibility; improved milk production; improved feed conversion ratio (FCR); improved weight gain; improved feed efficiency; improved gut microbiome status (i.e. more healthy (“good”) bacterial and/or less unhealthy (“bad”) bacteria; and/or improved carcass quality can be measured as described herein or by other methods known in the art. An effective amount can be administered to the animal by providing ad libitum access to feed containing the substance. Additionally, substance can also be administered in one or more doses.

The term “ruminant” as used herein refers to a mammal that is able to acquire nutrients from plant-based food by fermenting it in a specialized stomach prior to digestion, principally, through microbial actions. The process typically requires the fermented ingesta (known as cud) to be regurgitated and chewed again. The process of rechewing the cud to further break down plant matter and stimulate digestion is called rumination. Roughly 150 species of ruminants include both domestic and wild species. Ruminating animals include, but are not limited to, cattle, cows, goats, sheep, giraffes, yaks, deer, elk, antelope, buffalo and the like.

The term “digestive chambers of a ruminant” as used herein refer to the rumen, reticulum, omasum, abomasum and small intestine (McDonald et al., 2011, Animal Nutrition (7th Edition), pages 156-191). The abomasum is the direct equivalent of the monogastric stomach.

As used herein, the term “rumen environment” refers to the conditions within the rumen. In general, the rumen has a temperature of about 39° C., and a pH in the range of 5 to 7 and is colonized by microbes. As the environment inside a rumen is anaerobic, most microbial species are obligate or facultative anaerobes that can decompose complex plant material, such as cellulose, hemicellulose, starch, and proteins. The hydrolysis of cellulose results in sugars, which are further fermented to products such as acetate, lactate, propionate, butyrate, carbon dioxide and methane. In one embodiment, degradation of exogenously fed enzymes is primarily due to the action of rumen microbes present in the rumen environment. In some embodiments, reaction conditions in 0.1M MES buffer at pH 6.0 simulates the rumen environment. In other embodiments. “rumen environment” can refer generally to the entire upper gastrointestinal tract of ruminant animals which includes the rumen, reticulum, omasum and abomasum.

As used herein with regard to amino acid residue positions, “corresponding to” or “corresponds to” or “correspond to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related protein or a reference protein.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −15% to +15% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

The term “comprising” and its cognates are used in their inclusive sense; that is, equivalent to the term “including” and its corresponding cognates. It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Compositions

In a first aspect, the present invention relates to feed additive compositions comprising at least one glucoamylase (EC 3.2.1.3) enzyme (for example, a fungal glucoamylase enzyme) and at least one acid stable alpha-amylase (AsAA) enzyme (for example, a fungal AsAA enzyme).

### A. Acid Stable Alpha-Amylases (AsAAs)

Alpha amylases for use in the compositions and methods disclosed herein can be a wild-type alpha amylase, a variant or fragment thereof or a hybrid alpha amylase which is derived from for example a catalytic domain from one microbial source and a starch binding domain from another microbial source. Alternatively, the alpha amylase can be a variant that has been engineered to be acid stable.

Non-limiting examples of fungal alpha amylases suitable for use in the methods and compositions disclosed herein include those obtained from fungal and filamentous fungal strains including, but not limited to, strains of Aspergillus (e.g., A. niger, A. kawachi, A. oryzae, A. welwitschiae, A. brasiliensis, A. sclerotiicarbonarius, A. felis, A. hiratsukae, A. viridinutans, A. sclerotialis, A. leporis, A. tanneri, A. avenaceus, A. novofumigatus, A. nomiae, A. flavus, A. udagawae, A. ellipticus, A. puulaavensis, A. nidulans, A. welwitschiae, A. brasiliensis, and A. scleroniicarbonarius), Trichoderma sp., Rhizopus sp., Mucor sp., Penicillium sp., (e.g., P. rolfsii, P. italicum, and P. steckii), Acidomyces sp. (e.g., A. richmondensis), Bispora sp., Symbiotaphrina sp., (e.g. S. kochii), Loramyces sp., (e.g. L. juncicola), Kockovaella sp. (e.g., K. imperatae). Saitozyma sp. (e.g. S. flava), Cryptococcus sp., Saitozyma sp. (e.g. S. flava). Talaromyces sp. (e.g., T. rugulosus, T. amestolkiae, T. crustaceus, and T. atroroseus). In some embodiments, the alpha amylase is obtained from a strain of Aspergillus niger (AniAmyl) or a strain of Acidomyces richmondensis (AriAmy1).

In one embodiment, the AsAA comprises an amino acid sequence having at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% at least about 99% or at least about 100% sequence identity with the amino acid sequence set forth in SEQ ID NO: 3 or SEQ ID NO:4. The AsAA can comprise an amino acid sequence at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% at least about 99% or at least about 100% sequence identity with the amino acid sequence set forth in any of SEQ ID NO:8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 21. In some embodiments, the AsAA has a predicted mature amino acid sequence of SEQ ID NO: 4. In other embodiments, the AsAA has a predicted mature amino acid sequence from amino acid position 15-634 of SEQ ID NO:3. In other embodiments, the AsAA has a predicted mature amino acid sequence from amino acid position 16-634 of SEQ ID NO:3. In other embodiments, the AsAA has a predicted mature amino acid sequence from amino acid position 17-634 of SEQ ID NO:3. In other embodiments, the AsAA has a predicted mature amino acid sequence from amino acid position 18-634 of SEQ ID NO:3. In other embodiments, the AsAA has a predicted mature amino acid sequence from amino acid position 19-634 of SEQ ID NO: 3. In other embodiments, the AsAA has a predicted mature amino acid sequence from amino acid position 20-634 of SEQ ID NO:3. In other embodiments, the AsAA has a predicted mature amino acid sequence from amino acid position 21-634 of SEQ ID NO:3. In other embodiments, the AsAA has a predicted mature amino acid sequence from amino acid position 22-634 of SEQ ID NO:3. In other embodiments, the AsAA has a predicted mature amino acid sequence from amino acid position 23-634 of SEQ ID NO:3. In other embodiments, the AsAA has a predicted mature amino acid sequence from amino acid position 24-634 of SEQ ID NO:3. In other embodiments, the AsAA has a predicted mature amino acid sequence from amino acid position 25-634 of SEQ ID NO:3. In another embodiment, the AsAA comprises an amino acid sequence having at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% at least about 99% or at least about 100% sequence identity with the amino acid sequence set forth in SEQ ID NO: 6. The AsAA can comprise an amino acid sequence at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity with the amino acid sequence set forth in any of SEQ ID NO:22. SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51. SEQ ID NO: 52. SEQ ID NO: 53. SEQ ID NO: 54. SEQ ID NO: 55. SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73.

The alpha-amylases for use in the methods and compositions disclosed herein (such as any of the alpha amylases of SEQ ID NOs: 3-4, 6, or 8-73) are acid stable alpha amylases which, when added in an effective amount, have and/or maintain enzymatic activity (such as at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% enzymatic activity) in the pH range of about 2.0 to about 7.0, such as at a pH less than or equal to about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In addition, the acid stable alpha-amylases for use in the methods and compositions disclosed herein (such as any of the alpha amylases of SEQ ID NOs: 3-4, 6, or 8-73) maintain enzymatic activity (such as at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% enzymatic activity) following exposure to a low pH environment (such as the ruminal environment having a pH of about 3, 2.9, 2.8, 2.7, 2.6, 2.5, 2.4, 2.3, 2.2, 2.1, or 2 or less) for any of about 30, 35, 40, 45, 50, 55, 60, 65, 70, 75, 80, 85, 90, 95, 100, 105, 110, 115, 120, 125, 130, 135, 140, 145, 150, 155, 160, 165, 170, 175 or 180 minutes.

### B. Glucoamylases

Glucoamylase (1,4-alpha-D-glucan glucohydrolase, EC 3.2.1.3) is an enzyme, which catalyzes the release of D-glucose from the non-reducing ends of starch or related oligo- and poly-saccharide molecules. Glucoamylases are produced by several filamentous fungi and yeast.

In one embodiment, provided herein are feed or feed additive compositions including one or more glucoamylase. The glucoamylase may be any commercially available glucoamylase. Suitably the glucoamylase may be an 1,4-alpha-D-glucan glucohydrolase (EC 3.2.1.3). All E.C. enzyme classifications referred to herein relate to the classifications provided in Enzyme Nomenclature—Recommendations (1992) of the nomenclature committee of the International Union of Biochemistry and Molecular Biology—ISBN 0-12-226164-3, which is incorporated herein.

Glucoamylases have been used successfully in commercial applications for many years. Additionally, various mutations have been introduced in fungal glucoamylases, for example, Trichoderma reesei glucoamylase (TrGA), to enhance thermal stability and specific activity. See, e.g., WO 2008/045489; WO 2009/048487; WO 2009/048488; and U.S. Pat. No. 8,058,033. In some embodiments, the T. reesei glucoamylase (TrGA) is PDB accession number is 2VN4_A or is SEQ ID NO: 11 from WO2019/173424, incorporated by reference herein. Glucoamylase activity can be assessed using any means known in the art, including those described in the Examples section, infra.

A glucoamylase may be derived from any suitable source, e.g., derived from a microorganism or a plant. Glucoamylases can be from fungal or bacterial origin, selected from the group consisting of Aspergillus glucoamylases, in for example, Aspergillus niger G1 or G2 glucoamylase (Boel et al., 1984, EMBO J. 3 (5): 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Hata et al., 1991, Agric. Biol. Chem. 55(4): 941-949), or variants or fragments thereof. Other Aspergillus glucoamylase variants include variants with enhanced thermal stability: G137A and G139A (Chen et al., 1996, Prot. Eng. 9:499-505); D257E and D293E/Q (Chen et al., 1995, Prot. Eng. 8:575-582); N182 (Chen et al., 1994, Biochem. J. 301:275-281); disulphide bonds, A246C (Fierobe et al., 1996, Biochemistry 35:8698-8704; and introduction of Pro residues in positions A435 and S436 (Li et al., 1997, Protein Eng. 10:1199-1204. In some embodiments, the A. niger glucoamylase (AnGA) is NCBI accession number XP 001390530.1 or is SEQ ID NO: 10 from WO2019/173424, incorporated by reference herein. In other embodiments, the glucoamylase is from Aspergillus fumigatus and is SEQ ID NO:4 from WO2017112635, incorporated by reference herein.

Other glucoamylases include Athelia rolfsii (previously denoted Corticium rolfsi) glucoamylase (see U.S. Pat. No. 4,727,026 and Nagasaka et al., 1998, Appl. Microbiol. Biotechnol. 50:323-330), Talaromyces glucoamylases, in particular derived from Talaromyces duponti, Talaromyces emersonii (WO 99/28448), Talaromyces leycettanus (U.S. Pat. No. Re. 32,153), and Talaromyces thermophilus (U.S. Pat. No. 4,587,215). In some embodiments, the glucoamylase is from Wolfiporia cocos having an NCBI access ion number PCH39892.1 or is SEQ ID NO: 8 from WO2019/173424, incorporated by reference herein.

Bacterial glucoamylases include glucoamylases from Clostridium, in particular C. thermoamylolyticum (EP 135138) and C. thermohydrosulfuricum (WO86/01831), Trametes cingulata, Pachykytospora papyracea, and Leucopaxillus giganteus, all disclosed in WO 2006/069289; or Peniophora rufomarginata disclosed in WO2007/124285 or PCT/US2007/066618; or a mixture thereof. A hybrid glucoamylase may be used in the present invention. Examples of hybrid glucoamylases are disclosed in WO 2005/045018. Specific examples include the hybrid glucoamylase disclosed in Tables 1 and 4 of Example 1 (which hybrids are hereby incorporated by reference).

Commercially available glucoamylase compositions include AMG 200L; AMG 300L; SAN™ SUPER, SAN™ EXTRA L, SPIRIZYME™ PLUS, SPIRIZYME™ FUEL, SPIRIZYME™ B4U, SPIRIZYME ULTRA, SPIRIZYME™ EXCEL and AMG™ E (from Novozymes A/S, Denmark); OPTIDEX™ 300, GC480™ and GC147™ (from Danisco US, Inc.); AMIGASE™ and AMIGASE™ PLUS (from DSM); G-ZYME™ G900, G-ZYME™ and G990 ZR (from Danisco US, Inc.).

The glucoamylase can comprise an amino acid sequence at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity with the amino acid sequence set forth in SEQ ID NO:5 or SEQ ID NO:7.

In some embodiments, the glucoamylases for use in the methods and compositions disclosed herein (such as any of the glucoamylases of SEQ ID NOs: 5 or SEQ ID NO:7) are acid stable glucoamylases which, when added in an effective amount, have and/or maintain enzymatic activity (such as at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% enzymatic activity) in the pH range of about 2.0 to about 7.0, such as at a pH less than or equal to about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In addition, the glucoamylases for use in the methods and compositions disclosed herein (such as any of the glucoamylases of SEQ ID NOs: 5 or SEQ ID NO:7) maintain enzymatic activity (such as at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% enzymatic activity) following exposure to a low pH environment (such as the ruminal environment having a pH of about 2) for any of about 30, 35, 40, 45, 50, 55, 60, 65, 70, 75, 80, 85, 90, 95, 100, 105, 110, 115, 120, 125, 130, 135, 140, 145, 150, 155, 160, 165, 170, 175 or 180 minutes.

### C. Feed Additive Compositions

In one embodiment, provided herein are feed additive and/or feed additive compositions comprising one or more of the glucoamylases and AsAAs disclosed herein.

In one embodiment, the feed additive composition may be used in the form of solid or liquid preparations or alternatives thereof. Examples of solid preparations include powders, pastes, boluses, capsules, ovules, pills, pellets, tablets, dusts, and granules which may be wettable, spray-dried or freeze-dried. Examples of liquid preparations include, but are not limited to, aqueous, organic or aqueous-organic solutions, suspensions and emulsions.

In another embodiment, the feed additive composition can be used in a solid form. In one embodiment, the solid form is a pelleted form. In solid form, the feed additive composition may also contain one or more of: excipients such as microcrystalline cellulose, lactose, sodium citrate, calcium carbonate, dibasic calcium phosphate and glycine; disintegrants such as starch (In some embodiments, corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

In one embodiment, the feed additive composition is formulated to a dry powder or granules as described in WO2007/044968 (referred to as TPT granules) or WO 1997/016076 or WO 1992/012645 (each of which is incorporated herein by reference).

In one embodiment, the feed additive composition may be formulated to a granule feed composition comprising: one or more glucoamylases and one or more AsAAs. In one embodiment, the one or more glucoamylases and one or more AsAAs retains activity after processing. In one embodiment, the one or more glucoamylases and one or more AsAAs retains an activity level after processing selected from the group consisting of: 50-60% activity. 60-70% activity, 70-80% activity, 80-85% activity, 85-90% activity, and 90-95% activity.

In yet another embodiment, a granule containing one or more of the one or more glucoamylases and one or more AsAAs may be produced using a feed pelleting process and the feed pretreatment process may be conducted between 70° C., and 95° C., for up to several minutes, such as between 85° C., and 95° C. In another embodiment, the granule may be produced using a steam-heated pelleting process that may be conducted between 85° C., and 95° C., for up to several minutes.

In one embodiment, the granule may have a moisture barrier coating selected from polymers and gums and the moisture hydrating material may be an inorganic salt. The moisture hydrating coating may be between 25% and 45% w/w of the granule and the moisture barrier coating may be between 2% and 20% w/w of the granule.

In one embodiment, the one or more glucoamylases and one or more AsAAs retains activity after conditions selected from one or more of: (a) a feed pelleting process; (b) a steam-heated feed pretreatment process; (c) storage; (d) storage as an ingredient in an unpelleted mixture; and (c) storage as an ingredient in a feed base mix or a feed premix comprising at least one compound selected from trace minerals, organic acids, reducing sugars, vitamins, choline chloride, and compounds which result in an acidic or a basic feed base mix or feed premix.

In some embodiments, the feed additive compositions may be diluted using a diluent, such as starch powder, lime stone or the like. In one embodiment, the one or more glucoamylases and one or more AsAAs may be in a liquid formulation suitable for consumption. In some embodiments, such liquid consumption contains one or more of the following: a buffer, salt, sorbitol and/or glycerol. In another embodiment, the feed additive composition may be formulated by applying, e.g. spraying, the enzyme(s) onto a carrier substrate, such as ground wheat for example.

In one embodiment, the feed additive composition may be formulated as a premix. By way of example only, the premix may comprise one or more feed components, such as one or more minerals and/or one or more vitamins.

In another embodiment, the feed additive composition can be delivered as an aqueous suspension and/or an elixir. The feed additive composition may be combined with various sweetening or flavoring agents, coloring matter or dyes, with emulsifying and/or suspending agents and with diluents such as water, propylene glycol and glycerin, and combinations thereof.

In some the ratio of glucoamylase to AsAA for use in the compositions and methods disclosed herein is from about 70:30 to 96:2, such as any of about 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, or 98:2.

In some embodiments, the AsAA is added to the feed additive composition or feed for use in any of the methods or compositions disclosed herein in a dose of about 0.1 g to about 50 g per ton treated feed, such as about 1 g to about 15 g per ton of treated feed, such as any of about 0.1 g, 0.2 g, 0.3 g, 0.4 g, 0.5 g, 0.6 g, 0.7 g, 0.8 g, 0.9 g, 1 g, 1.5 g, 2 g, 2.5 g, 3 g, 3.5 g, 4 g, 4.5 g, 5 g, 5.5 g, 6 g, 6.5 g, 7 g, 7.5 g, 8 g, 8.5 g, 9 g, 9.5 g, 10 g, 10.5 g, 11 g, 11.5 g, 12 g, 12.5 g, 13 g, 13.5 g, 14 g, 14.5 g, 15 g, 16 g, 17 g, 18 g, 19 g, 20 g, 21 g, 22 g, 23 g, 24 g, 25 g, 26 g, 27 g, 28 g, 29 g, 30 g, 31 g, 32 g, 33 g, 34 g, 35 g, 36 g, 37 g, 38 g, 39 g, 40 g, 41 g, 42 g, 43 g, 44 g, 45 g, 46 g, 47 g, 48 g, 49 g, or 50 g per ton of treated feed. In another embodiment, the AsAA is added to the feed additive composition or feed for use in any of the methods or compositions disclosed herein in a dose of about 0.25-250 mg per kg feed, such as any of about 0.25 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, or 250 mg per kg feed, inclusive of all amounts falling in between these values. In still other embodiments, the AsAA is added to the feed additive composition or feed for use in any of the methods or compositions disclosed herein in a dose of about 0.1-100 alpha-amylase (AA) units per kg feed, where AA units per kg feed is calculated based on the method described in Example 1.

In other embodiments, the glucoamylase is added to the feed additive composition or feed for use in any of the methods or compositions disclosed herein in a dose of about 0.1 g to about 50 g per ton treated feed, such as about 1 g to about 15 g per ton of treated feed, such as any of about 0.1 g, 0.2 g, 0.3 g, 0.4 g, 0.5 g, 0.6 g, 0.7 g, 0.8 g, 0.9 g, 1 g, 1.5 g, 2 g, 2.5 g, 3 g, 3.5 g, 4 g, 4.5 g, 5 g, 5.5 g, 6 g, 6.5 g, 7 g, 7.5 g, 8 g, 8.5 g, 9 g, 9.5 g, 10 g, 10.5 g, 11 g, 11.5 g, 12 g, 12.5 g, 13 g, 13.5 g, 14 g, 14.5 g, 15 g, 16 g, 17 g, 18 g, 19 g, 20 g, 21 g, 22 g, 23 g, 24 g, 25 g, 26 g, 27 g, 28 g, 29 g, 30 g, 31 g, 32 g, 33 g, 34 g, 35 g, 36 g, 37 g, 38 g, 39 g, 40 g, 41 g, 42 g, 43 g, 44 g, 45 g, 46 g, 47 g, 48 g, 49 g. or 50 g per ton of treated feed. In another embodiment, the glucoamylase is added to the feed additive composition or feed for use in any of the methods or compositions disclosed herein in a dose of about 5-5000 mg per kg feed, such as any of about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1500 mg, 2000 mg, 2500 mg, 3000 mg, 3500 mg, 4000 mg, 4500 mg, or 5000 mg per kg feed, inclusive of all amounts falling in between these values. In still other embodiments, the glucoamylase is added to the feed additive composition or feed for use in any of the methods or compositions disclosed herein in a dose of about 1-1000 glucoamylase (GA) units per kg feed, where GA units per kg feed is calculated based on the method described in Example 1.

### D. Feedstuffs

In another embodiment, provided herein are feed additive and/or feed additive compositions containing any of the glucoamylase and AsAA compositions disclosed herein that may be used as a feed or in the preparation of a feed. The feed may be in the form of a solution or as a solid depending on the use and/or the mode of application and/or the mode of administration. When used as a feed or in the preparation of a feed, such as functional feed, the feed additive composition may be used in conjunction with one or more of the following: a nutritionally acceptable carrier, a nutritionally acceptable diluent, a nutritionally acceptable excipient, a nutritionally acceptable adjuvant, a nutritionally active ingredient.

In one embodiment, the feed additive composition disclosed herein is admixed with a feed component to form a feedstuff. In one embodiment, the feed may be a fodder, or a premix thereof, a compound feed, or a premix thereof. In one embodiment, the feed additive composition disclosed herein may be admixed with a compound feed, a compound feed component or a premix of a compound feed or to a fodder, a fodder component, or a premix of a fodder.

In one embodiment, fodder may be obtained from one or more of the plants selected from: alfalfa (lucerne), barley, birdsfoot trefoil, brassicas, Chau moellier, kale, rapeseed (canola), rutabaga (swede), turnip, clover, alsike clover, red clover, subterranean clover, white clover, grass, false oat grass, fescue, Bermuda grass, brome, heath grass, meadow grasses (from naturally mixed grassland swards, orchard grass, rye grass, Timothy-grass, corn (maize), millet, oats, sorghum, soybeans, trees (pollard tree shoots for tree-hay), wheat, and legumes.

Compound feeds can be complete feeds that provide all the daily required nutrients, concentrates that provide a part of the ration (protein, energy) or supplements that only provide additional micronutrients, such as minerals and vitamins. The main ingredients used in compound feed are the feed grains, which include corn, soybeans, sorghum, oats, and barley.

In one embodiment, a feedstuff as disclosed herein may comprise one or more feed materials selected from the group comprising cereals, such as small grains (e.g., wheat, barley, rye, oats and combinations thereof) and/or large grains such as maize or sorghum; by products from cereals, such as corn gluten meal, Distillers Dried Grain Solubles (DDGS), wheat bran, wheat middlings, wheat shorts, rice bran, rice hulls, oat hulls, palm kernel, and citrus pulp; protein obtained from sources such as soya, sunflower, peanut, lupin, peas, fava beans, cotton, canola, fish meal, dried plasma protein, meat and bone meal, potato protein, whey, copra, sesame; oils and fats obtained from vegetable and animal sources; and minerals and vitamins.

In yet another embodiment, a feedstuff may comprise at least one high fiber feed material and/or at least one by-product of the at least one high fiber feed material to provide a high fiber feedstuff. Examples of high fiber feed materials include: wheat, barley, rye, oats, by products from cereals, such as corn gluten meal. Distillers Dried Grain Solubles (DDGS), wheat bran, wheat middlings, wheat shorts, rice bran, rice hulls, oat hulls, palm kernel, and citrus pulp. Some protein sources may also be regarded as high fiber: protein obtained from sources such as sunflower, lupin, fava beans and cotton.

In still another embodiment, the feed may be one or more of the following: a compound feed and premix, including pellets, nuts or (cattle) cake; a crop or crop residue: corn, soybeans, sorghum, oats, barley, corn stover, copra, straw, chaff, sugar beet waste; fish meal; freshly cut grass and other forage plants; meat and bone meal; molasses; oil cake and press cake; oligosaccharides; conserved forage plants: hay and silage; seaweed; seeds and grains, either whole or prepared by crushing, milling etc.; sprouted grains and legumes; yeast extract.

In one embodiment, the feed additive composition of disclosed herein is admixed with the product (e.g. feedstuff). Alternatively, the feed additive composition may be included in the emulsion or raw ingredients of a feedstuff. In another embodiment, the feed additive composition is made available on or to the surface of a product to be affected/treated. In still another embodiment, the feed additive compositions disclosed herein may be applied, interspersed, coated and/or impregnated to a product (e.g. feedstuff or raw ingredients of a feedstuff) with a controlled amount of one or more glucoamylases and one or more AsAAs.

### E. Additional Active Agents

In further embodiments, any of the feed additive and/or feed additive compositions disclosed herein can contain one or more additional active agents.

As used herein, the term “active agent” can be any material that is to be added to a feed additive and/or feed additive composition (such as a glucoamylase and AsAA-containing feed additive composition) to provide the intended functionality for a given use. The active agent may be a biologically viable material, a food or feed ingredient, an antimicrobial agent, an antibiotic replacement agent, a prebiotic, a probiotic, an agrochemical ingredient, such as a pesticide, fertilizer or herbicide; a pharmaceutical ingredient or a household care active ingredient, or combinations thereof.

In a one embodiment, the active agent is a protein, enzyme, peptide, polypeptide, amino acid, carbohydrate, lipid or oil, vitamin, co-vitamin, hormone, or combinations thereof. Inherently thermostable active agents are encompassed by the present teachings and can exhibit enhanced thermostability to the components of the feed additive and/or feed additive composition. Some non-limiting active agents for food and feed applications are enzymes, peptides and polypeptides, amino acids, antimicrobials, gut health promoting agents, vitamins, and combinations thereof.

### 1. Additional Enzymes

Additional enzymes can be included in the glucoamylase and AsAA-containing feed additive compositions disclosed herein. Any enzyme may be used, and a nonlimiting list of enzymes include phytases, xylanases, 3-glucanases, phosphatases, proteases, additional amylases and/or glucoamylases, pullulanases, cellulases, lipases, cutinases, oxidases, transferases, reductases, glucoamylases, hemicellulases, mannanases, esterases, isomerases, pectinases, lactases, peroxidases, laccases, other redox enzymes and mixtures thereof. The above enzyme lists are examples only and are not meant to be exclusive. Any enzyme may be used in the compositions and methods of the present invention, including wild type, recombinant and variant enzymes of bacterial, fungal, yeast, plant, insect and animal sources, and acid, neutral or alkaline enzymes. It will be recognized by those skilled in the art that the amount of enzyme used will depend, at least in part, upon the type and property of the selected enzyme and the intended use.

In another embodiment, one or more additional enzyme for inclusion as an additional active agent in the feed additive compositions disclosed herein is one or more hemicellulase. As used herein, a “hemicellulase” is any polypeptide which is capable of degrading or modifying hemicellulose. That is to say, a hemicellulase may be capable of degrading or modifying one or more of xylan, glucuronoxylan, arabinoxylan, glucomannan and xyloglucan. A polypeptide which is capable of degrading a hemicellulose is one which is capable of catalyzing the process of breaking down the hemicellulose into smaller polysaccharides, either partially, for example into oligosaccharides, or completely into sugar monomers, for example hexose or pentose sugar monomers. A hemicellulase as described herein may give rise to a mixed population of oligosaccharides and sugar monomers. Such degradation will typically take place by way of a hydrolysis reaction. Depending on the catalytic mechanism, these enzymes have been referred to as xylanase, arabinoxylanase, beta-glucanase, beta-mannanase, pectinase, arabinase, pectin methylesterase, pectin lyase, and polygalacturonases, and the like.

### 2. Direct Fed Microbials (DFMs)

In one embodiment, a DFM can be included as an active agent in the glucoamylase and AsAA-containing feed additive compositions disclosed herein and, optionally, may be formulated as a liquid, a dry powder or a granule. In one embodiment, the DFMs and glucoamylase and AsAA-containing feed additive compositions disclosed herein can be formulated as a single mixture. In another embodiment, the DFMs and glucoamylase and AsAA-containing feed additive compositions can be formulated as separate mixtures. In still another embodiment, separate mixtures of DFMs and glucoamylase and AsAA-containing feed additive compositions can be administered at the same time or at different times. In still another embodiment, separate mixtures of DFMs and glucoamylase and AsAA-containing feed additive compositions can be administered simultaneously or sequentially. In yet another embodiment, a first mixture comprising DFMs can be administered followed by a second mixture comprising glucoamylase and AsAA-containing feed additive compositions. In still another embodiment, a first mixture comprising glucoamylase and AsAA-containing feed additive compositions can be administered followed by a second mixture comprising DFMs.

Dry powder or granules may be prepared by means known to those skilled in the art, such as, in top-spray fluid bed coater, in a bottom spray Wurster or by drum granulation (e.g. High sheer granulation), extrusion, pan coating or in a microingredients mixer.

In another embodiment, the glucoamylase and AsAA, additional enzymes, and/or DFMs may be coated, for example encapsulated. Suitably the glucoamylase and AsAA, additional enzymes, and/or DFMs may be formulated within the same coating or encapsulated within the same capsule. Alternatively, one or more of the additional enzymes and/or DFMs may be formulated within the same coating or encapsulated within the same capsule while the glucoamylase and AsAA can be formulated in a separate coating.

In some embodiments, such as where the DFM is capable of producing endospores, the DFM may be provided without any coating. In such circumstances, the DFM endospores may be simply admixed with glucoamylase and AsAA-containing feed additive composition. The glucoamylase and AsAA, enzymes, and/or DFMs may be encapsulated as mixtures (i.e. comprising one or more, two or more, three or more or all) or they may be encapsulated separately, e.g. singly.

At least one DFM may comprise at least one viable microorganism such as a viable bacterial strain or a viable yeast or a viable fungi. In some embodiments, the DFM comprises at least one viable bacteria. It is possible that the DFM may be a spore forming bacterial strain and hence the term DFM may be comprised of or contain spores, e.g. bacterial spores. Thus, the term “viable microorganism” as used herein may include microbial spores, such as endospores or conidia. Alternatively, the DFM in the feed additive composition described herein may not comprise of or may not contain microbial spores, e.g. endospores or conidia. The microorganism may be a naturally-occurring microorganism or it may be a transformed microorganism.

A DFM as described herein may comprise microorganisms from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera and combinations thereof. In some embodiments, the DFM comprises one or more bacterial strains selected from the following Bacillus spp: Bacillus subtilis, Bacillus cereus, Bacillus licheniformis, Bacillus pumilis and Bacillus amyloliquefaciens.

The genus “Bacillus”, as used herein, includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. gibsonii, B. pumilis and B. thuringiensis. It is recognized that the genus Bacillus continues to undergo taxonomical reorganization. Thus, it is intended that the genus include species that have been reclassified, including but not limited to such organisms as Bacillus stearothermophilus, which is now named “Geobacillus stearothermophilus”, or Bacillus polymyxa, which is now “Paenibacillus polymyxa” The production of resistant endospores under stressful environmental conditions is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In another aspect, the DFM may be further combined with the following Lactococcus spp: Lactococcus cremoris and Lactococcus lactis and combinations thereof. The DFM may be further combined with the following Lactobacillus spp: Lactobacillus buchneri, Lactobacillus acidophilus, Lactobacillus casei, Lactobacillus kefiri, Lactobacillus bifidus, Lactobacillus brevis, Lactobacillus helveticus, Lactobacillus paracasei, Lactobacillus rhamnosus, Lactobacillus salivarius, Lactobacillus curvatus, Lactobacillus bulgaricus, Lactobacillus sakei, Lactobacillus reuteri, Lactobacillus fermentum, Lactobacillus farciminis, Lactobacillus lactis, Lactobacillus delbreuckii, Lactobacillus plantarum, Lactobacillus paraplantarum, Lactobacillus farciminis, Lactobacillus rhamnosus, Lactobacillus crispatus, Lactobacillus gasseri, Lactobacillus johnsonii and Lactobacillus jensenii, and combinations of any thereof.

In still another aspect, the DFM may be further combined with the following Bifidobacteria spp: Bifidobacterium lactis, Bifidobacterium bifidium, Bifidobacterium longum, Bifidobacterium animalis, Bifidobacterium breve, Bifidobacterium infantis, Bifidobacterium catenulatum, Bifidobacterium pseudocatenulatum, Bifidobacterium adolescentis, and Bifidobacterium angulatum, and combinations of any thereof.

There can be mentioned bacteria of the following species: Bacillus subtilis, Bacillus licheniformis, Bacillus amyloliquefaciens, Bacillus pumilis, Enterococcus, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Bacillus subtilis, Propionibacterium thoenii, Lactobacillus farciminis, Lactobacillus rhamnosus, Megasphaera elsdenii (such as any of the M. elsdenii strains disclosed in International Patent Application Publication No. WO2021158927A1, incorporated by reference herein in its entirety), Clostridium butyricum, Bifidobacterium animalis ssp. animalis, Lactobacillus reuteri, Bacillus cereus, Lactobacillus salivarius ssp. Salivarius, Propionibacteria sp and combinations thereof.

A direct-fed microbial described herein comprising one or more bacterial strains may be of the same type (genus, species and strain) or may comprise a mixture of genera. species and/or strains. Alternatively, a DFM may be combined with one or more of the products or the microorganisms contained in those products disclosed in WO2012110778 and summarized as follows: Bacillus subtilis strain 2084 Accession No. NRRLB-50013, Bacillus subtilis strain LSSAO1 Accession No. NRRL B-50104, and Bacillus subtilis strain 15A-P4 ATCC Accession No. PTA-6507 (from Enviva Pro®. (formerly known as Avicorr®); Bacillus subtilis Strain C3102 (from Calsporin®); Bacillus subtilis Strain PB6 (from Clostat®); Bacillus pumilis (8G-134); Enterococcus NCIMB 10415 (SF68) (from Cylactin®); Bacillus subtilis Strain C3102 (from Gallipro® & GalliproMax®); Bacillus licheniformis (from Gallipro® Tect®); Enterococcus and Pediococcus (from Poultry Star®); Lactobacillus, Bifidobacterium and/or Enterococcus from Protexin®); Bacillus subtilis strain QST 713 (from Proflora®); Bacillus amyloliquefaciens CECT-5940 (from Ecobiol® & Ecobiol® Plus); Enterococcus faecium SF68 (from Fortiflora®); Bacillus subtilis and Bacillus licheniformis (from BioPlus2B®); Lactic acid bacteria 7 Enterococcus faecium (from Lactiferm®); Bacillus strain (from CSI®); Saccharomyces cerevisiae (from Yea-Sacc®); Enterococcus (from Biomin IMB52®); Pediococcus acidilactici, Enterococcus, Bifidobacterium animalis ssp. animalis, Lactobacillus reuteri, Lactobacillus salivarius ssp. salivarius (from Biomin C5®); Lactobacillus farciminis (from Biacton®); Enterococcus (from Oralin E1707®); Enterococcus (2 strains), Lactococcus lactis DSM 1103 (from Probios-pioneer PDFM®); Lactobacillus rhamnosus and Lactobacillus farciminis (from Sorbiflore®); Bacillus subtilis (from Animavit®); Enterococcus (from Bonvital®); Saccharomyces cerevisiae (from Levucell SB 20®); Saccharomyces cerevisiae (from Levucell SC 0 & SC10® ME); Pediococcus acidilactici (from Bactocell); Saccharomyces cerevisiae (from ActiSaf® (formerly BioSaf®)); Saccharomyces cerevisiae NCYC Sc47 (from Actisaf® SC47); Clostridium butyricum (from Miya-Gold®); Enterococcus (from Fecinor and Fecinor Plus®); Saccharomyces cerevisiae NCYC R-625 (from IntcSwinc®); Saccharomyces cerevisiae (from BioSprint®); Enterococcus and Lactobacillus rhamnosus (from Provita®); Bacillus subtilis and Aspergillus oryzae (from PepSoyGen-C®); Bacillus cereus (from Toyocerin®); Bacillus cereus var. toyoi NCIMB 40112/CNCM I-1012 (from TOYOCERIN®), or other DFMs such as Bacillus licheniformis and Bacillus subtilis (from BioPlus® YC) Bacillus subtilis (from GalliPro®), Propionibacterium acidipropionici (from Omni-Bos® P169), and Bacillus (from Omni-Bos® CB).

The DFM may be combined with Enviva® PRO which is commercially available from Danisco A/S. Enviva Pro® is a combination of Bacillus strain 2084 Accession No. NRRL B-50013, Bacillus strain LSSAO1 Accession No. NRRL B-50104 and Bacillus strain 15A-P4 ATCC Accession No. PTA-6507 (as taught in U.S. Pat. No. 7,754,469 B—incorporated herein by reference). Preferably, the DFM described herein comprises microorganisms which are generally recognized as safe (GRAS) and, preferably are GRAS-approved. A person of ordinary skill in the art will readily be aware of specific species and/or strains of microorganisms from within the genera described herein which are used in the food and/or agricultural industries and which are generally considered suitable for animal consumption.

In some embodiments, it is important that the DFM be heat tolerant, i.e. is thermotolerant. This is particularly the case when the feed is pelleted. Therefore, in another embodiment, the DFM may be a thermotolerant microorganism, such as a thermotolerant bacteria, including for example Bacillus spp.

In other aspects, it may be desirable that the DFM comprises a spore producing bacteria, such as Bacilli. e.g. Bacillus spp. Bacilli are able to form stable endospores when conditions for growth are unfavorable and are very resistant to heat, pH, moisture and disinfectants.

The DFM described herein may decrease or prevent intestinal establishment of pathogenic microorganism (such as Clostridium perfringens and/or E. coli and/or Salmonella spp and/or Campylobacter spp.). In other words, the DFM may be antipathogenic. The term “antipathogenic” as used herein means the DFM counters an effect (negative effect) of a pathogen.

As described above, the DFM may be any suitable DFM. For example, the following assay “DFM ASSAY” may be used to determine the suitability of a microorganism to be a DFM. The DFM assay as used herein is explained in more detail in US2009/0280090. For avoidance of doubt, the DFM selected as an inhibitory strain (or an antipathogenic DFM) in accordance with the “DFM ASSAY” taught herein is a suitable DFM for use in accordance with the present disclosure, i.e. in the feed additive composition according to the present disclosure. Tubes were seeded each with a representative pathogen (e.g., bacteria) from a representative cluster. Supernatant from a potential DFM, grown aerobically or anaerobically, is added to the seeded tubes (except for the control to which no supernatant is added) and incubated. After incubation, the optical density (OD) of the control and supernatant treated tubes was measured for each pathogen. Colonies of (potential DFM) strains that produced a lowered OD compared with the control (which did not contain any supernatant) can then be classified as an inhibitory strain (or an antipathogenic DFM). Thus, The DFM assay as used herein is explained in more detail in US2009/0280090. In some embodiments, a representative pathogen used in this DFM assay can be one (or more) of the following: Clostridium, such as Clostridium perfringens and/or Clostridium difficile, and/or E. coli and/or Salmonella spp and/or Campylobacter spp. In one preferred embodiment, the assay is conducted with one or more of Clostridium perfringens and/or Clostridium difficile and/or E. coli, preferably Clostridium perfringens and/or Clostridium difficile, more preferably Clostridium perfringens.

Antipathogenic DFMs include one or more of the following bacteria and are described in WO2013029013: Bacillus subtilis strain 3BP5 Accession No. NRRL B-50510, Bacillus amyloliquefaciens strain 918 ATCC Accession No. NRRL B-50508, and Bacillus amyloliquefaciens strain 1013 ATCC Accession No. NRRL B-50509.

DFMs may be prepared as culture(s) and carrier(s) (where used) and can be added to a ribbon or paddle mixer and mixed for about 15 minutes, although the timing can be increased or decreased. The components are blended such that a uniform mixture of the cultures and carriers result. The final product is preferably a dry, flowable powder. The DFM(s) comprising one or more bacterial strains can then be added to animal feed or a feed premix, added to an animal's water, or administered in other ways known in the art (preferably simultaneously with the enzymes described herein. Inclusion of the individual strains in the DFM mixture can be in proportions varying from 1% to 99% and, preferably, from 25% to 75% Suitable dosages of the DFM in animal feed may range from about 1×10³ CFU/g feed to about 1×10¹⁰ CFU/g feed, suitably between about 1×10⁴ CFU/g feed to about 1×10⁸ CFU/g feed, suitably between about 7.5×10⁴ CFU/g feed to about 1×10⁷ CFU/g feed. In another aspect, the DFM may be dosed in feedstuff at more than about 1×10³ CFU/g feed, suitably more than about 1×10⁴ CFU/g feed, suitably more than about 5×10⁴ CFU/g feed, or suitably more than about 1×10⁵ CFU/g feed.

The DFM may be dosed in a feed additive composition from about 1×10³ CFU/g composition to about 1×10¹³ CFU/g composition, such as 1×10⁵ CFU/g composition to about 1×10¹³ CFU/g composition, such as between about 1×10⁶ CFU/g composition to about 1×10¹² CFU/g composition, and such as between about 3.75×10⁷ CFU/g composition to about 1×10¹¹ CFU/g composition. In another aspect, the DFM may be dosed in a feed additive composition at more than about 1×10⁵ CFU/g composition, such as more than about 1×10⁶ CFU/g composition, and such as more than about 3.75×10⁷ CFU/g composition. In one embodiment, the DFM is dosed in the feed additive composition at more than about 2×10⁵ CFU/g composition, such as more than about 2×10⁶ CFU/g composition, suitably more than about 3.75×10⁷ CFU/g composition.

### III. Methods

### A. Methods for Increasing Starch Digestibility

Also provided herein is a method for increasing starch digestibility and glucose yield in the small intestine of a ruminant animal comprising adding at least one glucoamylase enzyme (such as any of the glucoamylase enzymes disclosed herein) and at least one acid stable alpha-amylase enzyme (AsAA; such as any of the AsAA enzymes disclosed herein) as a feed additive to feed for the ruminant.

In some embodiments, the method increases starch digestibility in a ruminant animal by any one of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% 35%, 36%, 37%, 38%, 39%, 40%, 41%, 42%, 43%, 44%, 45%, 46%, 47%, 48%, 49%, or 50% or more increased starch digestibility, compared to ruminant animals that are not fed a feed additive or feed comprising at least one glucoamylase enzyme (such as any of the glucoamylase enzymes disclosed herein) and at least one acid stable alpha-amylase enzyme (AsAA; such as any of the AsAA enzymes disclosed herein). In some embodiments, the at least one AsAA is at least about 60% (such as at least about 65%, 70%, 75%, 80%, 85%, 90%, 91%, 92%, 93%, 94%, 95%, 96%, 97%, 98%, or 100 identical, including values falling in between these percentages) to the amino acid sequence of SEQ ID NO: 3, SEQ ID NO: 4, or SEQ ID NO:6 or a variant or functional fragment thereof. In other embodiments, the at least one glucoamylase is at least about 60% (such as at least about 65%, 70%, 75%, 80%, 85%, 90%, 91%, 92%, 93%, 94%, 95%, 96%, 97%, 98%, or 100 identical, including values falling in between these percentages) to the amino acid sequence of SEQ ID NO:5 or SEQ ID NO: 7 or a variant or functional fragment thereof. Starch digestibility can be measured by any method known in the art, such as calculation of fecal starch content as described herein in Example 3.

In some embodiments, the method increases glucose yield in the small intestine of a ruminant animal by any one of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% 35%, 36%, 37%, 38%, 39%, 40%, 41%, 42%, 43%, 44%, 45%, 46%, 47%, 48%, 49%, or 50% or more increased glucose yield, compared to ruminant animals that are not fed a feed additive or feed comprising at least one glucoamylase enzyme (such as any of the glucoamylase enzymes disclosed herein) and at least one acid stable alpha-amylase enzyme (AsAA; such as any of the AsAA enzymes disclosed herein). In some embodiments, the at least one AsAA is at least about 60% (such as at least about 65%, 70%, 75%, 80%, 85%, 90%, 91%, 92%, 93%, 94%, 95%, 96%, 97%, 98%, or 100% identical, including values falling in between these percentages) to the amino acid sequence of SEQ ID NO: 3. SEQ ID NO:4, or SEQ ID NO:6 or a variant or functional fragment thereof. In other embodiments, the at least one glucoamylase is at least about 60% (such as at least about 65%, 70%, 75%, 80%, 85%, 90%, 91%, 92%, 93%, 94%, 95%, 96%, 97%, 98%, or 100% identical, including values falling in between these percentages) to the amino acid sequence of SEQ ID NO: 5 or SEQ ID NO:7 or a variant or functional fragment thereof. Glucose yield can be measured or calculated by any method known in the art.

In some embodiments, the method increases weight gain to feed ratio in a ruminant animal by any one of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% 35%, 36%, 37%, 38%, 39%, 40%, 41%, 42%, 43%, 44%, 45%, 46%, 47%, 48%, 49%, or 50% or more increased weight gain to feed ratio, compared to ruminant animals that are not fed a feed additive or feed comprising at least one glucoamylase enzyme (such as any of the glucoamylase enzymes disclosed herein) and at least one acid stable alpha-amylase enzyme (AsAA; such as any of the AsAA enzymes disclosed herein). In some embodiments, the at least one AsAA is at least about 60% (such as at least about 65%, 70%, 75%, 80%, 85%, 90%, 91%, 92%, 93%, 94%, 95%, 96%, 97%, 98%, or 100% identical, including values falling in between these percentages) to the amino acid sequence of SEQ ID NO: 3, SEQ ID NO:4, or SEQ ID NO:6 or a variant or functional fragment thereof. In other embodiments, the at least one glucoamylase is at least about 60% (such as at least about 65%, 70%, 75%, 80%, 85%, 90%, 91%, 92%, 93%, 94%, 95%, 96%, 97%, 98%, or 100% identical, including values falling in between these percentages) to the amino acid sequence of SEQ ID NO: 5 or SEQ ID NO:7 or a variant or functional fragment thereof.

### B. Methods for Increasing Milk Production

Also provided herein are methods for increasing milk production in a ruminant animal comprising adding at least one glucoamylase (EC 3.2.1.3) enzyme and at least one acid stable alpha-amylase (AsAA) enzyme as a feed additive to feed for the ruminant.

In some embodiments, the method increases milk production in a ruminant animal by any one of about 1%, 2%, 3%. 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34% 35%, 36%, 37%, 38%, 39%, 40%, 41%, 42%, 43%, 44%, 45%, 46%, 47%, 48%, 49%, or 50% or more increased milk production, compared to ruminant animals that are not fed a feed additive or feed comprising at least one glucoamylase enzyme (such as any of the glucoamylase enzymes disclosed herein) and at least one acid stable alpha-amylase enzyme (AsAA; such as any of the AsAA enzymes disclosed herein). In some embodiments, the at least one AsAA is at least about 60% (such as at least about 65%, 70%, 75%, 80%, 85%, 90%, 91%, 92%, 93%, 94%, 95%, 96%, 97%, 98%, or 100% identical, including values falling in between these percentages) to the amino acid sequence of SEQ ID NO: 3, SEQ ID NO:4, or SEQ ID NO: 6 or a variant or functional fragment thereof. In other embodiments, the at least one glucoamylase is at least about 60% (such as at least about 65%, 70%, 75%, 80%, 85%, 90%, 91%, 92%, 93%, 94%, 95%, 96%, 97%, 98%, or 100% identical, including values falling in between these percentages) to the amino acid sequence of SEQ ID NO:5 or SEQ ID NO:7 or a variant or functional fragment thereof.

### C. Methods for Producing AsAAs and Glucoamylases

### 1. Vectors

A DNA construct comprising a nucleic acid encoding an AsAA and/or glucoamylase polypeptide disclosed herein can be constructed such that it is suitable to be expressed in a host cell. Because of the known degeneracy in the genetic code, different polynucleotides that encode an identical amino acid sequence can be designed and made with routine skill. It is also known that, depending on the desired host cells, codon optimization may be required prior to attempting expression.

A polynucleotide encoding an AsAA and/or glucoamylase polypeptide of the present disclosure can be incorporated into a vector. Vectors can be transferred to a host cell using known transformation techniques, such as those disclosed below.

A suitable vector may be one that can be transformed into and/or replicated within a host cell. For example, a vector comprising a nucleic acid encoding an AsAA and/or glucoamylase polypeptide disclosed herein can be transformed and/or replicated in a bacterial host cell as a means of propagating and amplifying the vector. The vector may also be suitably transformed into an expression host, such that the encoding polynucleotide is expressed as a functional AsAA and/or glucoamylase enzyme.

A representative useful vector is pTrex3gM (see, Published U.S. patent application Ser. No. 20/130,323798) and pTTT (see, Published U.S. patent application No. 20110020899), which can be inserted into genome of host. The vectors pTrex3gM and pTTT can both be modified with routine skill such that they comprise and express a polynucleotide encoding an AsAA and/or glucoamylase polypeptide of the invention.

An expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the AsAA and/or glucoamylase to a host cell organelle such as a peroxisome, or to a particular host cell compartment. For expression under the direction of control sequences, the nucleic acid sequence of the AsAA and/or glucoamylase is operably linked to the control sequences in proper manner with respect to expression.

A polynucleotide encoding an AsAA and/or glucoamylase polypeptide disclosed herein can be operably linked to a promoter, which allows transcription in the host cell. The promoter may be any DNA sequence that shows transcriptional activity in the host cell of choice and may be derived from genes encoding proteins either homologous or heterologous to the host cell. Examples of promoters for directing the transcription of the DNA sequence encoding a glucoamylase, especially in a bacterial host, include the promoter of the lac operon of E. coli, the Streptomyces coelicolor agarase gene dagA or celA promoters, the promoters of the Bacillus licheniformis amylase gene (amyL), the promoters of the Bacillus stearothermophilus maltogenic amylase gene (amyM), the promoters of the Bacillus amyloliquefaciens amylase (amyQ), the promoters of the Bacillus subtilis xylA and xylB genes, and the like.

For transcription in a fungal host, examples of useful promoters include those derived from the gene encoding Aspergillus oryzae TAKA amylase, Rhizomucor miehei aspartic proteinase, Aspergillus niger neutral α-amylase, Aspergillus niger acid stable α-amylase, Aspergillus niger glucoamylase, Rhizomucor miehei lipase, Aspergillus oryzae alkaline protease, Aspergillus oryzae triose phosphate isomerase, Aspergillus nidulans acetamidase and the like. When a gene encoding a glucoamylase is expressed in a bacterial species such as an E. coli, a suitable promoter can be selected, for example, from a bacteriophage promoter including a T7 promoter and a phage lambda promoter. Along these lines, examples of suitable promoters for the expression in a yeast species include but are not limited to the Gal 1 and Gal 10 promoters of Saccharomyces cerevisiae and the Pichia pastoris AOX1 or AOX2 promoters. Expression in filamentous fungal host cells often involves cbh1, which is an endogenous, inducible promoter from T. reesei. See Liu et al. (2008) Acta Biochim. Biophys. Sin (Shanghai) 40(2): 158-65.

The coding sequence can be operably linked to a signal sequence. The DNA encoding the signal sequence may be a DNA sequence naturally associated with the AsAA and/or glucoamylase gene of interest to be expressed, or may be from a different genus or species as the AsAA and/or glucoamylase (i.e. the species from which the enzyme was derived). A signal sequence and a promoter sequence comprising a DNA construct or vector can be introduced into a fungal host cell and can be derived from the same source. For example, the signal sequence may be the Trichoderma reesei cbh1 signal sequence, which is operably linked to a cbh1 promoter.

An expression vector may also comprise a suitable transcription terminator and, in eukaryotes, polyadenylation sequences operably linked to the DNA sequence encoding a glucoamylase. Termination and polyadenylation sequences may suitably be derived from the same sources as the promoter.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., Published International PCT Application WO 91/17243.

### 2. Transformation and Culture of Host Cells

An isolated cell, either comprising a DNA construct or an expression vector, is advantageously used as a host cell in the recombinant production of an AsAA and/or glucoamylase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species.

Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A glucoamylase expressed by a fungal host cell can be glycosylated, i.e., will comprise a glycosyl moiety. The glycosylation pattern can be the same or different as present in the wild-type glucoamylase and/or AsAA. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. Any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding a glucoamylase is stably integrated into a host cell chromosome. Transformants are then selected and purified by known techniques.

### 3. Expression and Fermentation

A method of producing any of AsAAs and/or glucoamylases disclosed herein may comprise cultivating a host cell under conditions conducive to the production of the enzyme(s) and recovering the enzyme(s) from the cells and/or culture medium.

The medium used to cultivate the cells may be any conventional medium suitable for growing the host cell and obtaining expression of an AsAA and/or glucoamylase polypeptide. Suitable media and media components are available from commercial suppliers or may be prepared according to published recipes (e.g., as described in catalogues of the American Type Culture Collection).

Any of the fermentation methods well known in the art can suitably used to ferment the transformed or the derivative fungal strain as described above. In some embodiments, fungal cells are grown under batch or continuous fermentation conditions.

### 4. Methods for Enriching and Purification

Separation and concentration techniques are known in the art and conventional methods can be used to prepare a concentrated solution or broth comprising an AsAA and/or glucoamylase polypeptide of the invention.

After fermentation, a fermentation broth is obtained, the microbial cells and various suspended solids, including residual raw fermentation materials, are removed by conventional separation techniques in order to obtain a glucoamylase solution. Filtration, centrifugation, microfiltration, rotary vacuum drum filtration, ultrafiltration, centrifugation followed by ultrafiltration, extraction, or chromatography, or the like, are generally used.

It may at times be desirable to concentrate a solution or broth comprising an AsAA and/or glucoamylase polypeptide to optimize recovery. Use of un-concentrated solutions or broth would typically increase incubation time in order to collect the enriched or purified enzyme precipitate.

All references cited herein are herein incorporated by reference in their entirety for all purposes. In order to further illustrate the compositions and methods, and advantages thereof, the following specific examples are given with the understanding that they are illustrative rather than limiting.

### EXAMPLES

### Example 1: Preparation of A. niger Glucoamylase and Acidic Stable α-Amylase Sample

A sample containing the enzymes glucoamylase (AnGA, GenBank: XP_001390530.1 residues 25 to 640, SEQ ID NO: 5) and AniAmyl acidic stable α-amylase (AsAA, GenBank: ADX42122.1 residues 1 to 484, SEQ ID NO:6) was obtained from Aspergillus niger strain fermentation and assayed as described here. The glucoamylase activity was measured using the Megazyme amyloglucosidase assay kit (R-AMGR3) containing p-nitrophenyl β-maltoside and a thermostable β-glucosidase. A sample of glucoamylase product Diazyme® TGA (IFF) was used as reference standard. The reaction included 5-10 μl of the diluted enzyme, 50 μl 0.1M MES buffer (pH6.5) and 50 μl the substrate mixture per well of a 96-well plate. The absorbance at 400 nm was measured in a time course using a microplate reader (Bio-Tek). The glucoamylase activity in the sample was 248.1 GA units/g. The α-amylase activity was determined using the Amylazyme tablet reagent (Megazyme) following method described by manufacturer. The reaction buffer consisted of 0.1M HAC pH4.0 with 5 mM CaCl₂ and 0.01% (v/v) Tween 80. A sample of α-amylase product Axtra® XAP (IFF) was used as standard with known α-amylase units. One α-amylase unit is the amount of enzyme required to release 0.20 μmol of glucosidic linkages (expressed as p-nitrophenol equivalents) from a maltoheptaoside substrate per minute at pH 8.0 and 40° C. (in the presence of excess α-glucosidase). The α-amylase activity measured in the sample was 220.7 units/g.

### Example 2: PH Effect on Hydrolysis of Corn Starch Using the A. Niger Glucoamylase and Acidic Stable α-Amylase Sample

The degree of corn starch degradation over a pH range of 3.0 to 6.5 was evaluated. The reaction mixture contained 200 mg corn starch (Sigma S-4126), 30 μl of a sample containing 6.6 TGA units of A. niger glucoamylase (AnGA, SEQ ID NO: 5) and 5.4 α-amylase units acidic stable α-amylase (AsAA, SEQ ID NO: 6) mixed in 10 ml of buffer: 0.1 M sodium acetate (pH 3.5-5.5) or 0.1 M Mes-NaOH (pH 6.0-6.5) containing 5 mM CaCl₂). The reaction was carried out at 40° C. with shaking at 1240 rpm for 40 min. The corn hydrolysis was measured as glucose released, analyzed by using glucose oxidase peroxidase kit from Megazyme (Cat. No. K-GLUC). Sample aliquots of 0.2 ml were transferred to a tube having 0.2 ml 2% Tris base and mixed to stop the enzyme reactions. 10 μl of each mixture was transferred to 96 well plate and mixed with 0.2 ml the Megazyme reagent, mixed at 40° C., for 20 min before the measurement of OD at 510 nm. Table 1 shows the release of glucose from corn starch catalyzed by the AnGA and AsAA sample as a function of pH.

TABLE 1
Corn starch hydrolysis by A niger GA and AsAA sample.

| pH | 3.0 | 3.5 | 4.0 | 4.5 | 5.0 | 5.5 | 6.0 | 6.5 |
|---|---|---|---|---|---|---|---|---|
| Glucose | 17.6 | 19.2 | 17.6 | 14.8 | 11.8 | 8.7 | 6.4 | 4.6 |
| released | | | | | | | | |
| (mg) | | | | | | | | |

### Example 3: Evaluation of A niger GA and AsAA on Beef Cattle Growth

A sample derived from A. niger culture containing glucoamylase AnGA (SEQ ID NO: 5) with activity at 248.1 TGA unit/g, and alpha-amylase AsAA (SEQ ID NO:6) with activity at 220.7 Units/g was used in a beef cattle performance trial at dose of 54.0 mg AnGA and 2.5 mg AsAA per kilo feed. In this trial, steers were fed with and without enzymes for 136 days. Specifically, the effect of delivering a blend of AnGA and AsAA on cattle growth performance was tested using 70 Angus×Simmental steers fed for 136 day at the Beef and Sheep Field Research Laboratory in Urbana, IL (USA). The experiment was conducted as a randomized block design. Steers were blocked by BW, stratified by sire, and assigned to 10 pens with 7 steers in each pen. Pens were assigned randomly to one of two treatments. Treatments included a finishing diet with no enzyme addition (negative control; CON), and the finishing diet with exogenous enzymes.

The finishing diets were offered ad libitum in GrowSafe (GrowSafe Systems Ltd, Calgary, AB, Canada) bunks for the duration of the study. Feed ingredients samples were collected biweekly and frozen at −20° C., and composited at the end of the experiment. Composite samples were lyophilized and ground in a Wiley Mill. Total tract starch digestibility was calculated from fecal starch content using an equation reported by Owens et al. (Owens et al., 2016. Prof. Anim. Sci. 32:531-549), where Digestibility %=0.0102×FS²−0.3621×fecal starch+99.701. The results of this treatment are shown in Table 2.

TABLE 2
Effect of exogenous AnGA and AsAA on growth
of cattle supplemented for 136 days.

| | | GA and | | |
|---|---|---|---|---|
| | Untreated | AsAA | | Treatment |
| Item | Control | treatment | SEM | p-value |
| Body weight, kg, Start | 339 | 336 | 4.1 | 0.847 |
| Body weight, kg, End | 595 | 601 | 7.3 | 0.689 |
| Average Daily Gain, kg/d | 1.860 | 1.923 | 0.055 | 0.447 |
| Dry Matter Intake, kg/d | 9.5 | 9.1 | 0.24 | 0.837 |
| Gain:Feed | 0.198 | 0.217 | 0.0051 | 0.003 |
| Dressing, % | 64.0 | 63.9 | 0.30 | 0.842 |
| Total tract starch | 80.3 | 85.1 | 5.83 | 0.312 |
| digestibility, % | | | | |
| 12th Rib fat, cm | 1.27 | 1.42 | 0.037 | 0.032 |

In Table 2, it can be seen that treatment with exogenously added AnGA and AsAA enzymes improved the Weight Gain to Feed ratio from 0.198 (Control) to 0.217 (plus enzymes), an improvement of 12.0%. Additionally, we observed a rib fat thickness improvement from 1.27 to 1.42 cm in 12th rib, and a total tract starch digestibility improvement for 80.3 to 85.1%.

### Example 4: Acidomyces richmondensis Alpha Amylase (AriAmy1) Cloning and Expression

A search for alpha amylase enzymes was performed by scanning the proteins of Acidomyces richmondensis BFW using dbCAN [Yanbin Yin et al., Nucleic Acids Res. 2012 July; 40]. A number of putative alpha amylases were identified and one of them, AriAmy1 (scaffold_85:10600-13013, protein ID: 3015 Hypertext Transfer Protocol Secure//mycocosm.jgi.doe.gov/cgi-bin/dispGeneModel?db=Aciri1_iso&id=3015) was further analyzed. The gene encoding AriAmy1 was assigned as SEQ ID NO: 1.

The N-terminal signal peptide was predicted by SignalP software version 4.0 (Nordahl Petersen et al. (2011) Nature Methods, 8:785-786). The gene encoding AriAmy1 was codon modified for expression in Trichoderma reesei. The codon optimized AriAmy1 gene sequence, the full length protein sequence and the predicted mature protein sequence were assigned as SEQ ID NO: 2, 3, 4 respectively.

The gene encoding AriAmy1 (SEQ ID NO: 2) was synthesized by Generay (Generay Biotech Co., Ltd, Shanghai, China) and inserted into the pGX256 expression vector, a derivative vector from pTTT (see, Published U.S. patent application No. 20110020899). The plasmids were transformed into a suitable Trichoderma reesei strain using protoplast transformation (Te'o et al., J. Microbiol. Methods 51:393-99, 2002). The transformants were selected and fermented by the methods described in WO 2016/138315, incorporated by reference herein. Supernatants from these cultures were used to confirm the protein expression by SDS-PAGE analysis and select strain for further evaluations. Fungal cell cultures were grown in a defined medium (Lv D et al., Plasmid. 67(1):67-71, 2012) and clarified culture broth was collected after 96 hours by centrifugation. The alpha amylase AriAmy1 protein was purified by methods known in the art. The column chromatography fractions containing the target protein were pooled, concentrated and equilibrated to 20 mM sodium acetate pH 5.0, containing 150 mM sodium chloride using an Amicon Ultra-15 device with 10 K filter (Millipore). The purified AriAmy1 samples were approximately 99% pure (by SDS-PAGE analysis) and were stored in 40% glycerol at −80° C. until use.

### Example 5: Low pH and Pepsin Stability of AriAmy1

The low pH stability of fungal alpha-amylases AriAmy1 (SEQ ID NO:4) and A. niger AsAA (AniAmyl, SEQ ID NO:6) were evaluated by pre-incubating the enzyme working solutions at pH 2, 40° C. The enzyme working solutions (10 ppm) were prepared in 20 mM of glycine-HCl buffer (pH=2) or in 5 mg/mL pepsin in 0.1 M HCl (pH=2). Then they were pre-incubated at 40° C., for 15 min, 30 min, 60 min, 120 min, respectively. The enzyme working solution prepared in water was used as a control as 0-min point. The alpha-amylases residual activity was measured by incubating 5 μL of the above enzyme working solution with 45 μL of Ceralpha kit (R-CAAR4, Megazyme) at pH 5.0, 50° C., for 10 min with shaking (650 rpm) in an iEMS incubator (ThermoFisher). The reaction was quenched by adding 50 μL of 1 M sodium carbonate. The microtiter plate containing the reaction mixture was measured at 405 nm. Enzyme activity with 0-min preincubation (enzyme working solution prepared in water) was used as 100% of alpha-amylase activity. As shown in Table 3, AriAmy1 maintains 62% of its activity after pre-incubation in glycine-HCl buffer (pH=2) at 40° C., for 120 min and 48% of its activity after pre-incubation with 5 mg/mL pepsin in 0.1 M HCl (pH=2) at 40° C., for 120 min. The AsAA [see notes above] amylase retained 46% of its activity after pre-incubation in glycine-HCl buffer (pH=2) at 40° C., for 120 min and 12% of its activity after pre-incubation with 5 mg/mL pepsin in 0.1 M HCl (pH=2) at 40° C., for 120 min.

TABLE 3
Low pH and pepsin treatment stability of alpha-amylases.

| Preincubation | 20 mM glycine-HCl | | 5 mg/mL pepsin | |
|---|---|---|---|---|
| time | buffer (pH 2) | | (pH 2) | |
| (min) | AniAmy1 | AriAmy1 | AniAmy1 | AriAmy1 |
| 120 | 46 | 62 | 12 | 48 |
| 60 | 55 | 70 | 27 | 49 |
| 30 | 57 | 63 | 29 | 53 |
| 15 | 63 | 66 | 39 | 49 |
| 0 | 100 | 100 | 100 | 100 |

### Example 6: Hydrolysis of Corn Starch Using Acidic Stable α-Amylase AriAmy1 and Acid-Stable Glucoamylase WcoGA1

The reaction mixture contained 30 mg corn starch (Sigma S-4126) in 1 mL 0.1M sodium acetate (pH4.5) containing 0.01% Tween-80, and 70 μg glucoamylase WcoGA1 (SEQ ID NO: 7), 30 ug α-amylase AriAmy1 (SEQ ID NO: 4), or a mixture of both enzymes, and was incubated at 40° C. Samples were evaluated as described on Example 2 above. Table 4 shows the release of glucose from corn starch as a function of time. Glucose release with α-amylase alone was not detectable. A clear synergy in glucose release was seen when both enzymes were added in terms of glucose released (mg).

TABLE 4
Corn starch digestion using WcoGA1 and AriAmy1 blend.

| Enzyme(s)/Incubation time (h) | 0.5 | 1.0 | 1.5 | 3.0 |
|---|---|---|---|---|
| Glucoamylase WcoGA1 | 0.267 | 0.410 | 0.519 | 0.972 |
| α-Amylase AriAmy1 | 0 | 0 | 0 | 0 |
| Glucoamylase WcoGA1 + | 0.565 | 1.209 | 1.497 | 2.823 |
| α-amylase AriAmy1 | | | | |

### Example 7: Identification of Additional Acid Stable Fungal Alpha-Amylases

A search for alpha amylase enzymes was performed by scanning the proteins of various fugal genomes using dbCAN (Yanbin Yin et al., Nucleic Acids Res. 2012 July; 40). A number of putative alpha amylases were identified and listed below on Table 5 and Table 6. The percent (%) sequence identity was determined by protein sequence alignment using the CLUSTAL W program over the predicted mature sequences. Homologs of AriAmy1 (SEQ ID NO:4) are reported in Table 5. Homologs of AniAmyl (SEQ ID NO: 6) are reported in Table 6. The AriAmy1 and homologs appear to have a C-terminal CBM20 domain.

TABLE 5
Alpha amylases with sequence relationship to AriAmy1 (SEQ ID NO:4)

| | | % | Predicted | |
|---|---|---|---|---|
| Identifier/ | | | mature | |
| accession | Source | identity to | sequence | |
| # | organism | AriAmy1 | (SID No) | Predicted mature sequence |
| AriAmy1 | Acidomyces | 100 | 4 | LSPAGWREQSIYQVVTDRFALTSGGSSPSC |
| | richmondensis | | | TSDFQLSIFCNGTFRGIIDKLDYIQGMGFTA |
| | BFW | | | MWISPIVENIKGGTPDGYTEDGSAYHGYW |
| | | | | AQDIYAINPHFGTPEDLVALAEALHNRGM |
| | | | | YLMVDIVVNHMGYYCGKAEDGHCGPQGT |
| | | | | IDYSIYNPENSEKYFHPFCEINYNNATSILVC |
| | | | | WEGDEIVPLPDLRTENTTVQSMFDTWITEL |
| | | | | VKNYTIDGLRIDSLQQSGSFFFPSFYKAAGE |
| | | | | LYMVGEVFQEYPAKVCPYQQDGMPGVLN |
| | | | | YPMFYYITDAFQDSTGSMSGLAEGISYMQ |
| | | | | GNCSDTTLLGSFLENQDNPRFPSYTEDITRA |
| | | | | KNAIAFTMLQDGIPIIYYGQEQHLNGSGVP |
| | | | | QNREALWSSGGYNTASMLYNMIKEVNAIR |
| | | | | TQAIHVSQSFVTEKITTPYYDNHTIVTRKGE |
| | | | | TGSQIIGVYSNLGENGAYYNLSLKSAVTGF |
| | | | | GNEEKIMEILTCTLLTTSSDGEIIVPMGEGQ |
| | | | | PKILFPFARLSGSSICKSYKTISPVGIDCEPA |
| | | | | TITFDILVNTTYGESVVVAGDIPELGDWNV |
| | | | | PDGLYLNASKYTTENPLWTGTATGIDPQEV |
| | | | | FQWKPVVILTNGSYVYAPGDNIETVAPGPG |
| | | | | CNPTATIKYVFSGQ |
| fADZ99364.1 | Bispora sp. | 99.8 | 15 | LSPAGWREQSIYQVVTDRFALTSGGSSPSC |
| | MEY-1 | | | TSDFQLSIFCNGTFRGIIDKLDYIQGMGFTA |
| | | | | MWISPIVENIKGGTPDGYTEDGSAYHGYW |
| | | | | AQDIYAINPHFGTPEDLVALAEALHNRGM |
| | | | | YLMVDIVVNHMGYYCGKAEDGHCGPQGT |
| | | | | IDYSIYNPENSEKYFHPFCEINYNNATSILVC |
| | | | | WEGDEIVPLPDLRTENTTVQSMFDTWITEL |
| | | | | VKNYTIDGLRIDSLQQSGSFFFPSFYKAAGE |
| | | | | LYMVGEVFQEYPAKVCPYQQDGMPGVLN |
| | | | | YPMFYYITDAFQDSTGSMSGLAEGISYMQ |
| | | | | GNCSDTTLLGSFLENQDNPRFPSYTEDITRA |
| | | | | KNAIAFTMLQDGIPIIYYGQEQHLNGSGVP |
| | | | | QNREALWSSGGYNTASMLYNMIKEVNAIR |
| | | | | TQAIHVSQSFVTEKITTPYYDNHTIVTRKGE |
| | | | | TGSQIIGVYSNLGENGAYYNLSLKSAVTGF |
| | | | | GNEEKIMEILTCTLLTTSSDGEIIVPMGEGQ |
| | | | | PKILFPFARLSGSSICKSYKTISPVGIDCEPA |
| | | | | TITFDILVNTTYGESVVVAGDIPELGDWNV |
| | | | | PDGLYLNASKYTTENPLWTGTATGIDPQEV |
| | | | | FQWKPVVILTNGSYVYAPGDNIETVAPGPG |
| | | | | CNPTATIEYVFSGQ |
| Symko1_ | Symbiotaphrina | 76.7 | 16 | LSPAGWREQSIYQIVTDRFALTSGGSTPACT |
| 787523 | kochii | | | TDTQLGMFCNGTFQGIINKLDYIQNMGFTA |
| | | | | IWISPIVKNIDGGSPDGYTVDGSAYHGYWA |
| | | | | QDIYQINPHFGTPQDLKQLSQSLHQRGMYL |
| | | | | MVDIVVNHNGYYCGLAEDGNCGPQGTIDY |
| | | | | SIYNPENSEEYFHPFCEIDYSNATSILDCWE |
| | | | | GDEIVPLPDLRTENTTVQAMFDSWIKELVN |
| | | | | NYTIDGLRIDSLQQSGSFFFPSFYSASGGLY |
| | | | | MVGEVFQEYPAQVCPYQQAGMPGVLNYP |
| | | | | MFYYITDAFEDSAGSMSALANGISYMQGN |
| | | | | CSDTTLLGSFLENQDNPRFPTTTGDIVRAM |
| | | | | NAITFTLLQDGIPITYYGQEQHLNGSGVPEN |
| | | | | RQALWSSGGYDTTSQLYEMISKVNAIRTH |
| | | | | AIEKASSFVTDKIQTPYYDNHTIVTRKGDP |
| | | | | GAQIVGVYSNLGENGDYYDLTLTKEETGF |
| | | | | GPIEPVMEILTCTLLTTSFSGDLTVPMGGGQ |
| | | | | PRILFPYLGIIGSQICPGFTLKPPTGPNCSPAE |
| | | | | ITFEILANTTWGESVVVTGDIPSLGNWDVA |
| | | | | NGVHLNASEYTAANPLWVGTATGIDPQEV |
| | | | | FQWKPVVIQTDGSYDWYDGDNIQDVSPGP |
| | | | | GCNPTGTLQVTFY |
| Lorju1_ | Loramyces | 69.9 | 17 | LSGADWRQQSIYQVVIDRFSLTNGDLTPKC |
| 512576 | juncicola | | | TTDFELSNYCNGTFQGIISQLNYIQGMGFTS |
| | 46458 | | | IWISPIVKNIEGGSPNGYTKDGSPYHGYWA |
| | | | | QDIYAINPHFGTEQDLKDLASELHDRGMFL |
| | | | | MVDIVVNHMGYYCGAAEEGSCGPEGSIDY |
| | | | | SVFNPFDDESYFHSFCLIDYNNATSILDCWE |
| | | | | GSQNVPLPDLRIEDVSVQKILDSWITDLVK |
| | | | | NYSIDGLRIDSLQQSGSFFFPSFSNATGEIY |
| | | | | MVGEVFQEYPAQVCPYQQPGMPGVMNYP |
| | | | | MYYYINDAFKSSSGNMKALSEGIAYMQGN |
| | | | | CTDTTLLGSFLENQDNARFPSYTGDIVRAA |
| | | | | NAITFTLLQDGIPITYYGQEQHLNGTGTPYN |
| | | | | REALWSTGGYNTSSTLYKLISQVNAIRTKA |
| | | | | SELSSNFLTSKIKTPYTDDHTIVTCKGEAGN |
| | | | | QIIGVYSNLGENGAHYNLTLSKTVTGENDS |
| | | | | QVIVEVLSCTLSTTSPSGDLTLPMGAGEPK |
| | | | | VLFPYSVLQNSSICPNPSSPPTAPGQNCSSA |
| | | | | SITFNILVNTTYGESVVVAGNIPALGNWNV |
| | | | | PSGLSLSADKYTAENPLWSGTATGIDPQAA |
| | | | | FQWKPVVVKTDGSYVYLPENNLMDVAPG |
| | | | | PGCIPSGTIGYKWLSS |
| XP_ | Kockovaella | 65.5 | 18 | LSPEQWRAQSIYQVVTDRFALDGGALSPTC |
| 021873327.1 | imperatae | | | TGDFELSIFCNGSFYGLIDKLQYIKDMGFTA |
| | | | | LWVSPVVENIEGGTPDGYTKDGSPYHGYW |
| | | | | AKDIYSINDHFGGSDGLKALSQALHNNDM |
| | | | | YLMVDVVVNHMGYYCGAALDGHCGPQG |
| | | | | TVNYSIYNPENSEKYFHPFCEIDYNNATSIL |
| | | | | DCWEGSENVPLPDLRTEDTVVQQMFDSWI |
| | | | | SGLVSDYSIDGLRIDSLQQSGSFFFPSFFNAT |
| | | | | GGMYMVGEVFQEYPKRVCSYQQEGMPGV |
| | | | | LNYPMFYYVTDAFSTSDGSMSGLANGISY |
| | | | | MRGNCSDVTLLGSFLENQDNPRFPSLTEDI |
| | | | | TRSMNAITFVMLQDGIPIVYYGQEQHLNGG |
| | | | | EVPYNREALWSTGGYNQSAPLYEMITSINK |
| | | | | ARTAAIQQDASFVTDEMQIPYYDDHVIVAR |
| | | | | KGRSGDQIVSIYDNRGSSGASSGLTLSQTD |
| | | | | TGFSSAEQVMDILSCEQYTTASDGSLFMPL |
| | | | | GGGLPKALVTQSVLESSGLCTTYTADSVA |
| | | | | GPTSVGSSTTCANATVVFDIYVTTSYGQSV |
| | | | | VIAGDISALGDWTPANGLKLSANGYTADSP |
| | | | | LWTGSAQVDPGTVFQWKPVIIETNESEDW |
| | | | | YSGDNLQNASQAGCHPQSTTILLDWTS |
| BCG553891 | Saitozyma | 63.8 | 19 | AGLTPAEWRSQSIYQVVTDRFALDNGGNS |
| | flava | | | PSCSGQSELNLYCNGTFAGIIDKLDYIQNM |
| | | | | GFTAIWISPVVKNIDGGSPNGYTPDGSAYH |
| | | | | GYWAQDIYEINPHFGGASGLTDLSNALHSR |
| | | | | GMYLMVDVVVNHMAYYCGTDGGCGPGN |
| | | | | SVNYGSFTPFNSESYFHPFCEIDYNNRTSIL |
| | | | | DCWEGDEIVPLVDLRTEDSDVQSIFNSWIS |
| | | | | NLIQTYNIDGLRIDSLQQSGSFFFPGFNQAA |
| | | | | GGMYMVGEVFNGSPSYVCPYQQAGMPGV |
| | | | | LNYPMFFYITNAFQTSSGSMSQLAQGISAM |
| | | | | QSDCSDTTLLGSFLENQDNPRFPSQTSDLT |
| | | | | RAQNAIAFTMLQDGIPITYYGQEQHLSGSG |
| | | | | VPLNREALWTSGGYDTSSPLYEMITTVNQL |
| | | | | RTLAIKQNGGFVTYKIQVPYTDSNHIVTRK |
| | | | | GNSGYQIVGVYTNVGSAGASSTLSISSSETG |
| | | | | FQASEPVMDVLSCTLYHTGTDGSLSFTMT |
| | | | | GGLPRVFYNATALAESSLCTTYTTASPPPG |
| | | | | GCSAGTVVFDVYVQTQYGQSVVIAGNIPQ |
| | | | | LGNWSPANGLNLNANQYTASSPKWTGTIT |
| | | | | GVAPGTTFQWKPIVVTNGNDNWYPGNNQ |
| | | | | QATTGSACSS |
| BAA12010.1 | Cryptococcus | 62.7 | 20 | LSPAEWRSQSIYQVVTDRFALDNGGNSPSC |
| | sp. S-2 | | | SGQSELNLYCNGTFAGIIDKLDYIQNMGFT |
| | | | | AIWISPVVKNIDGGSPNGYTPDGSAYHGY |
| | | | | WAQDIYEINPHFGGASGLTDLSNALHSRG |
| | | | | MYLMVDVVVNHMAYYCGTDGGCGPGNS |
| | | | | VNYGSFTPFNSESYFHPFCEIDYNNRTSILD |
| | | | | CWEGDEIVPLVDLRTEDSDVQSIFNSWISN |
| | | | | LIQTYNIDGLRIDSLQQSGSFFFPGFNQAAG |
| | | | | GMYMVGEVFNGSPSYVCPYQQAGMPGVL |
| | | | | NYPMFFYITNAFQTSSGSMSQLAQGISAMQ |
| | | | | SDCSDTTLLGSFLENQDNPRFPSQTSDLTR |
| | | | | AQNAIAFTMLQDGIPITYYGQEQHLSGSGV |
| | | | | PLNREALWTSGGYDTSSPLYEMITTVNQLR |
| | | | | TLAIKQNGGFVTYKIQVPYTDSNHIVTRKG |
| | | | | NSGYQIVGVYTNVGSAGSSSTLSISSSETGF |
| | | | | QASEPVMDVLSCTLYHTGTDGSLSFTMTG |
| | | | | GLPRVFYNATALAESSLCTTYTTASPPPGG |
| | | | | CSAGTVVFDVYVQTQYGQSVVIAGNIPQL |
| | | | | GNWSPANGLNLNANQYTASSPKWTGTITG |
| | | | | VAPGTTFQWKPIVVTNGNDNWYPGNNQQ |
| | | | | ATTGSACSSPAADIEFTWSS |
| ABS76467.1 | Saitozyma | 62.5 | 21 | LSPAEWRSQSIYQVVTDRFALDNGGNSPSC |
| | flava | | | SGQSELNLYCNGTFAGIIDKLDYIQNMGFT |
| | | | | AIWISPVVKNIDGGSPNGYTPDGSAYHGY |
| | | | | WAQDIYEINPHFGGASGLTDLSNALHNRG |
| | | | | MYLMVDVVVNHMAYYCGTNGGCGPGNS |
| | | | | VNYGSFTPFNSESYFHPFCEIDYNNRTSILD |
| | | | | CWEGDEIVPLVDLRTEDSDVQSIFNSWISN |
| | | | | LIQTYNIDGLRIDSLQQSGSFFFPGFNQAAG |
| | | | | GMYMVGEVFNGNPSYVCPYQQAGMPGVL |
| | | | | NYPMFFYITNAFQTSSGSMSQLAQGISAMQ |
| | | | | SDCSDTTLLGSFLENQDNPRFPSQTNDLTR |
| | | | | AQNAIAFTMLQDGIPITYYGQEQHLSGSGV |
| | | | | PLNREALWTSGGYDSSSPLYKMITTVNQLR |
| | | | | TLAIKQNGGFVTYKIQVPYTDSNHIVTRKG |
| | | | | NSGYQIVGVYTNVGSAGASSTLSLSSSETG |
| | | | | FQASEPVMDVLSCTLYHTGSDGSLSFTMT |
| | | | | GGLPRVFYNATALAESSLCTTYTTATPPPG |
| | | | | GCSAGTVVFDVYVQTQYGQSVVIAGNIPQ |
| | | | | LGNWSPANGLNLNANQYTASSPKWTGTIT |
| | | | | GVAPGTTFQWKPIVVTNGNDNWYTGSNQ |
| | | | | QATTGSACSSPATDIEFTWSS |

TABLE 6
Alpha amylases with sequence relationship to AsAA AniAmy1 (SEQ ID No 6)

| | | | Predicted | |
|---|---|---|---|---|
| | | % | mature | |
| Identifier/ | | identity to | sequence | |
| accession # | Source organism | AniAmy1 | (SID No) | Predicted mature sequences |
| AniAmy 1 | Aspergillus niger | 100 | 6 | LSAAEWRTQSIYFLLTDRFGRTD |
| (AsAA) | | | | NSTTATCDTGDQIYCGGSWQGII |
| | | | | NHLDYIQGMGFTAIWISPITEQLP |
| | | | | QDTADGEAYHGYWQQKIYDVN |
| | | | | SNFGTADDLKSLSDALHARGMY |
| | | | | LMVDVVPNHMGYAGNGNDVDY |
| | | | | SVFDPFDSSSYFHPYCLITDWDN |
| | | | | LTMVQDCWEGDTIVSLPDLNTTE |
| | | | | TAVRTIWYDWVADLVSNYSVDG |
| | | | | LRIDSVLEVEPDFFPGYQEAAGV |
| | | | | YCVGEVDNGNPALDCPYQKVLD |
| | | | | GVLNYPIYWQLLYAFESSSGSISN |
| | | | | LYNMIKSVASDCSDPTLLGNFIE |
| | | | | NHDNPRFASYTSDYSQAKNVLS |
| | | | | YIFLSDGIPIVYAGEEQHYSGGKV |
| | | | | PYNREATWLSGYDTSAELYTWI |
| | | | | ATTNAIRKLAISADSAYITYAND |
| | | | | AFYTDSNTIAMRKGTSGSQVITV |
| | | | | LSNKGSSGSSYTLTLSGSGYTSGT |
| | | | | KLIEAYTCTSVTVDSSGDIPVPM |
| | | | | ASGLPRVLLPASVVDSSSLCGGS |
| | | | | GRLYVE |
| XP_ | Aspergillus | 99.6 | 22 | MRLSTSSLFLSVSLLGKLALGLS |
| 026629502.1 | welwitschiae | | | AAEWRTQSIYFLLTDRFGRTDNS |
| | | | | TTATCDTGDQIYCGGSWQGIINH |
| | | | | LDYIQGMGFTAIWISPITEQLPQD |
| | | | | TADGEAYHGYWQQKIYDVNSNF |
| | | | | GTADDLKSLSDALHARGMYLM |
| | | | | VDVVPNHMGYAGNGNDVDYSV |
| | | | | FDPFDSSSYFHPYCLITDWDNLT |
| | | | | MVQDCWEGDTIVSLPDLNTTET |
| | | | | AVRTIWYDWVADLVSNYSVDGL |
| | | | | RIDSVLEVEPDFFPGYQEAAGVY |
| | | | | CVGEVDNGNPALDCPYQKVLDG |
| | | | | VLNYPIYWQLLYAFESSSGSISDL |
| | | | | YNMIKSVASDCSDPTLLGNFIEN |
| | | | | HDNPRFASYTSDYSQAKNVLSYI |
| | | | | FLSDGIPIVYAGEEQHYSGGKVP |
| | | | | YNREATWLSGYDTSAELYTWIA |
| | | | | TTNAIRKLAISADSAYITYANDAF |
| | | | | YTDSNTIAMRKGTSGSQVITVLS |
| | | | | NKGSSGSSYTLTLSGSGYTSGTK |
| | | | | LIEAYTCTSVTVDSSGDIPVPMAS |
| | | | | GLPRVLLPASVVDSSSLCGGSGS |
| | | | | NSSTTTTTTATSSSTATSKSASTS |
| | | | | STSTACTATSTSLAITFEELVTTT |
| | | | | YGEEIYLSGSISQLGDWDTSDAV |
| | | | | KMSADDYTSSNPEWSVTVTLPV |
| | | | | GTTFEYKFIKVESDGSVTWESDP |
| | | | | NREYTVPECGSGETVVDTWR |
| OJJ77724.1 | Aspergillus | 96.4 | 23 | MRLSTSILALSVSLLGKLALGLSA |
| | brasiliensis CBS | | | AEWRTQSIYFLLTDRFGRTDNST |
| | 101740 | | | TATCNTGDQIYCGGSWQGIINHL |
| | | | | DYIQGMGFTAIWISPITEQLPQDT |
| | | | | SDGEAYHGYWQQKIYDVNSNFG |
| | | | | TADDLKSLSDALHARGMYLMID |
| | | | | VVPNHMGYAGSGNDVDYSVFDP |
| | | | | FDSSSYFHPYCLITDWDNLTMVQ |
| | | | | DCWEGDTIVSLPDLNTTETVVRT |
| | | | | IWYDWVADLVSNYSVDGLRIDS |
| | | | | VLEVEPDFFPGYQEAAGVYCVG |
| | | | | EVDNGNPALDCPYQDYLDGVLN |
| | | | | YPIYWQLLYAFESSSGSISDLYN |
| | | | | MIKSVASDCSDPTLLGNFIENHD |
| | | | | NPRFAYYTSDYSQAKNVLSYIFL |
| | | | | SDGIPIVYAGEEQHYSGGDVPYN |
| | | | | REATWLSGYDTSAELYTWIATT |
| | | | | NAIRKLAIAADSSYITYANDPIYT |
| | | | | DSNTIAMRKGTSGSQVITVLSNK |
| | | | | GSSGSSYTLTLSGSGYTSGTKLIE |
| | | | | AYTCTSVTVDSNGDIPVPMASGL |
| | | | | PRVLLPASVVDDSSLCGGSGSST |
| | | | | STTTSTATATTTSKTSTTSSSSSSS |
| | | | | SCTASATAIPITFEELVTTTYGEEI |
| | | | | YLSGSISQLGDWDTSDAVKLSAD |
| | | | | DYTSSNPEWSVTVTLPVGTTFEY |
| | | | | KFIKVESGGSVTWESDPNREYTV |
| | | | | PECGSGETVVDTWR |
| PYI11254.1 | Aspergillus | 92.4 | 24 | MKLSSSSLLLSAPLFGKLALGLS |
| | sclero- | | | AAEWRSQSIYFLLTDRFGRTDNS |
| | tiicarbonarius | | | TTATCDTGEQIYCGGSWQGVINH |
| | CBS 121057 | | | LDYIQGMGFTAIWISPVTEQLPQ |
| | | | | DTADGEAYHGYWQQEIYTVNSN |
| | | | | FGTADDLKALSDALHARGMYL |
| | | | | MVDVVPNHMGYAGDGDDVDYS |
| | | | | VFDPFDSSSYFHPYCLITDWSNLT |
| | | | | MVRDCWEGDTIVSLPDLNTTET |
| | | | | VVRTIWYDWVADLVANYSVDG |
| | | | | LRIDSALEVEPDFFPGYQEAAGV |
| | | | | YCVGEVDNGDPTLDCPYQEYLD |
| | | | | GVLNYPIYWQLLYAFESSSGSISD |
| | | | | LYNMIKSVASDCSDPTLLGNFIE |
| | | | | NHDNPRFAYYTSDYSEAKNVLS |
| | | | | YIFLSDGIPIVYAGEEQHYNGGD |
| | | | | VPYNREATWLSGYDTSAELYTW |
| | | | | IATTNAIRKLAIEADSDYITYAND |
| | | | | PFYTDSNTIAMRKGTSSQQVITV |
| | | | | LSNKGSSGSSYTLTLSGSGYTSGT |
| | | | | ELIEAYTCTSVTVDSNGDIPVPM |
| | | | | DSGLPRVFLPASSINSSSSSLCGG |
| | | | | SSTTTTTTTSTSPSTATSSSTATAT |
| | | | | TTCTTLPITFIESVTTTYGEEIYLS |
| | | | | GSITQLGDWDTSDAVLLSADNY |
| | | | | TSSNPEWYVEVSLPTGTSFEYKFI |
| | | | | KKESDGSVTWESDPNREYTVPEE |
| | | | | CGGVTVDDSWR |
| XP_ | Talaromyces | 75.5 | 25 | MVAIAKRAAFLASLFLGQSVVAL |
| 035340960.1 | rugulosus | | | TPAEWRSQSIYFLLTDRFGREDN |
| | | | | STTASCDLSERIYCGGSWQGIINH |
| | | | | LDYIQGMGFTAIWITPVTEQLPQ |
| | | | | DTGEGEAYHGYWQQEIYSVNSN |
| | | | | YGTAADLLALSEALHDRGMYLM |
| | | | | VDVVANHMGYAGAGDSVDYSV |
| | | | | FSPFDSSSYFHSYCLISDYSNQEN |
| | | | | VEDCWLGDTTVSLPDLNTDLTS |
| | | | | VQTLWYDWVADLVSNYSIDGLR |
| | | | | IDTVKHVQESFWPGYNSAAGVY |
| | | | | CVGEVFDGDPAYTCPYQNYLDA |
| | | | | VLNYPIYYQLLYAFESSSGSISGL |
| | | | | YDMISSVASDCADPTLLGNFIEN |
| | | | | HDNPRFASYTDDYSQAKNVISFV |
| | | | | FLSDGIPIIYAGQEQHYSGGSDPE |
| | | | | NREATWLSGYSTTAELYQFIATT |
| | | | | NKIRALAVSSDSSYITTKNDPFYT |
| | | | | DSNTIAMQKGSSGSQVVTVLSN |
| | | | | MGASGSSYTLTLSGSGYASGTEL |
| | | | | MEMYTCTSVTVDSSGNIAVPMA |
| | | | | SGLPRVFMLASSAGSLCGSSTTT |
| | | | | TTSKITSTTSTSSTSSTSTSCTQTT |
| | | | | DVPVLFKELVTTTYGEDIYISGSI |
| | | | | SELGDWDTANAVALSSSGYTSS |
| | | | | NPLWQVVVSIPAGTSFEYKFFET |
| | | | | GSSSSVTWESDPNRSYTVPTACS |
| | | | | GSTATVTATWR |
| KAF3403643.1 | Penicillium rolfsii | 77.5 | 26 | MKTLGLAAIFLAKAVAGLTPAE |
| | | | | WRSQSIYFLLTDRFGRTDNSTTA |
| | | | | TCNVSDRIYCGGSWQGIINHLDY |
| | | | | IQGMGFTAIWITPVTEQLSQDTG |
| | | | | DGEAYHGYWQQEIYNVNTNYG |
| | | | | TAADLLALSKALHSRGMYLMVD |
| | | | | VVANHMGYDGAGNTVDYSVEN |
| | | | | PFDSSSYFHSYCEITDYSNQTNVE |
| | | | | DCWLGDTTVSLPDLDTTLSSVQT |
| | | | | IWYNWVTGLVSNYSIDGLRIDTV |
| | | | | KHVQKSFWPGYNSAAGVYCMG |
| | | | | EVFDGDPAYTCAYQSYLDGILNY |
| | | | | PVYYQLLYAFESTSGSISNLYNMI |
| | | | | NSVASDCSDPTLLGNFIENHDNP |
| | | | | RFASYTSDYSQAKNVISFIFFSDG |
| | | | | IPIVYAGQEQHYSGGSDPANREA |
| | | | | TWLSGYDKTAQLYTYITTTNKIR |
| | | | | ALAISKDSAYITSKNTAFYSDSNT |
| | | | | IAMKKGSSGSQVITVLSNRGSSG |
| | | | | SSYTLSLSGSGYSSGKKLMEMYT |
| | | | | CTAVTVDSSGNIAVPMASGLPRV |
| | | | | YMLASSACSICSSTCSTTTSTASS |
| | | | | TSSSTTTASTTLKTTASSASTSCT |
| | | | | QATALPVLFKEIVTTSYGQNIYIS |
| | | | | GSISQLGSWDTSSAVALSADQYT |
| | | | | SSNHLWYVVVTIPVGTSFQYKFI |
| | | | | EETSGYSTITWESDPNRSYTVPTG |
| | | | | CVGSTATVTATWR |
| KAF7177279.1 | Aspergillus felis | 74.4 | 27 | MKWTSSLLPLSLSLLGQAAHALT |
| | | | | PAEWRSQSIYFLLTDRFGRQDNS |
| | | | | TTAACDVTQRLDYIQGMGFTAV |
| | | | | WITPVTEQFYEDTGDGTSYHGY |
| | | | | WQQNIYEVNSNYGTAQDLKSLA |
| | | | | NALHARGMYLMVDVVANHMG |
| | | | | YDGAGTSVDYSVFNPFNSSSYFH |
| | | | | PYCLISDYSNQTNVEDCWLGDTT |
| | | | | VSLPDLNTTETTVRTIWYDWVK |
| | | | | ALVANYSIDGLRIDTVKHVEKAF |
| | | | | WPDYNSAAGVYCVGEVFSGDPA |
| | | | | YTCPYQNYLDGVLNYPIYYQLL |
| | | | | YAFESTSGSISNLYNMISSVASDC |
| | | | | ADPTLLGNFIENHDNPRFASYTS |
| | | | | DYSQAKNVVSFIFFSDGIPIVYAG |
| | | | | QEQHYSGGADPANREAVWLSGY |
| | | | | STSATLYSWIASTNKIRKLAISKD |
| | | | | SAYVTSKNNPFYYDPNTLAMRK |
| | | | | GSTAGAQVITVLSNKGSSGSSYT |
| | | | | LSLSGTGYSAGATLVEMYTCTTL |
| | | | | TVDSSGNLAVPMASGLPRVLVP |
| | | | | ASWVSGSGLCGGSVSTTATTTTS |
| | | | | ATTTTTATTTTACTSATALPILFE |
| | | | | ELVTTTYGENIYLTGSISQLSNW |
| | | | | DTSSAIALSAGNYTSSNPEWYVT |
| | | | | VTLPIGTSFQYKFFKKESDGSITW |
| | | | | ESDPNRSYTVPTGCAGTTVTVSD |
| | | | | TWR |
| KAF7118914.1 | Aspergillus | 73.5 | 28 | MKWNLLLLPLSLSLLGQAAHAL |
| | hiratsukae | | | TPAEWRTQSIYFLLTDRFGREDN |
| | | | | STTAACDVTERLDYIQGMGFTAI |
| | | | | WINPVTEQFYEDTGDGTSYHGY |
| | | | | WQQNIYEANHNYGTAQDLKSLA |
| | | | | DALHARGMYLMVDVVANHMG |
| | | | | YDGAGNSIDYSVFTPFDSSSYFHP |
| | | | | YCLISDYSNQTNVEDCWLGDTT |
| | | | | VSLPDLDTTDAKVRTIWYDWVK |
| | | | | GLVANYSIDGLRIDTVKHVEKDF |
| | | | | WPGYNDAAGVYCVGEVFSGDPT |
| | | | | YTCPYQNYLDGVLNYPIYYELLY |
| | | | | AFESPRGSISNLYNMISSVASDCA |
| | | | | DPTLLGNFIENHDNPRFASYTSD |
| | | | | YSQAKNVISFMFFSDGIPIVYAGQ |
| | | | | EQHYSGGADPANREAVWLSGYS |
| | | | | TSATLYSWIASTNRIRKLAISKDT |
| | | | | AYITSKNNPFYYDSNTLAMRKGS |
| | | | | VAGAQVVTVLSNKGSSGSSYTLS |
| | | | | LSGTGYSAGASLVEMYTCTTLTV |
| | | | | DSSGNLAVPMASGLPRVLVPSS |
| | | | | WVSGSGLCGSGSTTTTTTTTTAT |
| | | | | ATTTACTSATALPILFEEILTTTFG |
| | | | | ENIYLSGSISQLGNWDTSSAIALS |
| | | | | ASKYTSSNPEWYVTVTVPVGTSF |
| | | | | QYKFFKNVSDGSIVWETDPNRSY |
| | | | | TVPSGCGGTTVTLSGTWR |
| XP_ | Aspergillus | 72.5 | 29 | MKWTSSLLPLSLSLLGQAAHALT |
| 043122688.1 | viridinutans | | | PAEWRSQSIYFLLTDRFGREDNS |
| | | | | TTAACDVTQRLDYIQGMGFTAI |
| | | | | WITPVTEQFYEDTGDGTSYHGY |
| | | | | WQQNIYEVNSNYGTAQDLKSLA |
| | | | | DALHARGMYLMVDVVANHMG |
| | | | | YDGAGNSVDYSVFTPFGSSSYFH |
| | | | | PYCLISDYSNQTNVEDCWLGDTT |
| | | | | VSLPDLDTTDTTVQTICTRTWAW |
| | | | | MILTVIVDGLRIDTVKHVEKDFW |
| | | | | PGYNDAAGVYCVGEVFSGDPTY |
| | | | | TCPYQNYLDGVLNYPIYYQLLY |
| | | | | AFESTSGRISNLYNMLSSVASDC |
| | | | | ADPTLLGNFIENHDNPRFASYTS |
| | | | | DYSQAKNVISFMFFSDGIPIVYAG |
| | | | | QEQHYSGGADPANREAVWLSGY |
| | | | | STSATLYSWIASTNKIRKLAISKD |
| | | | | SAYITSKNNPFYYDSNTLAMRKG |
| | | | | STAGSQVITVLSNKGFSGSSYTLS |
| | | | | LSGTGYSAGTTLVEMYTCTSLTV |
| | | | | DLSGNLAVPMASGLPLVLVPSS |
| | | | | WVSGSGLCGSSVSTTATATSTTK |
| | | | | TTIATTTAACTSATALPILFEELV |
| | | | | TTTYGENIYLTGSISQLGNWDTSS |
| | | | | AIALSASKYTSSNPEWYVTVTLP |
| | | | | VGTSLQYKFFKKESDGSIVWESD |
| | | | | PNRSYTVPTGCAGTTVTASDTW |
| | | | | R |
| RJE21582.1 | Aspergillus | 67.8 | 30 | MHLLISVVFLLLLQTVSSLSPAE |
| | sclerotialis | | | WRSQSIYFLLTDRFGRTDNSTTA |
| | | | | ECDVEKRLYCGGTWQGIINQLD |
| | | | | YIQGMGFTAIWITPVTEQLSQDT |
| | | | | GYGEAYHGYWQQKIYNLDSHLG |
| | | | | TADDLKALSKALHDRGMYLMV |
| | | | | DVVANHMGYDGPGNSVDYSVF |
| | | | | DPFDSSSYFHKYCLISNYDDQND |
| | | | | VQNCWLGDTTVSLPDLNTKDAE |
| | | | | VRELWYDWVTGLVSNYSIDGLR |
| | | | | IDTVKHVEKTFWEGYNQAAGVY |
| | | | | CVGEVFDGDPKYTCAYQSYLDA |
| | | | | VLNYPIYYPLLNAFKAPGGNMG |
| | | | | DLYNMVNSVASHCSDPTVLGNFI |
| | | | | ENHDNPRFASYTDDYSLAKNAIT |
| | | | | FMFLSDGIPIIYAGEEQHYSGGSD |
| | | | | PGNREAVWLSGYSTDAELYKFIA |
| | | | | TTNKIRSLVILKDNKYVTSKNVP |
| | | | | FYQDDNTFAMRKGSGNSQAITV |
| | | | | LSNAGSSSDSYSVTVKGAEYSSG |
| | | | | TQLIDLYSCAKVTVGDDGSISLQ |
| | | | | VESGLPKVLVPASYAGDALCGG |
| | | | | SSAPSIA |
| OQE16168.1 | Penicillium steckii | 68.9 | 31 | MYLIFAALLMLAKGISALSPAEW |
| | | | | RSQSIYFLLTDRFGREDNSTTASC |
| | | | | DVSNRLDYIQDMGFTAIWITPVT |
| | | | | EQLQGDTGYGEAYHGYWQQDI |
| | | | | YDVNENYGTAEDLLALSNALHD |
| | | | | RGMYLMVDVVANHMGYNGAG |
| | | | | NTVDYSVFNPFDSSSYFHSYCLIT |
| | | | | NYDNQDNVEDCWLGDTTVSLPD |
| | | | | LNTDLSSVQTIWYDWISDLVSNY |
| | | | | SIDGLRIDTVKHVQKSFWPSYNS |
| | | | | AAGVYCVGEIFDGNPAYTCDYQ |
| | | | | NYMDGVLNYPIYYQLLYAFQST |
| | | | | SGSISDLYNMIGTVASDCKDPTL |
| | | | | LGNFIENHDLPRFASFTSDYSLAK |
| | | | | NVLSFLFFSDGIPIVYAGQEQHYS |
| | | | | GKDDPANREATWLSGYSTSAEL |
| | | | | YKHISTTNKIRKLAASSDSSYITS |
| | | | | RVIIVSSKLDKPKGLTNSMQNTPF |
| | | | | YQDSHTIAMKKGSSGSQVITVLS |
| | | | | NYGSSGSSYTLTLGSSGYSSGTEL |
| | | | | MEMYTCTTATVDSSGNIPVPMV |
| | | | | SGLPRVLMLASVAKSSGPCASSG |
| | | | | SPTTSTASSSSTTMSACTKATALP |
| | | | | VLFKETVSTSYGEDIYISGSISRLG |
| | | | | SWDASNAVALSADQYTSSNPLW |
| | | | | YVSITLPVGTSFEYKFIKKTSGSS |
| | | | | TVTWESDPNRSYTVPTACSGSTA |
| | | | | TVTATWR |
| KAB8072940.1 | Aspergillus leporis | 65 | 32 | MVSWWSLVLCGLQVLGPAFAAT |
| | | | | PAEWRSQSIYFLLTDRFARTDGS |
| | | | | TTASCNTEDRKYCGGTWQGIIDK |
| | | | | LDYIQGMGFTAIWITPVTGQLPQ |
| | | | | HTAYGDAYHGYWQQDIYALNS |
| | | | | NYGTADDLKALASALHDRGMY |
| | | | | LMVDVVANHMGYDGAGDSVDY |
| | | | | SVFKPFNSKDYFHNFCLIQNYDD |
| | | | | QTQSENCWLGDNSVSLPDLDTT |
| | | | | REDVKGLWYDWVEALVSNYSID |
| | | | | GLRIDTVKHVQKDFWPGYNQAA |
| | | | | GVYCIGEVLNGDPAYTCPYQNV |
| | | | | MDGVLNYPMYYPLLNAFKSTSG |
| | | | | SISDLYNMINTVKSTCPDSTLLGT |
| | | | | FVENHDNPRFASYTNDMSLAKN |
| | | | | VAAFIILADGLPIIYAGQEQHYAG |
| | | | | GNDPANREATWFSGYSTTSEIYK |
| | | | | LIASSNAIRNHAISTDSGYLTYKN |
| | | | | WPVYQDASTIAMRKGTDGSQIIT |
| | | | | VLSNQGSSGGSYTLSLTGTGYTA |
| | | | | GQQLTEITTCSTVTVGLDGKVPL |
| | | | | PMEKGLPKVLYPSAKLSGSKVCS |
| | | | | SS |
| XP_ | Aspergillus tanneri | 66 | 33 | MKAGWLLSLASVALAATPADW |
| 033428176.1 | | | | RSQSIYFLLTDRFARSDGSTTASC |
| | | | | NTEDRKYCGGTWKGIIDQLDYIQ |
| | | | | GMGFTAIWITPVTGQLPQDTGYG |
| | | | | EAYHGYWQQDIYSLNDKYGTAD |
| | | | | DLKALADALHNRHMYLMVDVV |
| | | | | ANHMGYSGAGDSVDYSVFKPFN |
| | | | | SKDYFHPYCPIKDYNDQTQAENC |
| | | | | WLGDNTVSLPDLDTTKSEVKDL |
| | | | | WYKWVGDLVSNYSIDGLRIDTV |
| | | | | KHVQKDFWPGYNKAAGVYCVG |
| | | | | EVLNGDTAFTCAYQSVLDGVLN |
| | | | | YPMYYPLLNAFKSTSGNIGDLYN |
| | | | | MINNVKSNCPDSTLLGTFIENHD |
| | | | | NPRFASYTNDMSLAKNVAVFTIL |
| | | | | ADGIPIIYAGQEQHYAGGEDPAN |
| | | | | REATWLSGYSTKSELYTLIASSN |
| | | | | AIRNHAIAQDPKYVTYKNYPIYQ |
| | | | | DETTIAMRKGSDGSQVVTVLSN |
| | | | | KGSSGSSYTLSLGDTGYSSGQNV |
| | | | | TEIYTCETLTVGSDGKVSVPMNN |
| | | | | GLPRVLYPSEKLKGSSLCSE |
| KAE8144908.1 | Aspergillus | 63.4 | 34 | MVSWRSLLLGLQVVAPILAATPA |
| | avenaceus | | | DWRSQSIYFLLTDRFARTDGSTS |
| | | | | ATCNTEDRKYCGGTWKGIIDQL |
| | | | | DYIQGMGFTAIWITPVTSQLTQD |
| | | | | TAYGEAYHGYWQQDIYSLDSHL |
| | | | | GTADDLRALASALHERGMYLM |
| | | | | VDVVANHMGYDGAGDSVDYSV |
| | | | | FNPFNSKDYFHPFCLIQDYNDQT |
| | | | | QSENCWLGDNSVSLPDLDTTTQ |
| | | | | DVKNIWYDWVEELVSNYSIDGL |
| | | | | RVDTVKHVQKDFWPDYNKAAG |
| | | | | VYCIGEVLNGDPSYTCPYQEVM |
| | | | | DGVLNYPIYYPLLNAFKSTSGNM |
| | | | | NDLYNMINTVKSDCPDSTLLGTF |
| | | | | IENHDSPRFASYTNDMALAKNV |
| | | | | AAFIILADGIPIIYAGQEQHYAGG |
| | | | | NDPSNREATWLSGYSTTSDLYKL |
| | | | | IASSNAIRNHAVSTDKGYVTYKN |
| | | | | WPIYKDTTTIAMRKGTDGSQIITI |
| | | | | LSNKGASGDGYTLSLDGTGYDA |
| | | | | GTELTEILSCSTLTVGSDGKVPVP |
| | | | | MEKGLPRVLYPSAKLGDSKICT |
| XP_ | Aspergillus | 64 | 35 | MPIRWLSLILSCLAGTAFAATPEQ |
| 024681356.1 | novofumigatus IBT | | | WRSQSIYFLLTDRFARTDGSTTA |
| | 16806 | | | SCDTTARKYCGGTWQGIIKQLD |
| | | | | YIQGMGFTAIWITPVTEQLPQDTS |
| | | | | EGTAYHGYWQQDIYSVNSNYGT |
| | | | | ADDLKALGSALHDRGMYLMVD |
| | | | | VVANHMGYAGAGNSVDYSLFSP |
| | | | | FNSQTYFHPLCFISNYDNQTNVE |
| | | | | NCWLGDNVVPLADLDTTKSDVQ |
| | | | | KIWYNWVGSLVSNYSIDGLRIDT |
| | | | | VKHVQKDFWPGENDAAGVYCIG |
| | | | | EVFDGDPAYTCPYQDVLDGVLN |
| | | | | YPTYYPLLKAFQSTSGSMSDLYN |
| | | | | MINTVKSQCADSTLLGTFVENHD |
| | | | | TPGSHRKYTNDMALAKSAAAFII |
| | | | | LSDGIPIIYAGQEQHYSGGTDPAN |
| | | | | REAVWLSGYSKTSELYKLIASAN |
| | | | | AIRNHAISKDPEYVTYKNNPIYK |
| | | | | DTSTIAMRKGADGAQVITVLSNL |
| | | | | GASGSSYTLSLSGTGYDVGQQLT |
| | | | | EVFSCATVTVDSDGKVPVAMAS |
| | | | | GLPRAFYPTAGLNGSTICA |
| XP_ | Aspergillus nomiae | 65.8 | 36 | MVAWWSLFLYGLQAAAPALAA |
| 015402287.1 | NRRL 13137 | | | TPADWRSQSIYFLLTDRFARTDG |
| | | | | STTAACNTEDRKYCGGTWQGIID |
| | | | | KLDYIQGMGFTAIWITPVTGQLP |
| | | | | QNTAYGEAYHGYWQQDIYSLNE |
| | | | | NYGTPDDLKALASALHTRGMYL |
| | | | | MVDVVANHMGYDGAGASVDYS |
| | | | | VFKPFNSQEYFHPFCLIQNYNDQ |
| | | | | TQSEDCWLGDNSVSLPDLDTTK |
| | | | | DEVKNEWYEWVGSLVSNYSIDG |
| | | | | LRVDTVKHVQKDFWPGYNKAA |
| | | | | GVYCIGEVLNGDPAYTCPYQDV |
| | | | | MDGVLNYPIYYPLLNAFKSTSGS |
| | | | | MDDLYNMINTVKSDCPDSTLLG |
| | | | | TFVENHDNPRFASYTNDIALAKN |
| | | | | VAAFIILNDGIPIIYAGQEQHFAG |
| | | | | GNDPANREATWLSGFSTDSEIYK |
| | | | | LIASANAIRSHAISIDTGFVTYKN |
| | | | | WPIYKDTTTIAMRKGTDGSQVV |
| | | | | TILSNKGASGDSYTLSLGNTGYT |
| | | | | AGQKLTEVIGCTTVTVGSDGTVP |
| | | | | VPMAGGLPRILYPTEKLGDSKICS |
| | | | | SA |
| KAB8244571.1 | Aspergillus flavus | 63.5 | 37 | MVSWWSFLCGLQVFGLALAATP |
| | | | | AEWRSQSIYFLLTDRFARTDGST |
| | | | | TASCNTEDRKYCGGTWQGIIDKL |
| | | | | DYIQGMGFTAIWITPVTGQLPQH |
| | | | | TAYGDAYHGYWQQDIYTLNRN |
| | | | | YGTADDLKALASALHDRGMYL |
| | | | | MIDVVVNHMGYDGAGDSVNYS |
| | | | | VFKPFNTKDYFHNFCFIQNFDDQ |
| | | | | TQSENCWLGDNRVSLPDLDTTR |
| | | | | EDVKGLWYDWVEAFVSNYSIDG |
| | | | | LRVDTVKHVQKDFWRGYNEAA |
| | | | | GVYCIGEVENGDPAYTCPYQNIM |
| | | | | DGVLNYPMYYPLLNAFKSTSGSI |
| | | | | SNLYNMINTVKSTCPDSTLLGTF |
| | | | | VENHDNPRFASYTNDMSLAKNV |
| | | | | AAFIILADGLPIIYAGQEQHYAGG |
| | | | | NDPANRDATWFSGYSTTSELYK |
| | | | | LIASLNAIRRHAINTDSGYLSYKN |
| | | | | WPIYQDASTIAMRKGTDGSQIIT |
| | | | | VLSNQGSSGGSYTLSLTGTGYTA |
| | | | | GEQLHEIINCSTVTVGSDGKVLLP |
| | | | | MEKGLPRVLYPSAKLNGSKVCS |
| | | | | SS |
| GFF61057.1 | Aspergillus | 65.1 | 38 | MKLLTIALTLPLLGKSVLALNAA |
| | udagawae | | | EWRSQCIYFLMTDRFARTDGSTT |
| | | | | APCDVGQRGYCGGSWKGIIDHL |
| | | | | AYIQGMGFTAIWITPIVEQIPQTT |
| | | | | TEGTGFHGYWPQNIYSVNSHFGT |
| | | | | ADDIRALSKALHDRGMYLMMD |
| | | | | GVANHMGFNGPGASNGFSTFTP |
| | | | | FNSASYFHSYCPINNYNDQWQV |
| | | | | ENCYLRDSVVSLTDLNTQSSAVR |
| | | | | GIWYDWVKDLVANYSVDGLRID |
| | | | | TAKHVEKDFWSGYTQAAGVYS |
| | | | | VGEILHGDPAYTCPYQGYMDGV |
| | | | | MNYPIYYQLVNAFKSSSGSISSLS |
| | | | | NMISSVASKCKDPTLLGNFIENH |
| | | | | DNPRFPSYTSDISQAKSVIAYVFL |
| | | | | TDGIPIIYSGQEQHLSGGADPYNR |
| | | | | EALWLSGYSTNSELYKFIAMTNK |
| | | | | IRRLAISKDLNYLPARNNPFYTDS |
| | | | | NTIAMKKGSGGSNVITVLTNSGS |
| | | | | NAGSYTLNLNSHGYSSGSSLIEL |
| | | | | YTCSSVQVDSNGNLPVPMSSGLP |
| | | | | RVLVPSAWVPGSGLCGASSTTSL |
| | | | | ATPTVTSPSPGTCAPSTAIPVIFKE |
| | | | | RVATSYGENIFLSGSIDQLGNWD |
| | | | | TSKAVALSASGYTSSNPVWSVKL |
| | | | | DLHAGTYFQYKFIKKGQDGSVM |
| | | | | WESDPNRSYTLPSGCVETAISIAD |
| | | | | SWR |
| PYH96808.1 | Aspergillus | 64.1 | 39 | MMEILILFALVATVLGATPADWR |
| | ellipticus CBS | | | SQSIYFLLTDRFARTDNSTTASCN |
| | 707.79 | | | TGARQYCGGTWQGIINQLDYIQG |
| | | | | MGFTAIWITPVTGQIPQETGYGE |
| | | | | AYHGYWQQDAYALNSHYGTAD |
| | | | | DLKKLVSALHARGMYLMVDVV |
| | | | | ANHMAYNGSADTTDYSAYNPEN |
| | | | | SKEYFHKICSIEDNNNQTQTEDC |
| | | | | WLADSIVSLPDLNTTQTDVKNM |
| | | | | WYDWIGSLVSNYSIDGLRIDTVK |
| | | | | NVQQNFWPDYNTAAGVYCVGE |
| | | | | VFDGDAEYTCPYQNYLDGVLNY |
| | | | | PMYYPILRAFQSTSGSISDLYNMI |
| | | | | NTVKSTCKDSTLLGTFVENHDNP |
| | | | | RFANYTSDMSLAKNAAAFTMLA |
| | | | | DGIPIIYAGQEQHYHGGNDPYNR |
| | | | | EATWLAKYSTTSPLYELIATSNRI |
| | | | | RSHAISQDSGYITYKNYPIYQDNS |
| | | | | TLAMRKGYNGSQTITVLSNLGAS |
| | | | | GKTYNLSLPGTGYTANQQLMEI |
| | | | | YTCTNITVDSSGSVSVPMASGLP |
| | | | | RVLYPADKLNGSLLCNSTTSSAT |
| | | | | SLSPLSNSAIPIYLTLAAFVVQLS |
| | | | | QL |
| RMJ21212.1 | Aspergillus sp. | 63.3 | 40 | MKPFTYAIAMFLAQTVAALSPAE |
| | HF37 | | | WRGQSIYFLLTDRFARADNSTTA |
| | | | | PCDVTDRVYCGGSWQGIINQLD |
| | | | | YIQGMGFTAIWVSPVTEQLPQDT |
| | | | | EYGEAYHGYWQQDIYNLNKHFG |
| | | | | TPDDLNALADALHERGMYLMV |
| | | | | DVVPNHMGYDGPGDSVDYGVF |
| | | | | EPFSSSSYFHPFCFIENYDNQDNV |
| | | | | EQCWLGDSKVPLPDLDTSREDV |
| | | | | QNIWYDWVSGLVSNYSVDGLRL |
| | | | | DTAKHVQKNFWPDYNKAAGVY |
| | | | | CVGEVLSGDPKYTCPYQEYMDA |
| | | | | VLNYPIYYQLLDAFKSTNGPTDK |
| | | | | VYNMISRVASSCRIPTLLGNFIE |
| | | | | NHDAPRFASYTQDLALAKNAIAF |
| | | | | LFLTDGIPIVYAGQEQHYSGGDD |
| | | | | PFNRGAIWLSGYSTEAALYRFIG |
| | | | | DTNKVRSQAIAMDSAYLTSQNEP |
| | | | | FYHDDSTIAMRKGPKGSEVLTVL |
| | | | | TNSGSKGSYTVSIDAGYGSGTEL |
| | | | | VDLYSCNTATVGSDGTVSVKISA |
| | | | | GMPQALVPASAAGNSALCGNSS |
| | | | | K |
| XP_ | Talaromyces | 66.7 | 41 | MKFSNVSLVWSALASVALGLTA |
| 040731029.1 | amestolkiae | | | AEWRSQSIYFLLTDRFALTSNSTT |
| | | | | ASCDVADGLYCGGSWQGVINHL |
| | | | | DYIQGMGFTAIWITPVTENFEGD |
| | | | | TSDGEAYHGYWQQNAYATNSH |
| | | | | YGASSDLLKLSEALHARGMYLM |
| | | | | VDIVVNNMAYDGAGTSVDYSIF |
| | | | | NPFPSKSYYHSYCLIDYSTYNAT |
| | | | | DWNVCWEGDNIVSLPDIDTTQT |
| | | | | YVKDTWGTWVESFVANYSIDGL |
| | | | | RIDSAGHIQQDFFTAFEESSGVYC |
| | | | | IGEVDYGDPAVVCPYQNYLSGV |
| | | | | LNYPMYYQLLYAFESSSGSISNL |
| | | | | YNMINTVKSDCADTSLLGNFIEN |
| | | | | HDNPRFAYYTSDYSEAKNVISFIF |
| | | | | LTDGIPIL YYGQEQHYNGGNIPL |
| | | | | NREALWTSDYSTTAQLYTHTKT |
| | | | | SNAIRSLAITKDSAFLTYKNTPIY |
| | | | | QDSNTIAMRKGTTGLQLVTVLSN |
| | | | | LGASGSSSTLTLSGSGYTSGTVV |
| | | | | TELYTCTNVTVSSSGTIAVPMAS |
| | | | | GSPRAFLPWSSVSGSSLCSGSGSS |
| | | | | CTAASTVAVTFEEVVTTTYGQEV |
| | | | | YISGSISQLGNWSTSSAVLLSASQ |
| | | | | YTSSDPVWTVTISLPAGESFQYK |
| | | | | FIIVNTDGSITWESDPNRSYTVPT |
| | | | | GCAGLTATVDDTWR |
| XP_ | Talaromyces | 63.3 | 42 | MKLSLLGGTTAVALLSGVANAA |
| 035350392.1 | rugulosus | | | TPDDWRSQSIYFLLTDRFARADN |
| | | | | STTATCNTEDGIYCGGSWQGVIN |
| | | | | QLDYIQGMGFTAIWISPVTGQLT |
| | | | | EVTSDGSSYHGYWQQDIYALNE |
| | | | | NFGTSDDLNDLASALHDRGMYL |
| | | | | MVDVVANHMGYAGDPTTVDYS |
| | | | | VFNPFNSQDYFHPYCALTDYTNQ |
| | | | | TMTEECWMGDDTVCLPDLDTES |
| | | | | TSVQDIWFSWIGDLVANYSIDGI |
| | | | | RIDTVAEVSKDFWATYNEKAGV |
| | | | | YAVGEVDNGDVTYACPYQTALD |
| | | | | GILNYPTYFPLVRAFESSSGSISEL |
| | | | | ADMINSVKSDCTDTNLLGSFSEN |
| | | | | HDNPRFASYTQDLALAKNVLTY |
| | | | | TILADGIPIIYAGQEQHYDGAEDP |
| | | | | ANREATWLSGYNTTSELYTWVA |
| | | | | KMNKIRSFAIKSDDGYTTYNNYP |
| | | | | IYQDDNNLGMRKGSNGSQIITVV |
| | | | | SNLGADGGQTTLTVSGGGYTAG |
| | | | | TVLTELITCTSVTVGDGGDISVSL |
| | | | | DSGLPSVLYPASKLNGTGSPC |
| AYN78123.1 | Thermoascus | 63.7 | 43 | MHLRSFSILPALVVGTALAATPA |
| | crustaceus | | | QWRSQSVYFLLTDRFARTDGSTT |
| | | | | AACDTDARAYCGGTWQGIIDHL |
| | | | | DYIQGMGFTAIWITPVTENLPQD |
| | | | | TGDGTSYHGYWQQDVYSLNSN |
| | | | | YGTPDDLRALSSALHDRGMYLM |
| | | | | VDVVANHMGYAGPGSSVDYTV |
| | | | | FTPFNDQKYFHPYCSISNYDDQS |
| | | | | NVEDCWLGDSTVSLPDLDTTRS |
| | | | | DVQDMWYSWVKGLVANYSVD |
| | | | | GLRIDTVKHVQKDFWPGYNDAA |
| | | | | GVYCVGEVFDGDPSSTCDYQNY |
| | | | | LDGVLNYPMYYPLLRAFSSTSGS |
| | | | | ISDLYNMINTVKSECADSTLLGTF |
| | | | | VENHDNPRFASYTSDISLAKNAL |
| | | | | AFTILSDGIPIIYAGQEQHYSGGN |
| | | | | DPANREAVWLSGYSTTSELYKFI |
| | | | | AVSNQIRNHAISVDGDDYLTYKT |
| | | | | YPIYQDTTTLAVRKGSLITVLSNL |
| | | | | GSSGSSYTLSLGGTGYSSGQELM |
| | | | | EIYSCTTVTADSSGNIAVPMGSG |
| | | | | LPKAFYPTANLGGSGICGK |
| XP_ | Aspergillus | 62.9 | 44 | MRSTLLLSLAATALAATPAQWR |
| 041552793.1 | puulaauensis | | | SQSIYFLLTDRFARTDNSTTAECD |
| | | | | TSAGRYCGGSWQGIINQLDYIQG |
| | | | | MGFTAIWITPVTAQVEDSSSGDA |
| | | | | YHGYWQQDLYFLNSQLGTKDDL |
| | | | | LALSDALHERDMYLMVDVVAN |
| | | | | HMGYDGAADSVDYSVFNPFNSQ |
| | | | | DYFHSPCSIDDYDDQTQVEECWL |
| | | | | STSAVSLPDVDTTRDDVKTLWY |
| | | | | DWVEALVSNYSIDGLRVDTVRH |
| | | | | VQKDFWADENDAAGVYCVGEV |
| | | | | LQGDPEYTCAYQELMDGVLNYP |
| | | | | IYYPLLRAFSSTSGSISELYDMINS |
| | | | | VKSTCADSTLLGSFIENHDNPRF |
| | | | | ASETDDISLAKNVAAFVILSDGIPI |
| | | | | IYAGQEQHYAGGEDPENREATW |
| | | | | LSGFDTSSELYKLIAASNAIRTHA |
| | | | | IGQDDEWITYKNYPIYQDTSSLA |
| | | | | MRKGNNGTQVVTVLTNAGAGG |
| | | | | NSYTLSLPDTGYSAGAALTEVLS |
| | | | | CTDITVSDNGEVPVPMESGLPRV |
| | | | | LYPTAKLEGSGICQ |
| XP_659622.1 | Aspergillus | 61.2 | 45 | MRSPLFLSFAATVLAATPAEWRS |
| | nidulans FGSC A4 | | | QSIYFLLTDRFARTDNSTTAECDT |
| | | | | SAKYCGGTWQGIINQLDYIQGM |
| | | | | GFTAIWITPVTANLEDGQHGEAY |
| | | | | HGYWQQDIYALNPHFGTQDDLR |
| | | | | ALSDALHDRGMYLMVDVVANH |
| | | | | FGYDAPAASVDYSAFNPENSAD |
| | | | | YFHTPCDITDYDNQTQVEDCWL |
| | | | | YTDAVSLPDVDTTNEEVKEIWY |
| | | | | DWVGDLVSDYSIDGLRIDTARH |
| | | | | VQKDFWRDYNDAAGVYCVGEV |
| | | | | FQGDPDYTCGYQEVMDGVLNYP |
| | | | | IYYPLLRAFSSTSGSLSDLANMIE |
| | | | | TVKYTCSDATLLGNFIENHDNPR |
| | | | | FASYTDDISLAKNVAAFVILSDGI |
| | | | | PIIYAGQEQHYSGAGDPANREAT |
| | | | | WLSGYDSTSELYQFISKTNQIRN |
| | | | | HAIWQNETYLSYKNYAIYNENN |
| | | | | VLAMRKGFDGSQIITILTNAGAD |
| | | | | AGSSTVSVPNTGFTAGAAVTEIY |
| | | | | TCEDITVSGSGEVSVPMESGLPR |
| | | | | VLYPKAKLEGSGICGL |
| XP_ | Talaromyces | 65.3 | 46 | MKFSLAAIAAIASLLAPVVKAAT |
| 020119254.1 | atroroseus | | | PDDWRSRSIYFLLTDRFARTDNS |
| | | | | TTATCETLYGVCSKTTVEDHGRL |
| | | | | DYIQDMGFTAIWISPVTEQLPQST |
| | | | | ADGEAYHGYWQQDIYALNSNFG |
| | | | | TAEDLQNLATALHDRGMYLMV |
| | | | | DVVANHFGYAGAGDDVDYAIFN |
| | | | | PFNSESYFHPFCLITDYSNETMVE |
| | | | | ECWEGDNIVSLPDLDTESTAVQN |
| | | | | ICLTRSYDNEDEATANGNYEPVD |
| | | | | GLRLDSVMEVQKDFWPDWNSA |
| | | | | SGVYCVGEVDDGDPTFTCPYQK |
| | | | | YLDGVLNYPTYFPLVRAFESSSG |
| | | | | SISELYDMINEVKSDCVDSNLLG |
| | | | | SFSENHDNPRFASYTSDFSLAKN |
| | | | | ALAFTILSDGIPIIYAGQEQHYDG |
| | | | | GNNPYNREATWLSGYDTSAELY |
| | | | | TFIAVLNQIRNYAIQWDNGYTTY |
| | | | | KTYPIYQDDNNLAIRKGSDESQII |
| | | | | TVLSNVGTDADSYSLTISGTGYT |
| | | | | AGEVLTDLISCTNVTVDDSGDVS |
| | | | | VTMTGGLPSVLYPTYKLVYDGH |
| | | | | PC |
| KGO67837.1 | Penicillium | 61.6 | 47 | MILAQFACLAGLIGTAIAATPAE |
| | italicum | | | WRSRSIYFMLTDRFARTDGSTTA |
| | | | | ACNTGDRKYCGGTWQGIIDKLD |
| | | | | YIQGMGFTAIWITPVTSQLTVDTP |
| | | | | YGEPYHGYWQQDIYALNSNYGT |
| | | | | ADDLKALAAALHERDMYLMVD |
| | | | | VVANHMGYNGAGADVDYTKEN |
| | | | | PFNDAKYFHPYCPITDYDNDTM |
| | | | | AQNCWLGDDKVSLPDLDTQSME |
| | | | | VQDIWYDWVRSLVSNYSKTDEY |
| | | | | KVDGLRIDTVKHVQKDFWPGYN |
| | | | | KAAGVYCVGEVFDGDVDYTCPY |
| | | | | QEVMDGVLNYPVYYPLLKAFQS |
| | | | | TSGSMADLFNMINRVKSTCKDST |
| | | | | LLGNFLENHDNPRFASVTDDIAL |
| | | | | AKNAATFTIMADGIPIVYAGQEQ |
| | | | | HYSGGEDPANREALWLSGENTD |
| | | | | SELYKLIATVNGARNQAIAKSTN |
| | | | | YTIYQNYPIYKDSSTIAMRKGYD |
| | | | | SGQTITVLTNLGAGGKDYSVSIP |
| | | | | DTGFAAGAKLTEVISCVSVTAGE |
| | | | | SGEVSVPMAGGAPRILLPTSLLE |
| | | | | GSTLCSS |

SEQUENCES

ATGAGGGTTGCTACATTTTTAGGAACTCTACTAGCCAGTCAGGTTACTGTGTCTGCTCTATCTCCTGCT
GGTTGGCGGGAGCAGTCCATCTATCAGGTTGTCACAGATCGCTTCGCGCTCACCTCTGGAGGGTCTTCA
CCATCGTGCACTTCAGACTTCCAACTCTCAATCTTCTGCAATGGAACATTTCGAGGAATAATTGACAAA
TTGGATTACATCCAAGGCATGGGCTTCACCGCCGTGAGTTATCCGCAGAGATAGTGTTTAGAAATATA
TTAATATTGCTGTTAGATGTGGATCTCCCCAATCGTCGAAAATATCAAAGGCGGCACACCAGATGGTT
ATACTGAGGATGGTTCTGCTTATCATGGCTACTGGGCGCAAGATATATATGCTATCAACCCGCACTTTG
GTACCCCCGAAGATCTTGTGGCGTTGGCAGAGGCTTTGCACAATCGCGGAATGGTACGAAGTCAACGC
GTTTTAATCAATTGAACTTATACTAATTAAGCAATTAGTATCTCATGGTTGACATCGTAGTCAACCACA
TGGGATATTATTGTGGCAAAGCAGAAGACGGTCATTGTGGACCGCAGGGCACGATTGACTATTCAATT
TATAACCCGTTCAATTCAGAGAAATATTTCCATCCTTTTTGTGAGATCAACTACAACAATGCAACGAGT
ATCTTGGTGGTACGTCAGAAAGCAATATGTATAAACACCACAAGTGAAGCTTACCGTTGCAGTGTTGG
GAAGGTGACGAGATCGTACCGCTACCGGATTTGCGCACCGAGAATACCACTGTGCAATCCAGTGCGTG
ATCTGCATCAGATCCATTGAAAATATGCTAAAAGTTTTCCAGTGTTCGATACGTGGATTACCGAACTTG
TAAAGAATTATACAAGTAAGTCCCATCGAAACCATCATTAGCAGTGACATGGGAAGATGATAAGGAC
CATTTCACCTCGAAAGAAATCTCATTTGAAACCCATACTAGTCGACACTTGTGAGCAGGATCAGCAGC
TAACGAAGGTGAAGTTGATGGACTCCGTATCGATAGTTTGCAGCAATCTGGATCTTTCTTCTTCCCGTC
CTTTTATAAGGCCGCGGGAGAGCTATACATGGTTGGAGAAGTCTTTCAGGAATACCCAGCTAAAGTTT
GTCCATATCAGCAAGATGGGATGCCTGGTGTCTTGAACTATCCAATGTGCGTCCTTTGGCCCGACTTTG
CCTTCTTAGCGACTCTTGGTAACAGTCTGTAGGTTTTATTACATCACCGATGCCTTCCAGGATAGCACG
GGTAGCATGAGTGGGCTTGCCGAAGGCATCTCTTATATGCAAGGCAATTGCAGTGACACAACATTGCT
AGGAAGTTTCCTCGAGAATCAGGATAATCCCCGCTTTCCATCGTAAGCACTCAAGTTTCTATAATCAAG
GTTTAAGCTCTGACTAGCACACAGATATACCGAAGACATTACTCGTGCTAAGAATGCCATTGCGTTCA
CTATGCTTCAAGATGGAATTCCTATCATCTACTATGGCCAAGAGCAGCATCTCAACGGCTCTGGCGTGC
CTCAAAATCGTGAAGCGCTATGGTCTTCAGGAGGCTACAATACTGCATCTATGCTCTATAACATGATA
AAAGAAGTTAATGCTATCAGAACGCAGGCCATTCATGTTTCTCAAAGTTTCGTCACAGAGAAAATAAC
CACACCTTACTACGACAACCACACGATCGTTACCCGCAAAGGCGAGACCGGCTCGCAAATAATCGGA
GTATATTCCAATCTCGGCGAAAATGGTGCTTATTATAATTTATCACTCAAATCAGCTGTTACTGGTTTT
GGGAACGAAGAAAAAATCATGGAGATCCTGACATGCACCTTGCTGACGACCAGTTCTGATGGTGAGA
TTATCGTGCCAATGGGTGAGGGACAACCTAAAATACTATTTCCGTTCGCAAGACTCAGTGGCAGCTCG
ATTTGTAAGAGTTATAAGAGTAAGCTAATCGATATATCACTTTTACTTGTGTAGATTCAAGCTGATCAA
AGATGCTCCGTAGCAATCAGCCCAGTCGGAATAGACTGTGAGCCAGCTACAATTACTTTTGACATCCT
GGTAAACACTACCTACGGCGAGTCTGTCGTTGTTGCTGGTGATATACCTGAATTAGGAGATTGGAACG
TGCCCGATGGTCTTTATCTCAATGCTAGCAAGTATACGACTGAAAATCCTCTTTGGACCGGAACTGCGA
CAGGCATTGATCCCCAGGAAGTTTTCCAATGGAAACCCGTTGTCATACTAACAAATGGGAGCTATGTA
TATGCCCCTGGTGACAATATTGAAACGGTCGCTCCTGGGCCAGGTTGTAATCCGACGGCGACGATAAA
ATACGTTTTTTCTGGCCAGTGA (SEQ ID NO: 1)
ATGCGAGTCGCTACCTTTCTCGGCACCCTCCTCGCCAGCCAGGTCACCGTCAGCGCTCTTAGCCCTGCC
GGCTGGCGCGAGCAGAGCATCTACCAGGTCGTCACCGACCGCTTCGCCCTCACTTCTGGCGGCAGCAG
CCCTAGCTGCACCAGCGACTTCCAGCTCAGCATCTTCTGCAACGGCACCTTCCGCGGCATCATCGACA
AGCTCGACTACATCCAGGGCATGGGCTTCACCGCCATGTGGATCAGCCCCATCGTCGAGAACATCAAG
GGCGGCACCCCCGACGGCTACACCGAGGATGGCTCTGCCTACCACGGCTACTGGGCCCAGGACATCTA
CGCCATCAACCCCCACTTCGGCACGCCCGAGGATCTCGTCGCTCTCGCCGAGGCCCTTCACAACCGCG
GCATGTACCTCATGGTCGACATCGTCGTCAACCACATGGGCTACTACTGCGGCAAGGCCGAGGACGGC
CATTGCGGCCCTCAGGGCACCATCGACTACAGCATCTACAACCCCTTCAACAGCGAGAAGTACTTCCA
CCCCTTCTGCGAGATCAACTACAACAACGCCACCAGCATCCTCGTCTGCTGGGAGGGCGACGAGATCG
TCCCTCTCCCTGACCTCCGCACCGAGAACACCACCGTCCAGAGCATGTTCGACACCTGGATCACCGAG
CTGGTCAAGAACTACACCATCGACGGCCTCCGCATCGACAGCCTCCAGCAGAGCGGCTCGTTCTTCTT
CCCGTCCTTCTACAAGGCCGCTGGCGAGCTGTACATGGTCGGCGAGGTCTTTCAGGAGTACCCCGCCA
AGGTCTGCCCCTACCAGCAGGACGGCATGCCCGGCGTCCTCAACTACCCCATGTTCTACTACATCACC
GACGCCTTCCAGGACAGCACCGGCAGCATGTCTGGCCTCGCCGAGGGCATCAGCTACATGCAGGGCA
ACTGCAGCGACACGACCCTGCTCGGCAGCTTCCTCGAGAACCAGGACAACCCCCGCTTCCCCAGCTAC
ACTGAGGACATCACCCGCGCCAAGAACGCCATTGCCTTCACCATGCTCCAGGACGGCATCCCCATCAT
CTACTACGGCCAGGAGCAGCACCTCAACGGCAGCGGCGTCCCTCAGAACCGCGAGGCTCTCTGGTCTA
GCGGCGGCTACAACACCGCCAGCATGCTCTACAACATGATCAAGGAGGTCAACGCCATCCGCACCCA
GGCCATCCACGTCAGCCAGAGCTTCGTCACCGAGAAGATCACCACCCCCTACTACGACAACCACACCA
TCGTCACCCGCAAGGGCGAGACTGGCAGCCAGATCATCGGCGTCTACAGCAACCTCGGCGAGAACGG
CGCCTACTACAACCTCAGCCTCAAGAGCGCCGTCACCGGCTTCGGCAACGAGGAGAAGATCATGGAG
ATCCTGACCTGCACCCTCCTCACCACCTCCAGCGACGGCGAGATCATCGTCCCCATGGGCGAGGGCCA
GCCCAAGATCCTCTTCCCGTTTGCCCGCCTCAGCGGCAGCAGCATCTGCAAGAGCTACAAGACCATCA
GCCCCGTCGGCATCGACTGCGAGCCCGCCACCATCACCTTCGACATCCTGGTCAACACCACCTACGGC
GAGAGCGTCGTCGTGGCCGGCGACATTCCTGAGCTGGGCGACTGGAACGTCCCCGACGGCCTCTACCT
CAACGCCAGCAAGTACACCACCGAGAACCCCCTCTGGACCGGCACCGCCACCGGCATTGACCCCCAG
GAGGTGTTCCAGTGGAAGCCCGTCGTCATCCTCACCAACGGCAGCTACGTCTACGCCCCTGGCGACAA
CATCGAGACTGTCGCCCCTGGCCCTGGCTGCAACCCCACCGCCACGATCAAGTACGTCTTTAGCGGCC
AG (SEQ ID NO: 2)
MRVATFLGTLLASQVTVSALSPAGWREQSIYQVVTDRFALTSGGSSPSCTSDFQLSIFCNGTFRGIIDKLDYI
QGMGFTAMWISPIVENIKGGTPDGYTEDGSAYHGYWAQDIYAINPHFGTPEDLVALAEALHNRGMYLMV
DIVVNHMGYYCGKAEDGHCGPQGTIDYSIYNPENSEKYFHPFCEINYNNATSILVCWEGDEIVPLPDLRTEN
TTVQSMFDTWITELVKNYTIDGLRIDSLQQSGSFFFPSFYKAAGELYMVGEVFQEYPAKVCPYQQDGMPGV
LNYPMFYYITDAFQDSTGSMSGLAEGISYMQGNCSDTTLLGSFLENQDNPRFPSYTEDITRAKNAIAFTMLQ
DGIPIIYYGQEQHLNGSGVPQNREALWSSGGYNTASMLYNMIKEVNAIRTQAIHVSQSFVTEKITTPYYDNH
TIVTRKGETGSQIIGVYSNLGENGAYYNLSLKSAVTGFGNEEKIMEILTCTLLTTSSDGEIIVPMGEGQPKILF
PFARLSGSSICKSYKTISPVGIDCEPATITFDILVNTTYGESVVVAGDIPELGDWNVPDGLYLNASKYTTENPL
WTGTATGIDPQEVFQWKPVVILTNGSYVYAPGDNIETVAPGPGCNPTATIKYVESGQ (SEQ ID NO: 3)
MSFRSLLALSGLVCTGLANVISKRATWDSWLSNEATVARTAILNNIGADGAWVSGADSGIVVASPSTDNPD
YFYTWTRDSGLVLKTLVDLFRNGDTSLLSTIENYISAQAIVQGISNPSGDLSSGAGLGEPKFNVDETAYTGS
WGRPQRDGPALRATAMIGFGQWLLDNGYTSTATDIVWPLVRNDLSYVAQYWNQTGYDLWEVNGSSFFTI
AVQHRALVEGSAFATAVGSSCSWCDSQAPEILCYLQSFWTGSFILANFDSSRSAKDANTLLLGSIHTFDPEA
ACDDSTFQPCSPRALANHKEVVDSFRSIYTLNDGLSDSEAVAVGRYPEDTYYNGNPWFLCTLAAAEQLYD
ALYQWDKQGSLEVTDVSLDFFKALYSDATGTYSSSSSTYSSIVDAVKTFADGFVSIVETHAASNGSMSEQY
DKSDGEQLSARDLTWSYAALLTANNRRNVVPSASWGETSASSVPGTCAATSAIGTYSSVTVTSWPSIVATG
GTTTTATPTGSGSVTSTSKTTATASKTSTSTSSTSCTTPTAVAVTFDLTATTTYGENIYLVGSISQLGDWETS
DGIALSADKYTSSDPLWYVTVTLPAGESFEYKFIRIESDDSVEWESDPNREYTVPQACGTSTATVTDTWR
(SEQ ID NO: 5)
QTTSVTSYIASESPIAKAGVLANIGADGSLSSGAYSGIVIASPSTVNPNYLYTWTRDSSLTFMELINQYIYGED
DTLRTLIDEFVSAEATLQQVTNPSGTVSTGGLGEPKFNINETAFTGPWGRPQRDGPALRATAIMAYATYLY
ENGNTSYVTDTLWPIIELDLGYVAEYWNESTFDLWEEIDSSSFFTTAVQHRALRAGVTFANLIGETSDVSNY
QENADDLLCFLQSYWNPTGSYVTANTGGGRSGKDANTLLASIHTFDPDAGCNATTFQPCSDKALSNHKVY
VDSFRSLYAINDDISSDAAVATGRYPEDVYYNGNPWYLCTLAAAEQLYDSLIVWKAQGYIEVTSLSLAFFQ
QFDASVSAGTYDSSSDTYTTLLDAVQTYADGFVLMVAQYTPANGSLSEQYAKADGSPTSAYDLTWSFAA
ALTAFAARDGKTYGSWGAADLSSTCSGSTDTVAVTFEVQYDTQYGENLYITGSVSQLEDWSADDALIMSS
ADYPTWSITVDLPPSTLIQYKYLTKYNGDVTWEDDPNNEITTPASGSYTQVDSWH (SEQ ID NO: 7)
MRVATFLGTLLASQVTVSALSPAGWREQSIYQVVTDRFALTSGGSSPSCTSDFQLSIFCNGTFRGIIDKLDYI
QGMGFTAMWISPIVENIKGGTPDGYTEDGSAYHGYWAQDIYAINPHFGTPEDLVALAEALHNRGMYLMV
DIVVNHMGYYCGKAEDGHCGPQGTIDYSIYNPFNSEKYFHPFCEINYNNATSILVCWEGDEIVPLPDLRTEN
TTVQSMFDTWITELVKNYTIDGLRIDSLQQSGSFFFPSFYKAAGELYMVGEVFQEYPAKVCPYQQDGMPGV
LNYPMFYYITDAFQDSTGSMSGLAEGISYMQGNCSDTTLLGSFLENQDNPRFPSYTEDITRAKNAIAFTMLQ
DGIPIIYYGQEQHLNGSGVPQNREALWSSGGYNTASMLYNMIKEVNAIRTQAIHVSQSFVTEKITTPYYDNH
TIVTRKGETGSQIIGVYSNLGENGAYYNLSLKSAVTGFGNEEKIMEILTCTLLTTSSDGEIIVPMGEGQPKILF
PFARLSGSSICKSYKTISPVGIDCEPATITFDILVNTTYGESVVVAGDIPELGDWNVPDGLYLNASKYTTENPL
WTGTATGIDPQEVFQWKPVVILTNGSYVYAPGDNIETVAPGPGCNPTATIEYVFSGQ (SEQ ID NO: 8)
MKTAVVLGISAWLASVTALSPAGWREQSIYQIVTDRFALTSGGSTPACTTDTQLGMFCNGTFQGIINKLDYI
QNMGFTAIWISPIVKNIDGGSPDGYTVDGSAYHGYWAQDIYQINPHFGTPQDLKQLSQSLHQRGMYLMVD
IVVNHNGYYCGLAEDGNCGPQGTIDYSIYNPENSEEYFHPFCEIDYSNATSILDCWEGDEIVPLPDLRTENTT
VQAMFDSWIKELVNNYTIDGLRIDSLQQSGSFFFPSFYSASGGLYMVGEVFQEYPAQVCPYQQAGMPGVL
NYPMFYYITDAFEDSAGSMSALANGISYMQGNCSDTTLLGSFLENQDNPRFPTTTGDIVRAMNAITFTLLQ
DGIPITYYGQEQHLNGSGVPENRQALWSSGGYDTTSQLYEMISKVNAIRTHAIEKASSFVTDKIQTPYYDNH
TIVTRKGDPGAQIVGVYSNLGENGDYYDLTLTKEETGFGPIEPVMEILTCTLLTTSFSGDLTVPMGGGQPRIL
FPYLGIIGSQICPGFTLKPPTGPNCSPAEITFEILANTTWGESVVVTGDIPSLGNWDVANGVHLNASEYTAAN
PLWVGTATGIDPQEVFQWKPVVIQTDGSYDWYDGDNIQDVSPGPGCNPTGTLQVTFY (SEQ ID NO: 9)
MRTSILFLAAQSTCALALSGADWRQQSIYQVVIDRFSLTNGDLTPKCTTDFELSNYCNGTFQGIISQLNYIQG
MGFTSIWISPIVKNIEGGSPNGYTKDGSPYHGYWAQDIYAINPHFGTEQDLKDLASELHDRGMFLMVDIVV
NHMGYYCGAAEEGSCGPEGSIDYSVFNPFDDESYFHSFCLIDYNNATSILDCWEGSQNVPLPDLRIEDVSVQ
KILDSWITDLVKNYSIDGLRIDSLQQSGSFFFPSFSNATGEIYMVGEVFQEYPAQVCPYQQPGMPGVMNYP
MYYYINDAFKSSSGNMKALSEGIAYMQGNCTDTTLLGSFLENQDNARFPSYTGDIVRAANAITFTLLQDGI
PITYYGQEQHLNGTGTPYNREALWSTGGYNTSSTLYKLISQVNAIRTKASELSSNFLTSKIKTPYTDDHTIVT
CKGEAGNQIIGVYSNLGENGAHYNLTLSKTVTGFNDSQVIVEVLSCTLSTTSPSGDLTLPMGAGEPKVLFPY
SVLQNSSICPNPSSPPTAPGQNCSSASITFNILVNTTYGESVVVAGNIPALGNWNVPSGLSLSADKYTAENPL
WSGTATGIDPQAAFQWKPVVVKTDGSYVYLPENNLMDVAPGPGCIPSGTIGYKWLSS (SEQ ID NO: 10)
MMLLTPILLGAASVVVALSPEQWRAQSIYQVVTDRFALDGGALSPTCTGDFELSIFCNGSFYGLIDKLQYIK
DMGFTALWVSPVVENIEGGTPDGYTKDGSPYHGYWAKDIYSINDHFGGSDGLKALSQALHNNDMYLMV
DVVVNHMGYYCGAALDGHCGPQGTVNYSIYNPENSEKYFHPFCEIDYNNATSILDCWEGSENVPLPDLRT
EDTVVQQMFDSWISGLVSDYSIDGLRIDSLQQSGSFFFPSFFNATGGMYMVGEVFQEYPKRVCSYQQEGMP
GVLNYPMFYYVTDAFSTSDGSMSGLANGISYMRGNCSDVTLLGSFLENQDNPRFPSLTEDITRSMNAITFV
MLQDGIPIVYYGQEQHLNGGEVPYNREALWSTGGYNQSAPLYEMITSINKARTAAIQQDASFVTDEMQIPY
YDDHVIVARKGRSGDQIVSIYDNRGSSGASSGLTLSQTDTGFSSAEQVMDILSCEQYTTASDGSLFMPLGGG
LPKALVTQSVLESSGLCTTYTADSVAGPTSVGSSTTCANATVVFDIYVTTSYGQSVVIAGDISALGDWTPAN
GLKLSANGYTADSPLWTGSAQVDPGTVFQWKPVIIETNESEDWYSGDNLQNASQAGCHPQSTTILLDWTS
(SEQ ID NO: 11)
AGLTPAEWRSQSIYQVVTDRFALDNGGNSPSCSGQSELNLYCNGTFAGIIDKLDYIQNMGFTAIWISPVVKN
IDGGSPNGYTPDGSAYHGYWAQDIYEINPHFGGASGLTDLSNALHSRGMYLMVDVVVNHMAYYCGTDG
GCGPGNSVNYGSFTPFNSESYFHPFCEIDYNNRTSILDCWEGDEIVPLVDLRTEDSDVQSIFNSWISNLIQTY
NIDGLRIDSLQQSGSFFFPGFNQAAGGMYMVGEVFNGSPSYVCPYQQAGMPGVLNYPMFFYITNAFQTSSG
SMSQLAQGISAMQSDCSDTTLLGSFLENQDNPRFPSQTSDLTRAQNAIAFTMLQDGIPITYYGQEQHLSGSG
VPLNREALWTSGGYDTSSPLYEMITTVNQLRTLAIKQNGGFVTYKIQVPYTDSNHIVTRKGNSGYQIVGVY
TNVGSAGASSTLSISSSETGFQASEPVMDVLSCTLYHTGTDGSLSFTMTGGLPRVFYNATALAESSLCTTYT
TASPPPGGCSAGTVVFDVYVQTQYGQSVVIAGNIPQLGNWSPANGLNLNANQYTASSPKWTGTITGVAPG
TTFQWKPIVVTNGNDNWYPGNNQQATTGSACSS (SEQ ID NO: 12)
MAPVRSLAGALLASLGLVAGLSPAEWRSQSIYQVVTDRFALDNGGNSPSCSGQSELNLYCNGTFAGIIDKL
DYIQNMGFTAIWISPVVKNIDGGSPNGYTPDGSAYHGYWAQDIYEINPHFGGASGLTDLSNALHSRGMYL
MVDVVVNHMAYYCGTDGGCGPGNSVNYGSFTPFNSESYFHPFCEIDYNNRTSILDCWEGDEIVPLVDLRT
EDSDVQSIFNSWISNLIQTYNIDGLRIDSLQQSGSFFFPGFNQAAGGMYMVGEVENGSPSYVCPYQQAGMP
GVLNYPMFFYITNAFQTSSGSMSQLAQGISAMQSDCSDTTLLGSFLENQDNPRFPSQTSDLTRAQNAIAFTM
LQDGIPITYYGQEQHLSGSGVPLNREALWTSGGYDTSSPLYEMITTVNQLRTLAIKQNGGFVTYKIQVPYTD
SNHIVTRKGNSGYQIVGVYTNVGSAGSSSTLSISSSETGFQASEPVMDVLSCTLYHTGTDGSLSFTMTGGLP
RVFYNATALAESSLCTTYTTASPPPGGCSAGTVVFDVYVQTQYGQSVVIAGNIPQLGNWSPANGLNLNAN
QYTASSPKWTGTITGVAPGTTFQWKPIVVTNGNDNWYPGNNQQATTGSACSSPAADIEFTWSS (SEQ ID
NO: 13)
MAPVRSLAGALLASLGLVAGLSPAEWRSQSIYQVVTDRFALDNGGNSPSCSGQSELNLYCNGTFAGIIDKL
DYIQNMGFTAIWISPVVKNIDGGSPNGYTPDGSAYHGYWAQDIYEINPHFGGASGLTDLSNALHNRGMYL
MVDVVVNHMAYYCGTNGGCGPGNSVNYGSFTPFNSESYFHPFCEIDYNNRTSILDCWEGDEIVPLVDLRT
EDSDVQSIFNSWISNLIQTYNIDGLRIDSLQQSGSFFFPGFNQAAGGMYMVGEVENGNPSYVCPYQQAGMP
GVLNYPMFFYITNAFQTSSGSMSQLAQGISAMQSDCSDTTLLGSFLENQDNPRFPSQTNDLTRAQNAIAFTM
LQDGIPITYYGQEQHLSGSGVPLNREALWTSGGYDSSSPLYKMITTVNQLRTLAIKQNGGFVTYKIQVPYTD
SNHIVTRKGNSGYQIVGVYTNVGSAGASSTLSLSSSETGFQASEPVMDVLSCTLYHTGSDGSLSFTMTGGLP
RVFYNATALAESSLCTTYTTATPPPGGCSAGTVVFDVYVQTQYGQSVVIAGNIPQLGNWSPANGLNLNAN
QYTASSPKWTGTITGVAPGTTFQWKPIVVTNGNDNWYTGSNQQATTGSACSSPATDIEFTWSS (SEQ ID
NO: 14)
LSAAEWRTQSIYFLLTDRFGRTDNSTTATCDTGDQIYCGGSWQGIINHLDYIQGMGFTAIWISPITEQLPQDT
ADGEAYHGYWQQKIYDVNSNFGTADDLKSLSDALHARGMYLMVDVVPNHMGYAGNGNDVDYSVFDPF
DSSSYFHPYCLITDWDNLTMVQDCWEGDTIVSLPDLNTTETAVRTIWYDWVADLVSNYSVDGLRIDSVLE
VEPDFFPGYQEAAGVYCVGEVDNGNPALDCPYQKVLDGVLNYPIYWQLLYAFESSSGSISDLYNMIKSVAS
DCSDPTLLGNFIENHDNPRFASYTSDYSQAKNVLSYIFLSDGIPIVYAGEEQHYSGGKVPYNREATWLSGYD
TSAELYTWIATTNAIRKLAISADSAYITYANDAFYTDSNTIAMRKGTSGSQVITVLSNKGSSGSSYTLTLSGS
GYTSGTKLIEAYTCTSVTVDSSGDIPVPMASGLPRVLLPASVVDSSSLCGGSGSNSSTTTTTTATSSSTATSKS
ASTSSTSTACTATSTSLAITFEELVTTTYGEEIYLSGSISQLGDWDTSDAVKMSADDYTSSNPEWSVTVTLPV
GTTFEYKFIKVESDGSVTWESDPNREYTVPECGSGETVVDTWR (SEQ ID NO: 48)
LSAAEWRTQSIYFLLTDRFGRTDNSTTATCNTGDQIYCGGSWQGIINHLDYIQGMGFTAIWISPITEQLPQDT
SDGEAYHGYWQQKIYDVNSNFGTADDLKSLSDALHARGMYLMIDVVPNHMGYAGSGNDVDYSVFDPFD
SSSYFHPYCLITDWDNLTMVQDCWEGDTIVSLPDLNTTETVVRTIWYDWVADLVSNYSVDGLRIDSVLEV
EPDFFPGYQEAAGVYCVGEVDNGNPALDCPYQDYLDGVLNYPIYWQLLYAFESSSGSISDLYNMIKSVASD
CSDPTLLGNFIENHDNPRFAYYTSDYSQAKNVLSYIFLSDGIPIVYAGEEQHYSGGDVPYNREATWLSGYDT
SAELYTWIATTNAIRKLAIAADSSYITYANDPIYTDSNTIAMRKGTSGSQVITVLSNKGSSGSSYTLTLSGSG
YTSGTKLIEAYTCTSVTVDSNGDIPVPMASGLPRVLLPASVVDDSSLCGGSGSSTSTTTSTATATTTSKTSTT
SSSSSSSSCTASATAIPITFEELVTTTYGEEIYLSGSISQLGDWDTSDAVKLSADDYTSSNPEWSVTVTLPVGT
TFEYKFIKVESGGSVTWESDPNREYTVPECGSGETVVDTWR (SEQ ID NO: 49)
LSAAEWRSQSIYFLLTDRFGRTDNSTTATCDTGEQIYCGGSWQGVINHLDYIQGMGFTAIWISPVTEQLPQD
TADGEAYHGYWQQEIYTVNSNFGTADDLKALSDALHARGMYLMVDVVPNHMGYAGDGDDVDYSVFDP
FDSSSYFHPYCLITDWSNLTMVRDCWEGDTIVSLPDLNTTETVVRTIWYDWVADLVANYSVDGLRIDSALE
VEPDFFPGYQEAAGVYCVGEVDNGDPTLDCPYQEYLDGVLNYPIYWQLLYAFESSSGSISDLYNMIKSVAS
DCSDPTLLGNFIENHDNPRFAYYTSDYSEAKNVLSYIFLSDGIPIVYAGEEQHYNGGDVPYNREATWLSGY
DTSAELYTWIATTNAIRKLAIEADSDYITYANDPFYTDSNTIAMRKGTSSQQVITVLSNKGSSGSSYTLTLSG
SGYTSGTELIEAYTCTSVTVDSNGDIPVPMDSGLPRVFLPASSINSSSSSLCGGSSTTTTTTTSTSPSTATSSST
ATATTTCTTLPITFIESVTTTYGEEIYLSGSITQLGDWDTSDAVLLSADNYTSSNPEWYVEVSLPTGTSFEYKF
IKKESDGSVTWESDPNREYTVPEECGGVTVDDSWR (SEQ ID NO: 50)
LTPAEWRSQSIYFLLTDRFGREDNSTTASCDLSERIYCGGSWQGIINHLDYIQGMGFTAIWITPVTEQLPQDT
GEGEAYHGYWQQEIYSVNSNYGTAADLLALSEALHDRGMYLMVDVVANHMGYAGAGDSVDYSVFSPFD
SSSYFHSYCLISDYSNQENVEDCWLGDTTVSLPDLNTDLTSVQTLWYDWVADLVSNYSIDGLRIDTVKHV
QESFWPGYNSAAGVYCVGEVFDGDPAYTCPYQNYLDAVLNYPIYYQLLYAFESSSGSISGLYDMISSVASD
CADPTLLGNFIENHDNPRFASYTDDYSQAKNVISFVFLSDGIPIIYAGQEQHYSGGSDPENREATWLSGYSTT
AELYQFIATTNKIRALAVSSDSSYITTKNDPFYTDSNTIAMQKGSSGSQVVTVLSNMGASGSSYTLTLSGSG
YASGTELMEMYTCTSVTVDSSGNIAVPMASGLPRVFMLASSAGSLCGSSTTTTTSKITSTTSTSSTSSTSTSC
TQTTDVPVLFKELVTTTYGEDIYISGSISELGDWDTANAVALSSSGYTSSNPLWQVVVSIPAGTSFEYKFFET
GSSSSVTWESDPNRSYTVPTACSGSTATVTATWR (SEQ ID NO: 51)
LTPAEWRSQSIYFLLTDRFGRTDNSTTATCNVSDRIYCGGSWQGIINHLDYIQGMGFTAIWITPVTEQLSQDT
GDGEAYHGYWQQEIYNVNTNYGTAADLLALSKALHSRGMYLMVDVVANHMGYDGAGNTVDYSVENPF
DSSSYFHSYCEITDYSNQTNVEDCWLGDTTVSLPDLDTTLSSVQTIWYNWVTGLVSNYSIDGLRIDTVKHV
QKSFWPGYNSAAGVYCMGEVFDGDPAYTCAYQSYLDGILNYPVYYQLLYAFESTSGSISNLYNMINSVAS
DCSDPTLLGNFIENHDNPRFASYTSDYSQAKNVISFIFFSDGIPIVYAGQEQHYSGGSDPANREATWLSGYD
KTAQLYTYITTTNKIRALAISKDSAYITSKNTAFYSDSNTIAMKKGSSGSQVITVLSNRGSSGSSYTLSLSGSG
YSSGKKLMEMYTCTAVTVDSSGNIAVPMASGLPRVYMLASSACSICSSTCSTTTSTASSTSSSTTTASTTLKT
TASSASTSCTQATALPVLFKEIVTTSYGQNIYISGSISQLGSWDTSSAVALSADQYTSSNHLWYVVVTIPVGT
SFQYKFIEETSGYSTITWESDPNRSYTVPTGCVGSTATVTATWR (SEQ ID NO: 52)
LTPAEWRSQSIYFLLTDRFGRQDNSTTAACDVTQRLDYIQGMGFTAVWITPVTEQFYEDTGDGTSYHGYW
QQNIYEVNSNYGTAQDLKSLANALHARGMYLMVDVVANHMGYDGAGTSVDYSVFNPFNSSSYFHPYCLI
SDYSNQTNVEDCWLGDTTVSLPDLNTTETTVRTIWYDWVKALVANYSIDGLRIDTVKHVEKAFWPDYNS
AAGVYCVGEVFSGDPAYTCPYQNYLDGVLNYPIYYQLLYAFESTSGSISNLYNMISSVASDCADPTLLGNFI
ENHDNPRFASYTSDYSQAKNVVSFIFFSDGIPIVYAGQEQHYSGGADPANREAVWLSGYSTSATLYSWIAST
NKIRKLAISKDSAYVTSKNNPFYYDPNTLAMRKGSTAGAQVITVLSNKGSSGSSYTLSLSGTGYSAGATLV
EMYTCTTLTVDSSGNLAVPMASGLPRVLVPASWVSGSGLCGGSVSTTATTTTSATTTTTATTTTACTSATA
LPILFEELVTTTYGENIYLTGSISQLSNWDTSSAIALSAGNYTSSNPEWYVTVTLPIGTSFQYKFFKKESDGSI
TWESDPNRSYTVPTGCAGTTVTVSDTWR (SEQ ID NO: 53)
LTPAEWRTQSIYFLLTDRFGREDNSTTAACDVTERLDYIQGMGFTAIWINPVTEQFYEDTGDGTSYHGYWQ
QNIYEANHNYGTAQDLKSLADALHARGMYLMVDVVANHMGYDGAGNSIDYSVFTPFDSSSYFHPYCLIS
DYSNQTNVEDCWLGDTTVSLPDLDTTDAKVRTIWYDWVKGLVANYSIDGLRIDTVKHVEKDFWPGYND
AAGVYCVGEVFSGDPTYTCPYQNYLDGVLNYPIYYELLYAFESPRGSISNLYNMISSVASDCADPTLLGNFI
ENHDNPRFASYTSDYSQAKNVISFMFFSDGIPIVYAGQEQHYSGGADPANREAVWLSGYSTSATLYSWIAS
TNRIRKLAISKDTAYITSKNNPFYYDSNTLAMRKGSVAGAQVVTVLSNKGSSGSSYTLSLSGTGYSAGASL
VEMYTCTTLTVDSSGNLAVPMASGLPRVLVPSSWVSGSGLCGSGSTTTTTTTTTATATTTACTSATALPILF
EEILTTTFGENIYLSGSISQLGNWDTSSAIALSASKYTSSNPEWYVTVTVPVGTSFQYKFFKNVSDGSIVWET
DPNRSYTVPSGCGGTTVTLSGTWR (SEQ ID NO: 54)
LTPAEWRSQSIYFLLTDRFGREDNSTTAACDVTQRLDYIQGMGFTAIWITPVTEQFYEDTGDGTSYHGYWQ
QNIYEVNSNYGTAQDLKSLADALHARGMYLMVDVVANHMGYDGAGNSVDYSVFTPFGSSSYFHPYCLIS
DYSNQTNVEDCWLGDTTVSLPDLDTTDTTVQTICTRTWAWMILTVIVDGLRIDTVKHVEKDFWPGYNDA
AGVYCVGEVFSGDPTYTCPYQNYLDGVLNYPIYYQLLYAFESTSGRISNLYNMLSSVASDCADPTLLGNFIE
NHDNPRFASYTSDYSQAKNVISFMFFSDGIPIVYAGQEQHYSGGADPANREAVWLSGYSTSATLYSWIAST
NKIRKLAISKDSAYITSKNNPFYYDSNTLAMRKGSTAGSQVITVLSNKGFSGSSYTLSLSGTGYSAGTTLVE
MYTCTSLTVDLSGNLAVPMASGLPLVLVPSSWVSGSGLCGSSVSTTATATSTTKTTIATTTAACTSATALPI
LFEELVTTTYGENIYLTGSISQLGNWDTSSAIALSASKYTSSNPEWYVTVTLPVGTSLQYKFFKKESDGSIV
WESDPNRSYTVPTGCAGTTVTASDTWR (SEQ ID NO: 55)
LSPAEWRSQSIYFLLTDRFGRTDNSTTAECDVEKRLYCGGTWQGIINQLDYIQGMGFTAIWITPVTEQLSQD
TGYGEAYHGYWQQKIYNLDSHLGTADDLKALSKALHDRGMYLMVDVVANHMGYDGPGNSVDYSVFDP
FDSSSYFHKYCLISNYDDQNDVQNCWLGDTTVSLPDLNTKDAEVRELWYDWVTGLVSNYSIDGLRIDTVK
HVEKTFWEGYNQAAGVYCVGEVFDGDPKYTCAYQSYLDAVLNYPIYYPLLNAFKAPGGNMGDLYNMVN
SVASHCSDPTVLGNFIENHDNPRFASYTDDYSLAKNAITFMFLSDGIPIIYAGEEQHYSGGSDPGNREAVWL
SGYSTDAELYKFIATTNKIRSLVILKDNKYVTSKNVPFYQDDNTFAMRKGSGNSQAITVLSNAGSSSDSYSV
TVKGAEYSSGTQLIDLYSCAKVTVGDDGSISLQVESGLPKVLVPASYAGDALCGGSSAPSIA (SEQ ID
NO: 56)
LSPAEWRSQSIYFLLTDRFGREDNSTTASCDVSNRLDYIQDMGFTAIWITPVTEQLQGDTGYGEAYHGYWQ
QDIYDVNENYGTAEDLLALSNALHDRGMYLMVDVVANHMGYNGAGNTVDYSVFNPFDSSSYFHSYCLIT
NYDNQDNVEDCWLGDTTVSLPDLNTDLSSVQTIWYDWISDLVSNYSIDGLRIDTVKHVQKSFWPSYNSAA
GVYCVGEIFDGNPAYTCDYQNYMDGVLNYPIYYQLLYAFQSTSGSISDLYNMIGTVASDCKDPTLLGNFIE
NHDLPRFASFTSDYSLAKNVLSFLFFSDGIPIVYAGQEQHYSGKDDPANREATWLSGYSTSAELYKHISTTN
KIRKLAASSDSSYITSRVIIVSSKLDKPKGLTNSMQNTPFYQDSHTIAMKKGSSGSQVITVLSNYGSSGSSYTL
TLGSSGYSSGTELMEMYTCTTATVDSSGNIPVPMVSGLPRVLMLASVAKSSGPCASSGSPTTSTASSSSTTM
SACTKATALPVLFKETVSTSYGEDIYISGSISRLGSWDASNAVALSADQYTSSNPLWYVSITLPVGTSFEYKF
IKKTSGSSTVTWESDPNRSYTVPTACSGSTATVTATWR (SEQ ID NO: 57)
ATPAEWRSQSIYFLLTDRFARTDGSTTASCNTEDRKYCGGTWQGIIDKLDYIQGMGFTAIWITPVTGQLPQH
TAYGDAYHGYWQQDIYALNSNYGTADDLKALASALHDRGMYLMVDVVANHMGYDGAGDSVDYSVFK
PFNSKDYFHNFCLIQNYDDQTQSENCWLGDNSVSLPDLDTTREDVKGLWYDWVEALVSNYSIDGLRIDTV
KHVQKDFWPGYNQAAGVYCIGEVLNGDPAYTCPYQNVMDGVLNYPMYYPLLNAFKSTSGSISDLYNMIN
TVKSTCPDSTLLGTFVENHDNPRFASYTNDMSLAKNVAAFIILADGLPIIYAGQEQHYAGGNDPANREATW
FSGYSTTSEIYKLIASSNAIRNHAISTDSGYLTYKNWPVYQDASTIAMRKGTDGSQIITVLSNQGSSGGSYTL
SLTGTGYTAGQQLTEITTCSTVTVGLDGKVPLPMEKGLPKVLYPSAKLSGSKVCSSS (SEQ ID NO: 58)
ATPADWRSQSIYFLLTDRFARSDGSTTASCNTEDRKYCGGTWKGIIDQLDYIQGMGFTAIWITPVTGQLPQD
TGYGEAYHGYWQQDIYSLNDKYGTADDLKALADALHNRHMYLMVDVVANHMGYSGAGDSVDYSVFKP
FNSKDYFHPYCPIKDYNDQTQAENCWLGDNTVSLPDLDTTKSEVKDLWYKWVGDLVSNYSIDGLRIDTVK
HVQKDFWPGYNKAAGVYCVGEVLNGDTAFTCAYQSVLDGVLNYPMYYPLLNAFKSTSGNIGDLYNMIN
NVKSNCPDSTLLGTFIENHDNPRFASYTNDMSLAKNVAVFTILADGIPIIYAGQEQHYAGGEDPANREATW
LSGYSTKSELYTLIASSNAIRNHAIAQDPKYVTYKNYPIYQDETTIAMRKGSDGSQVVTVLSNKGSSGSSYT
LSLGDTGYSSGQNVTEIYTCETLTVGSDGKVSVPMNNGLPRVLYPSEKLKGSSLCSE (SEQ ID NO: 59)
ATPADWRSQSIYFLLTDRFARTDGSTSATCNTEDRKYCGGTWKGIIDQLDYIQGMGFTAIWITPVTSQLTQD
TAYGEAYHGYWQQDIYSLDSHLGTADDLRALASALHERGMYLMVDVVANHMGYDGAGDSVDYSVFNP
FNSKDYFHPFCLIQDYNDQTQSENCWLGDNSVSLPDLDTTTQDVKNIWYDWVEELVSNYSIDGLRVDTVK
HVQKDFWPDYNKAAGVYCIGEVLNGDPSYTCPYQEVMDGVLNYPIYYPLLNAFKSTSGNMNDLYNMINT
VKSDCPDSTLLGTFIENHDSPRFASYTNDMALAKNVAAFIILADGIPIIYAGQEQHYAGGNDPSNREATWLS
GYSTTSDLYKLIASSNAIRNHAVSTDKGYVTYKNWPIYKDTTTIAMRKGTDGSQIITILSNKGASGDGYTLS
LDGTGYDAGTELTEILSCSTLTVGSDGKVPVPMEKGLPRVLYPSAKLGDSKICT (SEQ ID NO: 60)
ATPEQWRSQSIYFLLTDRFARTDGSTTASCDTTARKYCGGTWQGIIKQLDYIQGMGFTAIWITPVTEQLPQD
TSEGTAYHGYWQQDIYSVNSNYGTADDLKALGSALHDRGMYLMVDVVANHMGYAGAGNSVDYSLFSPF
NSQTYFHPLCFISNYDNQTNVENCWLGDNVVPLADLDTTKSDVQKIWYNWVGSLVSNYSIDGLRIDTVKH
VQKDFWPGFNDAAGVYCIGEVFDGDPAYTCPYQDVLDGVLNYPTYYPLLKAFQSTSGSMSDLYNMINTV
KSQCADSTLLGTFVENHDTPGSHRKYTNDMALAKSAAAFIILSDGIPIIYAGQEQHYSGGTDPANREAVWL
SGYSKTSELYKLIASANAIRNHAISKDPEYVTYKNNPIYKDTSTIAMRKGADGAQVITVLSNLGASGSSYTL
SLSGTGYDVGQQLTEVFSCATVTVDSDGKVPVAMASGLPRAFYPTAGLNGSTICA (SEQ ID NO: 61)
ATPADWRSQSIYFLLTDRFARTDGSTTAACNTEDRKYCGGTWQGIIDKLDYIQGMGFTAIWITPVTGQLPQ
NTAYGEAYHGYWQQDIYSLNENYGTPDDLKALASALHTRGMYLMVDVVANHMGYDGAGASVDYSVFK
PFNSQEYFHPFCLIQNYNDQTQSEDCWLGDNSVSLPDLDTTKDEVKNEWYEWVGSLVSNYSIDGLRVDTV
KHVQKDFWPGYNKAAGVYCIGEVLNGDPAYTCPYQDVMDGVLNYPIYYPLLNAFKSTSGSMDDLYNMIN
TVKSDCPDSTLLGTFVENHDNPRFASYTNDIALAKNVAAFIILNDGIPIIY AGQEQHFAGGNDPANREATWL
SGFSTDSEIYKLIASANAIRSHAISIDTGFVTYKNWPIYKDTTTIAMRKGTDGSQVVTILSNKGASGDSYTLSL
GNTGYTAGQKLTEVIGCTTVTVGSDGTVPVPMAGGLPRILYPTEKLGDSKICSSA (SEQ ID NO: 62)
ATPAEWRSQSIYFLLTDRFARTDGSTTASCNTEDRKYCGGTWQGIIDKLDYIQGMGFTAIWITPVTGQLPQH
TAYGDAYHGYWQQDIYTLNRNYGTADDLKALASALHDRGMYLMIDVVVNHMGYDGAGDSVNYSVFKP
FNTKDYFHNFCFIQNFDDQTQSENCWLGDNRVSLPDLDTTREDVKGLWYDWVEAFVSNYSIDGLRVDTV
KHVQKDFWRGYNEAAGVYCIGEVENGDPAYTCPYQNIMDGVLNYPMYYPLLNAFKSTSGSISNLYNMINT
VKSTCPDSTLLGTFVENHDNPRFASYTNDMSLAKNVAAFIILADGLPIIYAGQEQHYAGGNDPANRDATWF
SGYSTTSELYKLIASLNAIRRHAINTDSGYLSYKNWPIYQDASTIAMRKGTDGSQIITVLSNQGSSGGSYTLS
LTGTGYTAGEQLHEIINCSTVTVGSDGKVLLPMEKGLPRVLYPSAKLNGSKVCSSS (SEQ ID NO: 63)
LNAAEWRSQCIYFLMTDRFARTDGSTTAPCDVGQRGYCGGSWKGIIDHLAYIQGMGFTAIWITPIVEQIPQT
TTEGTGFHGYWPQNIYSVNSHFGTADDIRALSKALHDRGMYLMMDGVANHMGFNGPGASNGFSTFTPFN
SASYFHSYCPINNYNDQWQVENCYLRDSVVSLTDLNTQSSAVRGIWYDWVKDLVANYSVDGLRIDTAKH
VEKDFWSGYTQAAGVYSVGEILHGDPAYTCPYQGYMDGVMNYPIYYQLVNAFKSSSGSISSLSNMISSVAS
KCKDPTLLGNFIENHDNPRFPSYTSDISQAKSVIAYVFLTDGIPIIYSGQEQHLSGGADPYNREALWLSGYST
NSELYKFIAMTNKIRRLAISKDLNYLPARNNPFYTDSNTIAMKKGSGGSNVITVLTNSGSNAGSYTLNLNSH
GYSSGSSLIELYTCSSVQVDSNGNLPVPMSSGLPRVLVPSAWVPGSGLCGASSTTSLATPTVTSPSPGTCAPS
TAIPVIFKERVATSYGENIFLSGSIDQLGNWDTSKAVALSASGYTSSNPVWSVKLDLHAGTYFQYKFIKKGQ
DGSVMWESDPNRSYTLPSGCVETAISIADSWR (SEQ ID NO: 64)
ATPADWRSQSIYFLLTDRFARTDNSTTASCNTGARQYCGGTWQGIINQLDYIQGMGFTAIWITPVTGQIPQE
TGYGEAYHGYWQQDAYALNSHYGTADDLKKLVSALHARGMYLMVDVVANHMAYNGSADTTDYSAYN
PFNSKEYFHKICSIEDNNNQTQTEDCWLADSIVSLPDLNTTQTDVKNMWYDWIGSLVSNYSIDGLRIDTVK
NVQQNFWPDYNTAAGVYCVGEVFDGDAEYTCPYQNYLDGVLNYPMYYPILRAFQSTSGSISDLYNMINT
VKSTCKDSTLLGTFVENHDNPRFANYTSDMSLAKNAAAFTMLADGIPIIYAGQEQHYHGGNDPYNREATW
LAKYSTTSPLYELIATSNRIRSHAISQDSGYITYKNYPIYQDNSTLAMRKGYNGSQTITVLSNLGASGKTYNL
SLPGTGYTANQQLMEIYTCTNITVDSSGSVSVPMASGLPRVLYPADKLNGSLLCNSTTSSATSLSPLSNSAIPI
YLTLAAFVVQLSQL (SEQ ID NO: 65)
LSPAEWRGQSIYFLLTDRFARADNSTTAPCDVTDRVYCGGSWQGIINQLDYIQGMGFTAIWVSPVTEQLPQ
DTEYGEAYHGYWQQDIYNLNKHFGTPDDLNALADALHERGMYLMVDVVPNHMGYDGPGDSVDYGVFE
PFSSSSYFHPFCFIENYDNQDNVEQCWLGDSKVPLPDLDTSREDVQNIWYDWVSGLVSNYSVDGLRLDTA
KHVQKNFWPDYNKAAGVYCVGEVLSGDPKYTCPYQEYMDAVLNYPIYYQLLDAFKSTNGPTDKVYNMI
SRVASSCRDPTLLGNFIENHDAPRFASYTQDLALAKNAIAFLFLTDGIPIVYAGQEQHYSGGDDPFNRGAIW
LSGYSTEAALYRFIGDTNKVRSQAIAMDSAYLTSQNEPFYHDDSTIAMRKGPKGSEVLTVLTNSGSKGSYT
VSIDAGYGSGTELVDLYSCNTATVGSDGTVSVKISAGMPQALVPASAAGNSALCGNSSK (SEQ ID NO: 66)
LTAAEWRSQSIYFLLTDRFALTSNSTTASCDVADGLYCGGSWQGVINHLDYIQGMGFTAIWITPVTENFEG
DTSDGEAYHGYWQQNAYATNSHYGASSDLLKLSEALHARGMYLMVDIVVNNMAYDGAGTSVDYSIFNP
FPSKSYYHSYCLIDYSTYNATDWNVCWEGDNIVSLPDIDTTQTYVKDTWGTWVESFVANYSIDGLRIDSAG
HIQQDFFTAFEESSGVYCIGEVDYGDPAVVCPYQNYLSGVLNYPMYYQLLYAFESSSGSISNLYNMINTVKS
DCADTSLLGNFIENHDNPRFAYYTSDYSEAKNVISFIFLTDGIPILYYGQEQHYNGGNIPLNREALWTSDYST
TAQLYTHTKTSNAIRSLAITKDSAFLTYKNTPIYQDSNTIAMRKGTTGLQLVTVLSNLGASGSSSTLTLSGSG
YTSGTVVTELYTCTNVTVSSSGTIAVPMASGSPRAFLPWSSVSGSSLCSGSGSSCTAASTVAVTFEEVVTTT
YGQEVYISGSISQLGNWSTSSAVLLSASQYTSSDPVWTVTISLPAGESFQYKFIIVNTDGSITWESDPNRSYT
VPTGCAGLTATVDDTWR (SEQ ID NO: 67)
ATPDDWRSQSIYFLLTDRFARADNSTTATCNTEDGIYCGGSWQGVINQLDYIQGMGFTAIWISPVTGQLTE
VTSDGSSYHGYWQQDIYALNENFGTSDDLNDLASALHDRGMYLMVDVVANHMGYAGDPTTVDYSVFNP
FNSQDYFHPYCALTDYTNQTMTEECWMGDDTVCLPDLDTESTSVQDIWFSWIGDLVANYSIDGIRIDTVAE
VSKDFWATYNEKAGVYAVGEVDNGDVTYACPYQTALDGILNYPTYFPLVRAFESSSGSISELADMINSVKS
DCTDTNLLGSFSENHDNPRFASYTQDLALAKNVLTYTILADGIPIIYAGQEQHYDGAEDPANREATWLSGY
NTTSELYTWVAKMNKIRSFAIKSDDGYTTYNNYPIYQDDNNLGMRKGSNGSQIITVVSNLGADGGQTTLT
VSGGGYTAGTVLTELITCTSVTVGDGGDISVSLDSGLPSVLYPASKLNGTGSPC (SEQ ID NO: 68)
ATPAQWRSQSVYFLLTDRFARTDGSTTAACDTDARAYCGGTWQGIIDHLDYIQGMGFTAIWITPVTENLPQ
DTGDGTSYHGYWQQDVYSLNSNYGTPDDLRALSSALHDRGMYLMVDVVANHMGYAGPGSSVDYTVFTP
FNDQKYFHPYCSISNYDDQSNVEDCWLGDSTVSLPDLDTTRSDVQDMWYSWVKGLVANYSVDGLRIDTV
KHVQKDFWPGYNDAAGVYCVGEVFDGDPSSTCDYQNYLDGVLNYPMYYPLLRAFSSTSGSISDLYNMIN
TVKSECADSTLLGTFVENHDNPRFASYTSDISLAKNALAFTILSDGIPIIYAGQEQHYSGGNDPANREAVWL
SGYSTTSELYKFIAVSNQIRNHAISVDGDDYLTYKTYPIYQDTTTLAVRKGSLITVLSNLGSSGSSYTLSLGG
TGYSSGQELMEIYSCTTVTADSSGNIAVPMGSGLPKAFYPTANLGGSGICGK (SEQ ID NO: 69)
ATPAQWRSQSIYFLLTDRFARTDNSTTAECDTSAGRYCGGSWQGIINQLDYIQGMGFTAIWITPVTAQVEDS
SSGDAYHGYWQQDLYFLNSQLGTKDDLLALSDALHERDMYLMVDVVANHMGYDGAADSVDYSVFNPF
NSQDYFHSPCSIDDYDDQTQVEECWLSTSAVSLPDVDTTRDDVKTLWYDWVEALVSNYSIDGLRVDTVRH
VQKDFWADFNDAAGVYCVGEVLQGDPEYTCAYQELMDGVLNYPIYYPLLRAFSSTSGSISELYDMINSVK
STCADSTLLGSFIENHDNPRFASETDDISLAKNVAAFVILSDGIPIIYAGQEQHYAGGEDPENREATWLSGFD
TSSELYKLIAASNAIRTHAIGQDDEWITYKNYPIYQDTSSLAMRKGNNGTQVVTVLTNAGAGGNSYTLSLP
DTGYSAGAALTEVLSCTDITVSDNGEVPVPMESGLPRVLYPTAKLEGSGICQ (SEQ ID NO: 70)
ATPAEWRSQSIYFLLTDRFARTDNSTTAECDTSAKYCGGTWQGIINQLDYIQGMGFTAIWITPVTANLEDG
QHGEAYHGYWQQDIYALNPHFGTQDDLRALSDALHDRGMYLMVDVVANHFGYDAPAASVDYSAFNPEN
SADYFHTPCDITDYDNQTQVEDCWLYTDAVSLPDVDTTNEEVKEIWYDWVGDLVSDYSIDGLRIDTARHV
QKDFWRDYNDAAGVYCVGEVFQGDPDYTCGYQEVMDGVLNYPIYYPLLRAFSSTSGSLSDLANMIETVK
YTCSDATLLGNFIENHDNPRFASYTDDISLAKNVAAFVILSDGIPIIYAGQEQHYSGAGDPANREATWLSGY
DSTSELYQFISKTNQIRNHAIWQNETYLSYKNYAIYNENNVLAMRKGFDGSQIITILTNAGADAGSSTVSVP
NTGFTAGAAVTEIYTCEDITVSGSGEVSVPMESGLPRVLYPKAKLEGSGICGL (SEQ ID NO: 71)
ATPDDWRSRSIYFLLTDRFARTDNSTTATCETLYGVCSKTTVEDHGRLDYIQDMGFTAIWISPVTEQLPQST
ADGEAYHGYWQQDIYALNSNFGTAEDLQNLATALHDRGMYLMVDVVANHFGYAGAGDDVDYAIFNPF
NSESYFHPFCLITDYSNETMVEECWEGDNIVSLPDLDTESTAVQNICLTRSYDNEDEATANGNYEPVDGLRL
DSVMEVQKDFWPDWNSASGVYCVGEVDDGDPTFTCPYQKYLDGVLNYPTYFPLVRAFESSSGSISELYDM
INEVKSDCVDSNLLGSFSENHDNPRFASYTSDFSLAKNALAFTILSDGIPIIYAGQEQHYDGGNNPYNREAT
WLSGYDTSAELYTFIAVLNQIRNYAIQWDNGYTTYKTYPIYQDDNNLAIRKGSDESQIITVLSNVGTDADSY
SLTISGTGYTAGEVLTDLISCTNVTVDDSGDVSVTMTGGLPSVLYPTYKLVYDGHPC (SEQ ID NO: 72)
ATPAEWRSRSIYFMLTDRFARTDGSTTAACNTGDRKYCGGTWQGIIDKLDYIQGMGFTAIWITPVTSQLTV
DTPYGEPYHGYWQQDIYALNSNYGTADDLKALAAALHERDMYLMVDVVANHMGYNGAGADVDYTKF
NPFNDAKYFHPYCPITDYDNDTMAQNCWLGDDKVSLPDLDTQSMEVQDIWYDWVRSLVSNYSKTDEYK
VDGLRIDTVKHVQKDFWPGYNKAAGVYCVGEVFDGDVDYTCPYQEVMDGVLNYPVYYPLLKAFQSTSG
SMADLFNMINRVKSTCKDSTLLGNFLENHDNPRFASVTDDIALAKNAATFTIMADGIPIVYAGQEQHYSGG
EDPANREALWLSGFNTDSELYKLIATVNGARNQAIAKSTNYTIYQNYPIYKDSSTIAMRKGYDSGQTITVLT
NLGAGGKDYSVSIPDTGFAAGAKLTEVISCVSVTAGESGEVSVPMAGGAPRILLPTSLLEGSTLCSS (SEQ
ID NO: 73)

What is claimed is:

1. A method for increasing starch digestibility and glucose yield in in the small intestine of a ruminant animal comprising adding at least one glucoamylase (EC 3.2.1.3) enzyme and at least one acid stable alpha-amylase (AsAA) enzyme as a feed additive to feed for the ruminant, wherein said at least one glucoamylase and at least one AsAA has at least about 20% activity at pH less than or equal to about 3 in at least one of three digestive chambers of a ruminant comprising a rumen, an abomasum and/or a small intestine.

2. The method of claim 1, wherein said at least one glucoamylase and at least one AsAA are capable of hydrolyzing raw starch under conditions comparable to those found in the rumen or abomasum.

3. The method of claim 1 or claim 2, wherein the at least one AsAA is a member of glycoside hydrolase family 13 (GH 13) family or is a member of EC 3.2.1.1 or a variant or functional fragment thereof.

4. The method of any one of claims 1-3, wherein the at least one AsAA is at least about 60% identical to the amino acid sequence of SEQ ID NO: 3 or SEQ ID NO:4 or a variant or functional fragment thereof.

5. The method of any one of claims 1-3, wherein the at least one AsAA is at least about 60% identical to the amino acid sequence of SEQ ID NO:6 or a variant or functional fragment thereof.

6. The method of claim 4, wherein the at least one AsAA comprises at least one of SEQ ID NOs: 8-21 or a variant or functional fragment thereof.

7. The method of claim 5, wherein the at least one AsAA comprises at least one of SEQ ID NOs: 22-73 or a variant or functional fragment thereof.

8. The method of any one of claims 1-7, wherein the at least one glucoamylase is at least about 60% identical to the glucoamylase of SEQ ID NO:5 or SEQ ID NO:7, or a variant or functional fragment thereof.

9. The method of any one of claims 1-8, wherein the ratio of glucoamylase to AsAA is about 70:30 to 96:4.

10. The method of claim 9, wherein the ratio of glucoamylase to AsAA is about 96:4.

11. The method of any one of claims 1-10, wherein the at least one glucoamylase and/or at least one AsAA has at least about 20% activity at pH less than or equal to about 2.5.

12. The method of any one of claims 1-10, wherein the at least one glucoamylase and/or at least one AsAA has at least about 20% activity at pH less than or equal to about 3 for at least about 60 minutes.

13. The method of any one of claims 1-12, further comprising adding at least one hemicellulase as a feed additive to the feed.

14. The method of any one of claims 1-13, further comprising adding betaine as a feed additive to the feed.

15. The method of any one of claims 1-14, further comprising adding at least one essential oil as a food additive to the feed.

16. The method of claim 15, wherein the essential oil comprises cinnamaldehyde and/or thymol.

17. The method of any one of claims 1-16, further comprising adding at least one direct fed microbial (DFM) as a feed additive to the feed.

18. The method of claim 17, wherein the direct fed microbial is a Megasphaera sp., Bacillus sp., a Propionibacterium sp., and/or an Enterococcus sp.

19. The method of any one of claims 1-18, wherein the ruminant is a beef cow, dairy cow, goat, sheep, giraffe, yak, deer, elk, antelope, water buffalo, or buffalo.

20. A method for increasing milk production in a ruminant animal comprising adding at least one glucoamylase (EC 3.2.1.3) enzyme and at least one acid stable alpha-amylase (AsAA) enzyme as a feed additive to feed for the ruminant.

21. The method of claim 20, wherein the at least one AsAA is a member of glycoside hydrolase family 13 (GH 13) family or is a member of EC 3.2.1.1 or a variant or functional fragment thereof.

22. The method of claim 20 or claim 21, wherein the at least one AsAA is at least about 60% identical to the amino acid sequence of SEQ ID NO: 3 or SEQ ID NO:4 or a variant or functional fragment thereof.

23. The method of claim 20 or claim 21, wherein the at least one AsAA is at least about 60% identical to the amino acid sequence of SEQ ID NO:6 or a variant or functional fragment thereof.

24. The method of claim 22, wherein the at least one AsAA comprises at least one of SEQ ID NOs: 8-21 or a variant or functional fragment thereof.

25. The method of claim 23, wherein the at least one AsAA comprises at least one of SEQ ID NOs: 22-73 or a variant or functional fragment thereof.

26. The method of any one of claims 20-25, wherein the at least one glucoamylase is at least about 60% identical to the glucoamylase of SEQ ID NO:5 or SEQ ID NO:7, or a variant or functional fragment thereof.

27. The method of any one of claims 20-26, wherein the ratio of glucoamylase to AsAA is about 70:30 to 96:4.

28. The method of claim 27, wherein the ratio of glucoamylase to AsAA is about 96:4.

29. The method of any one of claims 20-28, wherein the at least one glucoamylase and/or at least one AsAA has at least about 20% activity at pH less than or equal to about 2.5.

30. The method of any one of claims 20-29, wherein the at least one glucoamylase and/or at least one AsAA has at least about 20% activity at pH less than or equal to about 3 for at least about 60 minutes.

31. The method of any one of claims 20-30, further comprising adding at least one hemicellulase as a feed additive to the feed.

32. The method of any one of claims 20-31, further comprising adding betaine as a feed additive to the feed.

33. The method of any one of claims 20-32, further comprising adding at least one essential oil as a feed additive to the feed.

34. The method of claim 33, wherein the essential oil comprises cinnamaldehyde and/or thymol.

35. The method of any one of claims 20-34, further comprising adding at least one direct fed microbial (DFM) as a feed additive to the feed.

36. The method of claim 35, wherein the direct fed microbial is a Megasphaera sp., Bacillus sp., a Propionibacterium sp., and/or an Enterococcus sp.

37. The method of any one of claims 20-36, wherein the ruminant is a beef cow, dairy cow, goat, sheep, giraffe, yak, deer, elk, antelope, water buffalo, or buffalo.

38. A feed additive composition comprising at least one glucoamylase (EC 3.2.1.3) enzyme and at least one acid stable alpha-amylase (AsAA) enzyme, wherein said at least one glucoamylase and at least one AsAA has at least about 20% activity at pH less than or equal to about 3 in at least one of three digestive chambers of a ruminant comprising a rumen, an abomasum and/or a small intestine.

39. The feed additive composition of claim 38, wherein said at least one glucoamylase and at least one AsAA are capable of hydrolyzing raw starch under conditions comparable to those found in the rumen or abomasum.

40. The feed additive composition of claim 38 or claim 39, wherein the at least one AsAA is a member of glycoside hydrolase family 13 (GH 13) family or is a member of EC 3.2.1.1 or a variant or functional fragment thereof.

41. The feed additive composition of any one of claims 38-40, wherein the at least one AsAA is at least about 60% identical to the amino acid sequence of SEQ ID NO: 3 or SEQ ID NO:4 or a variant or functional fragment thereof.

42. The feed additive composition of any one of claims 38-40, wherein the at least one AsAA is at least about 60% identical to the amino acid sequence of SEQ ID NO:6 or a variant or functional fragment thereof.

43. The feed additive composition of claim 41, wherein the at least one AsAA comprises at least one of SEQ ID NOs: 8-21 or a variant or functional fragment thereof.

44. The feed additive composition of claim 42, wherein the at least one AsAA comprises at least one of SEQ ID NOs: 22-73 or a variant or functional fragment thereof.

45. The method of any one of claims 38-44, wherein the at least one glucoamylase is at least about 60% identical to the glucoamylase of SEQ ID NO:5 or SEQ ID NO:7, or a variant or functional fragment thereof.

46. The feed additive composition of any one of claims 38-45, wherein the ratio of glucoamylase to AsAA is about 70:30 to 96:4.

47. The method of claim 46, wherein the ratio of glucoamylase to AsAA is about 96:4.

48. The feed additive composition of any one of claims 38-47, wherein the at least one glucoamylase and/or at least one AsAA has at least about 20% activity at pH less than or equal to about 2.5.

49. The feed additive composition of any one of claims 38-47, wherein the at least one glucoamylase and/or at least one AsAA has at least about 20% activity at pH less than or equal to about 3 for at least about 60 minutes.

50. The feed additive composition of any one of claims 38-49, further comprising adding at least one hemicellulase as a feed additive to the feed.

51. The feed additive composition of any one of claims 38-50, further comprising adding betaine as a feed additive to the feed.

52. The feed additive composition of any one of claims 38-51, further comprising adding at least one essential oil as a feed additive to the feed.

53. The feed additive composition of claim 52, wherein the essential oil comprises cinnamaldehyde and/or thymol.

54. The feed additive composition of any one of claims 38-53, further comprising adding at least one direct fed microbial (DFM) as a feed additive to the feed.

55. The method of claim 54, wherein the direct fed microbial is a Megasphaera sp., Bacillus sp., a Propionibacterium sp., and/or an Enterococcus sp.


SEQUENCE LISTING (73 sequences)

SEQ ID NO: 1 - DNA, 2414 bp, Acidomyces richmondensis BFW
  source 1..2414: mol_type = other DNA
atgagggttgctacatttttaggaactctactagccagtcaggttactgtgtctgctctatctcctgctggttggcgggagcagtccatctatcaggttgtcacagatcgcttcgcgctcacctctggagggtcttcaccatcgtgcacttcagacttccaactctcaatcttctgcaatggaacatttcgaggaataattgacaaattggattacatccaaggcatgggcttcaccgccgtgagttatccgcagagatagtgtttagaaatatattaatattgctgttagatgtggatctccccaatcgtcgaaaatatcaaaggcggcacaccagatggttatactgaggatggttctgcttatcatggctactgggcgcaagatatatatgctatcaacccgcactttggtacccccgaagatcttgtggcgttggcagaggctttgcacaatcgcggaatggtacgaagtcaacgcgttttaatcaattgaacttatactaattaagcaattagtatctcatggttgacatcgtagtcaaccacatgggatattattgtggcaaagcagaagacggtcattgtggaccgcagggcacgattgactattcaatttataacccgttcaattcagagaaatatttccatcctttttgtgagatcaactacaacaatgcaacgagtatcttggtggtacgtcagaaagcaatatgtataaacaccacaagtgaagcttaccgttgcagtgttgggaaggtgacgagatcgtaccgctaccggatttgcgcaccgagaataccactgtgcaatccagtgcgtgatctgcatcagatccattgaaaatatgctaaaagttttccagtgttcgatacgtggattaccgaacttgtaaagaattatacaagtaagtcccatcgaaaccatcattagcagtgacatgggaagatgataaggaccatttcacctcgaaagaaatctcatttgaaacccatactagtcgacacttgtgagcaggatcagcagctaacgaaggtgaagttgatggactccgtatcgatagtttgcagcaatctggatctttcttcttcccgtccttttataaggccgcgggagagctatacatggttggagaagtctttcaggaatacccagctaaagtttgtccatatcagcaagatgggatgcctggtgtcttgaactatccaatgtgcgtcctttggcccgactttgccttcttagcgactcttggtaacagtctgtaggttttattacatcaccgatgccttccaggatagcacgggtagcatgagtgggcttgccgaaggcatctcttatatgcaaggcaattgcagtgacacaacattgctaggaagtttcctcgagaatcaggataatccccgctttccatcgtaagcactcaagtttctataatcaaggtttaagctctgactagcacacagatataccgaagacattactcgtgctaagaatgccattgcgttcactatgcttcaagatggaattcctatcatctactatggccaagagcagcatctcaacggctctggcgtgcctcaaaatcgtgaagcgctatggtcttcaggaggctacaatactgcatctatgctctataacatgataaaagaagttaatgctatcagaacgcaggccattcatgtttctcaaagtttcgtcacagagaaaataaccacaccttactacgacaaccacacgatcgttacccgcaaaggcgagaccggctcgcaaataatcggagtatattccaatctcggcgaaaatggtgcttattataatttatcactcaaatcagctgttactggttttgggaacgaagaaaaaatcatggagatcctgacatgcaccttgctgacgaccagttctgatggtgagattatcgtgccaatgggtgagggacaacctaaaatactatttccgttcgcaagactcagtggcagctcgatttgtaagagttataagagtaagctaatcgatatatcacttttacttgtgtagattcaagctgatcaaagatgctccgtagcaatcagcccagtcggaatagactgtgagccagctacaattacttttgacatcctggtaaacactacctacggcgagtctgtcgttgttgctggtgatatacctgaattaggagattggaacgtgcccgatggtctttatctcaatgctagcaagtatacgactgaaaatcctctttggaccggaactgcgacaggcattgatccccaggaagttttccaatggaaacccgttgtcatactaacaaatgggagctatgtatatgcccctggtgacaatattgaaacggtcgctcctgggccaggttgtaatccgacggcgacgataaaatacgttttttctggccagtga

SEQ ID NO: 2 - DNA, 1902 bp, synthetic construct
  source 1..1902: mol_type = other DNA
atgcgagtcgctacctttctcggcaccctcctcgccagccaggtcaccgtcagcgctcttagccctgccggctggcgcgagcagagcatctaccaggtcgtcaccgaccgcttcgccctcacttctggcggcagcagccctagctgcaccagcgacttccagctcagcatcttctgcaacggcaccttccgcggcatcatcgacaagctcgactacatccagggcatgggcttcaccgccatgtggatcagccccatcgtcgagaacatcaagggcggcacccccgacggctacaccgaggatggctctgcctaccacggctactgggcccaggacatctacgccatcaacccccacttcggcacgcccgaggatctcgtcgctctcgccgaggcccttcacaaccgcggcatgtacctcatggtcgacatcgtcgtcaaccacatgggctactactgcggcaaggccgaggacggccattgcggccctcagggcaccatcgactacagcatctacaaccccttcaacagcgagaagtacttccaccccttctgcgagatcaactacaacaacgccaccagcatcctcgtctgctgggagggcgacgagatcgtccctctccctgacctccgcaccgagaacaccaccgtccagagcatgttcgacacctggatcaccgagctggtcaagaactacaccatcgacggcctccgcatcgacagcctccagcagagcggctcgttcttcttcccgtccttctacaaggccgctggcgagctgtacatggtcggcgaggtctttcaggagtaccccgccaaggtctgcccctaccagcaggacggcatgcccggcgtcctcaactaccccatgttctactacatcaccgacgccttccaggacagcaccggcagcatgtctggcctcgccgagggcatcagctacatgcagggcaactgcagcgacacgaccctgctcggcagcttcctcgagaaccaggacaacccccgcttccccagctacactgaggacatcacccgcgccaagaacgccattgccttcaccatgctccaggacggcatccccatcatctactacggccaggagcagcacctcaacggcagcggcgtccctcagaaccgcgaggctctctggtctagcggcggctacaacaccgccagcatgctctacaacatgatcaaggaggtcaacgccatccgcacccaggccatccacgtcagccagagcttcgtcaccgagaagatcaccaccccctactacgacaaccacaccatcgtcacccgcaagggcgagactggcagccagatcatcggcgtctacagcaacctcggcgagaacggcgcctactacaacctcagcctcaagagcgccgtcaccggcttcggcaacgaggagaagatcatggagatcctgacctgcaccctcctcaccacctccagcgacggcgagatcatcgtccccatgggcgagggccagcccaagatcctcttcccgtttgcccgcctcagcggcagcagcatctgcaagagctacaagaccatcagccccgtcggcatcgactgcgagcccgccaccatcaccttcgacatcctggtcaacaccacctacggcgagagcgtcgtcgtggccggcgacattcctgagctgggcgactggaacgtccccgacggcctctacctcaacgccagcaagtacaccaccgagaaccccctctggaccggcaccgccaccggcattgacccccaggaggtgttccagtggaagcccgtcgtcatcctcaccaacggcagctacgtctacgcccctggcgacaacatcgagactgtcgcccctggccctggctgcaaccccaccgccacgatcaagtacgtctttagcggccag

SEQ ID NO: 3 - protein, 634 aa, Acidomyces richmondensis BFW
MRVATFLGTLLASQVTVSALSPAGWREQSIYQVVTDRFALTSGGSSPSCTSDFQLSIFCNGTFRGIIDKLDYIQGMGFTAMWISPIVENIKGGTPDGYTEDGSAYHGYWAQDIYAINPHFGTPEDLVALAEALHNRGMYLMVDIVVNHMGYYCGKAEDGHCGPQGTIDYSIYNPFNSEKYFHPFCEINYNNATSILVCWEGDEIVPLPDLRTENTTVQSMFDTWITELVKNYTIDGLRIDSLQQSGSFFFPSFYKAAGELYMVGEVFQEYPAKVCPYQQDGMPGVLNYPMFYYITDAFQDSTGSMSGLAEGISYMQGNCSDTTLLGSFLENQDNPRFPSYTEDITRAKNAIAFTMLQDGIPIIYYGQEQHLNGSGVPQNREALWSSGGYNTASMLYNMIKEVNAIRTQAIHVSQSFVTEKITTPYYDNHTIVTRKGETGSQIIGVYSNLGENGAYYNLSLKSAVTGFGNEEKIMEILTCTLLTTSSDGEIIVPMGEGQPKILFPFARLSGSSICKSYKTISPVGIDCEPATITFDILVNTTYGESVVVAGDIPELGDWNVPDGLYLNASKYTTENPLWTGTATGIDPQEVFQWKPVVILTNGSYVYAPGDNIETVAPGPGCNPTATIKYVFSGQ

SEQ ID NO: 4 - protein, 615 aa, Acidomyces richmondensis BFW
LSPAGWREQSIYQVVTDRFALTSGGSSPSCTSDFQLSIFCNGTFRGIIDKLDYIQGMGFTAMWISPIVENIKGGTPDGYTEDGSAYHGYWAQDIYAINPHFGTPEDLVALAEALHNRGMYLMVDIVVNHMGYYCGKAEDGHCGPQGTIDYSIYNPFNSEKYFHPFCEINYNNATSILVCWEGDEIVPLPDLRTENTTVQSMFDTWITELVKNYTIDGLRIDSLQQSGSFFFPSFYKAAGELYMVGEVFQEYPAKVCPYQQDGMPGVLNYPMFYYITDAFQDSTGSMSGLAEGISYMQGNCSDTTLLGSFLENQDNPRFPSYTEDITRAKNAIAFTMLQDGIPIIYYGQEQHLNGSGVPQNREALWSSGGYNTASMLYNMIKEVNAIRTQAIHVSQSFVTEKITTPYYDNHTIVTRKGETGSQIIGVYSNLGENGAYYNLSLKSAVTGFGNEEKIMEILTCTLLTTSSDGEIIVPMGEGQPKILFPFARLSGSSICKSYKTISPVGIDCEPATITFDILVNTTYGESVVVAGDIPELGDWNVPDGLYLNASKYTTENPLWTGTATGIDPQEVFQWKPVVILTNGSYVYAPGDNIETVAPGPGCNPTATIKYVFSGQ

SEQ ID NO: 5 - protein, 639 aa, Aspergillus niger
MSFRSLLALSGLVCTGLANVISKRATWDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEVNGSSFFTIAVQHRALVEGSAFATAVGSSCSWCDSQAPEILCYLQSFWTGSFILANFDSSRSAKDANTLLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDATGTYSSSSSTYSSIVDAVKTFADGFVSIVETHAASNGSMSEQYDKSDGEQLSARDLTWSYAALLTANNRRNVVPSASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 6 - protein, 484 aa, Aspergillus niger
LSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRLYVE

SEQ ID NO: 7 - protein, 552 aa, Wolfiporia cocos
QTTSVTSYIASESPIAKAGVLANIGADGSLSSGAYSGIVIASPSTVNPNYLYTWTRDSSLTFMELINQYIYGEDDTLRTLIDEFVSAEATLQQVTNPSGTVSTGGLGEPKFNINETAFTGPWGRPQRDGPALRATAIMAYATYLYENGNTSYVTDTLWPIIELDLGYVAEYWNESTFDLWEEIDSSSFFTTAVQHRALRAGVTFANLIGETSDVSNYQENADDLLCFLQSYWNPTGSYVTANTGGGRSGKDANTLLASIHTFDPDAGCNATTFQPCSDKALSNHKVYVDSFRSLYAINDDISSDAAVATGRYPEDVYYNGNPWYLCTLAAAEQLYDSLIVWKAQGYIEVTSLSLAFFQQFDASVSAGTYDSSSDTYTTLLDAVQTYADGFVLMVAQYTPANGSLSEQYAKADGSPTSAYDLTWSFAAALTAFAARDGKTYGSWGAADLSSTCSGSTDTVAVTFEVQYDTQYGENLYITGSVSQLEDWSADDALIMSSADYPTWSITVDLPPSTLIQYKYLTKYNGDVTWEDDPNNEITTPASGSYTQVDSWH

SEQ ID NO: 8 - protein, 634 aa, Bispora sp.
MRVATFLGTLLASQVTVSALSPAGWREQSIYQVVTDRFALTSGGSSPSCTSDFQLSIFCNGTFRGIIDKLDYIQGMGFTAMWISPIVENIKGGTPDGYTEDGSAYHGYWAQDIYAINPHFGTPEDLVALAEALHNRGMYLMVDIVVNHMGYYCGKAEDGHCGPQGTIDYSIYNPFNSEKYFHPFCEINYNNATSILVCWEGDEIVPLPDLRTENTTVQSMFDTWITELVKNYTIDGLRIDSLQQSGSFFFPSFYKAAGELYMVGEVFQEYPAKVCPYQQDGMPGVLNYPMFYYITDAFQDSTGSMSGLAEGISYMQGNCSDTTLLGSFLENQDNPRFPSYTEDITRAKNAIAFTMLQDGIPIIYYGQEQHLNGSGVPQNREALWSSGGYNTASMLYNMIKEVNAIRTQAIHVSQSFVTEKITTPYYDNHTIVTRKGETGSQIIGVYSNLGENGAYYNLSLKSAVTGFGNEEKIMEILTCTLLTTSSDGEIIVPMGEGQPKILFPFARLSGSSICKSYKTISPVGIDCEPATITFDILVNTTYGESVVVAGDIPELGDWNVPDGLYLNASKYTTENPLWTGTATGIDPQEVFQWKPVVILTNGSYVYAPGDNIETVAPGPGCNPTATIEYVFSGQ

SEQ ID NO: 9 - protein, 631 aa, Symbiotaphrina kochii
MKTAVVLGISAWLASVTALSPAGWREQSIYQIVTDRFALTSGGSTPACTTDTQLGMFCNGTFQGIINKLDYIQNMGFTAIWISPIVKNIDGGSPDGYTVDGSAYHGYWAQDIYQINPHFGTPQDLKQLSQSLHQRGMYLMVDIVVNHNGYYCGLAEDGNCGPQGTIDYSIYNPFNSEEYFHPFCEIDYSNATSILDCWEGDEIVPLPDLRTENTTVQAMFDSWIKELVNNYTIDGLRIDSLQQSGSFFFPSFYSASGGLYMVGEVFQEYPAQVCPYQQAGMPGVLNYPMFYYITDAFEDSAGSMSALANGISYMQGNCSDTTLLGSFLENQDNPRFPTTTGDIVRAMNAITFTLLQDGIPITYYGQEQHLNGSGVPENRQALWSSGGYDTTSQLYEMISKVNAIRTHAIEKASSFVTDKIQTPYYDNHTIVTRKGDPGAQIVGVYSNLGENGDYYDLTLTKEETGFGPIEPVMEILTCTLLTTSFSGDLTVPMGGGQPRILFPYLGIIGSQICPGFTLKPPTGPNCSPAEITFEILANTTWGESVVVTGDIPSLGNWDVANGVHLNASEYTAANPLWVGTATGIDPQEVFQWKPVVIQTDGSYDWYDGDNIQDVSPGPGCNPTGTLQVTFY

SEQ ID NO: 10 - protein, 633 aa, Loramyces juncicola
MRTSILFLAAQSTCALALSGADWRQQSIYQVVIDRFSLTNGDLTPKCTTDFELSNYCNGTFQGIISQLNYIQGMGFTSIWISPIVKNIEGGSPNGYTKDGSPYHGYWAQDIYAINPHFGTEQDLKDLASELHDRGMFLMVDIVVNHMGYYCGAAEEGSCGPEGSIDYSVFNPFDDESYFHSFCLIDYNNATSILDCWEGSQNVPLPDLRIEDVSVQKILDSWITDLVKNYSIDGLRIDSLQQSGSFFFPSFSNATGEIYMVGEVFQEYPAQVCPYQQPGMPGVMNYPMYYYINDAFKSSSGNMKALSEGIAYMQGNCTDTTLLGSFLENQDNARFPSYTGDIVRAANAITFTLLQDGIPITYYGQEQHLNGTGTPYNREALWSTGGYNTSSTLYKLISQVNAIRTKASELSSNFLTSKIKTPYTDDHTIVTCKGEAGNQIIGVYSNLGENGAHYNLTLSKTVTGFNDSQVIVEVLSCTLSTTSPSGDLTLPMGAGEPKVLFPYSVLQNSSICPNPSSPPTAPGQNCSSASITFNILVNTTYGESVVVAGNIPALGNWNVPSGLSLSADKYTAENPLWSGTATGIDPQAAFQWKPVVVKTDGSYVYLPENNLMDVAPGPGCIPSGTIGYKWLSS

SEQ ID NO: 11 - protein, 637 aa, Kockovaella imperatae
MMLLTPILLGAASVVVALSPEQWRAQSIYQVVTDRFALDGGALSPTCTGDFELSIFCNGSFYGLIDKLQYIKDMGFTALWVSPVVENIEGGTPDGYTKDGSPYHGYWAKDIYSINDHFGGSDGLKALSQALHNNDMYLMVDVVVNHMGYYCGAALDGHCGPQGTVNYSIYNPFNSEKYFHPFCEIDYNNATSILDCWEGSENVPLPDLRTEDTVVQQMFDSWISGLVSDYSIDGLRIDSLQQSGSFFFPSFFNATGGMYMVGEVFQEYPKRVCSYQQEGMPGVLNYPMFYYVTDAFSTSDGSMSGLANGISYMRGNCSDVTLLGSFLENQDNPRFPSLTEDITRSMNAITFVMLQDGIPIVYYGQEQHLNGGEVPYNREALWSTGGYNQSAPLYEMITSINKARTAAIQQDASFVTDEMQIPYYDDHVIVARKGRSGDQIVSIYDNRGSSGASSGLTLSQTDTGFSSAEQVMDILSCEQYTTASDGSLFMPLGGGLPKALVTQSVLESSGLCTTYTADSVAGPTSVGSSTTCANATVVFDIYVTTSYGQSVVIAGDISALGDWTPANGLKLSANGYTADSPLWTGSAQVDPGTVFQWKPVIIETNESEDWYSGDNLQNASQAGCHPQSTTILLDWTS

SEQ ID NO: 12 - protein, 602 aa, Saitozyma flava
AGLTPAEWRSQSIYQVVTDRFALDNGGNSPSCSGQSELNLYCNGTFAGIIDKLDYIQNMGFTAIWISPVVKNIDGGSPNGYTPDGSAYHGYWAQDIYEINPHFGGASGLTDLSNALHSRGMYLMVDVVVNHMAYYCGTDGGCGPGNSVNYGSFTPFNSESYFHPFCEIDYNNRTSILDCWEGDEIVPLVDLRTEDSDVQSIFNSWISNLIQTYNIDGLRIDSLQQSGSFFFPGFNQAAGGMYMVGEVFNGSPSYVCPYQQAGMPGVLNYPMFFYITNAFQTSSGSMSQLAQGISAMQSDCSDTTLLGSFLENQDNPRFPSQTSDLTRAQNAIAFTMLQDGIPITYYGQEQHLSGSGVPLNREALWTSGGYDTSSPLYEMITTVNQLRTLAIKQNGGFVTYKIQVPYTDSNHIVTRKGNSGYQIVGVYTNVGSAGASSTLSISSSETGFQASEPVMDVLSCTLYHTGTDGSLSFTMTGGLPRVFYNATALAESSLCTTYTTASPPPGGCSAGTVVFDVYVQTQYGQSVVIAGNIPQLGNWSPANGLNLNANQYTASSPKWTGTITGVAPGTTFQWKPIVVTNGNDNWYPGNNQQATTGSACSS

SEQ ID NO: 13 - protein, 631 aa, Cryptococcus sp.
MAPVRSLAGALLASLGLVAGLSPAEWRSQSIYQVVTDRFALDNGGNSPSCSGQSELNLYCNGTFAGIIDKLDYIQNMGFTAIWISPVVKNIDGGSPNGYTPDGSAYHGYWAQDIYEINPHFGGASGLTDLSNALHSRGMYLMVDVVVNHMAYYCGTDGGCGPGNSVNYGSFTPFNSESYFHPFCEIDYNNRTSILDCWEGDEIVPLVDLRTEDSDVQSIFNSWISNLIQTYNIDGLRIDSLQQSGSFFFPGFNQAAGGMYMVGEVFNGSPSYVCPYQQAGMPGVLNYPMFFYITNAFQTSSGSMSQLAQGISAMQSDCSDTTLLGSFLENQDNPRFPSQTSDLTRAQNAIAFTMLQDGIPITYYGQEQHLSGSGVPLNREALWTSGGYDTSSPLYEMITTVNQLRTLAIKQNGGFVTYKIQVPYTDSNHIVTRKGNSGYQIVGVYTNVGSAGSSSTLSISSSETGFQASEPVMDVLSCTLYHTGTDGSLSFTMTGGLPRVFYNATALAESSLCTTYTTASPPPGGCSAGTVVFDVYVQTQYGQSVVIAGNIPQLGNWSPANGLNLNANQYTASSPKWTGTITGVAPGTTFQWKPIVVTNGNDNWYPGNNQQATTGSACSSPAADIEFTWSS

SEQ ID NO: 14 - protein, 631 aa, Saitozyma flava
MAPVRSLAGALLASLGLVAGLSPAEWRSQSIYQVVTDRFALDNGGNSPSCSGQSELNLYCNGTFAGIIDKLDYIQNMGFTAIWISPVVKNIDGGSPNGYTPDGSAYHGYWAQDIYEINPHFGGASGLTDLSNALHNRGMYLMVDVVVNHMAYYCGTNGGCGPGNSVNYGSFTPFNSESYFHPFCEIDYNNRTSILDCWEGDEIVPLVDLRTEDSDVQSIFNSWISNLIQTYNIDGLRIDSLQQSGSFFFPGFNQAAGGMYMVGEVFNGNPSYVCPYQQAGMPGVLNYPMFFYITNAFQTSSGSMSQLAQGISAMQSDCSDTTLLGSFLENQDNPRFPSQTNDLTRAQNAIAFTMLQDGIPITYYGQEQHLSGSGVPLNREALWTSGGYDSSSPLYKMITTVNQLRTLAIKQNGGFVTYKIQVPYTDSNHIVTRKGNSGYQIVGVYTNVGSAGASSTLSLSSSETGFQASEPVMDVLSCTLYHTGSDGSLSFTMTGGLPRVFYNATALAESSLCTTYTTATPPPGGCSAGTVVFDVYVQTQYGQSVVIAGNIPQLGNWSPANGLNLNANQYTASSPKWTGTITGVAPGTTFQWKPIVVTNGNDNWYTGSNQQATTGSACSSPATDIEFTWSS

SEQ ID NO: 15 - protein, 615 aa, Bispora sp.
LSPAGWREQSIYQVVTDRFALTSGGSSPSCTSDFQLSIFCNGTFRGIIDKLDYIQGMGFTAMWISPIVENIKGGTPDGYTEDGSAYHGYWAQDIYAINPHFGTPEDLVALAEALHNRGMYLMVDIVVNHMGYYCGKAEDGHCGPQGTIDYSIYNPFNSEKYFHPFCEINYNNATSILVCWEGDEIVPLPDLRTENTTVQSMFDTWITELVKNYTIDGLRIDSLQQSGSFFFPSFYKAAGELYMVGEVFQEYPAKVCPYQQDGMPGVLNYPMFYYITDAFQDSTGSMSGLAEGISYMQGNCSDTTLLGSFLENQDNPRFPSYTEDITRAKNAIAFTMLQDGIPIIYYGQEQHLNGSGVPQNREALWSSGGYNTASMLYNMIKEVNAIRTQAIHVSQSFVTEKITTPYYDNHTIVTRKGETGSQIIGVYSNLGENGAYYNLSLKSAVTGFGNEEKIMEILTCTLLTTSSDGEIIVPMGEGQPKILFPFARLSGSSICKSYKTISPVGIDCEPATITFDILVNTTYGESVVVAGDIPELGDWNVPDGLYLNASKYTTENPLWTGTATGIDPQEVFQWKPVVILTNGSYVYAPGDNIETVAPGPGCNPTATIEYVFSGQ

SEQ ID NO: 16 - protein, 613 aa, Symbiotaphrina kochii
LSPAGWREQSIYQIVTDRFALTSGGSTPACTTDTQLGMFCNGTFQGIINKLDYIQNMGFTAIWISPIVKNIDGGSPDGYTVDGSAYHGYWAQDIYQINPHFGTPQDLKQLSQSLHQRGMYLMVDIVVNHNGYYCGLAEDGNCGPQGTIDYSIYNPFNSEEYFHPFCEIDYSNATSILDCWEGDEIVPLPDLRTENTTVQAMFDSWIKELVNNYTIDGLRIDSLQQSGSFFFPSFYSASGGLYMVGEVFQEYPAQVCPYQQAGMPGVLNYPMFYYITDAFEDSAGSMSALANGISYMQGNCSDTTLLGSFLENQDNPRFPTTTGDIVRAMNAITFTLLQDGIPITYYGQEQHLNGSGVPENRQALWSSGGYDTTSQLYEMISKVNAIRTHAIEKASSFVTDKIQTPYYDNHTIVTRKGDPGAQIVGVYSNLGENGDYYDLTLTKEETGFGPIEPVMEILTCTLLTTSFSGDLTVPMGGGQPRILFPYLGIIGSQICPGFTLKPPTGPNCSPAEITFEILANTTWGESVVVTGDIPSLGNWDVANGVHLNASEYTAANPLWVGTATGIDPQEVFQWKPVVIQTDGSYDWYDGDNIQDVSPGPGCNPTGTLQVTFY

SEQ ID NO: 17 - protein, 616 aa, Loramyces juncicola
LSGADWRQQSIYQVVIDRFSLTNGDLTPKCTTDFELSNYCNGTFQGIISQLNYIQGMGFTSIWISPIVKNIEGGSPNGYTKDGSPYHGYWAQDIYAINPHFGTEQDLKDLASELHDRGMFLMVDIVVNHMGYYCGAAEEGSCGPEGSIDYSVFNPFDDESYFHSFCLIDYNNATSILDCWEGSQNVPLPDLRIEDVSVQKILDSWITDLVKNYSIDGLRIDSLQQSGSFFFPSFSNATGEIYMVGEVFQEYPAQVCPYQQPGMPGVMNYPMYYYINDAFKSSSGNMKALSEGIAYMQGNCTDTTLLGSFLENQDNARFPSYTGDIVRAANAITFTLLQDGIPITYYGQEQHLNGTGTPYNREALWSTGGYNTSSTLYKLISQVNAIRTKASELSSNFLTSKIKTPYTDDHTIVTCKGEAGNQIIGVYSNLGENGAHYNLTLSKTVTGFNDSQVIVEVLSCTLSTTSPSGDLTLPMGAGEPKVLFPYSVLQNSSICPNPSSPPTAPGQNCSSASITFNILVNTTYGESVVVAGNIPALGNWNVPSGLSLSADKYTAENPLWSGTATGIDPQAAFQWKPVVVKTDGSYVYLPENNLMDVAPGPGCIPSGTIGYKWLSS

SEQ ID NO: 18 - protein, 620 aa, Kockovaella imperatae
LSPEQWRAQSIYQVVTDRFALDGGALSPTCTGDFELSIFCNGSFYGLIDKLQYIKDMGFTALWVSPVVENIEGGTPDGYTKDGSPYHGYWAKDIYSINDHFGGSDGLKALSQALHNNDMYLMVDVVVNHMGYYCGAALDGHCGPQGTVNYSIYNPFNSEKYFHPFCEIDYNNATSILDCWEGSENVPLPDLRTEDTVVQQMFDSWISGLVSDYSIDGLRIDSLQQSGSFFFPSFFNATGGMYMVGEVFQEYPKRVCSYQQEGMPGVLNYPMFYYVTDAFSTSDGSMSGLANGISYMRGNCSDVTLLGSFLENQDNPRFPSLTEDITRSMNAITFVMLQDGIPIVYYGQEQHLNGGEVPYNREALWSTGGYNQSAPLYEMITSINKARTAAIQQDASFVTDEMQIPYYDDHVIVARKGRSGDQIVSIYDNRGSSGASSGLTLSQTDTGFSSAEQVMDILSCEQYTTASDGSLFMPLGGGLPKALVTQSVLESSGLCTTYTADSVAGPTSVGSSTTCANATVVFDIYVTTSYGQSVVIAGDISALGDWTPANGLKLSANGYTADSPLWTGSAQVDPGTVFQWKPVIIETNESEDWYSGDNLQNASQAGCHPQSTTILLDWTS

SEQ ID NO: 19 - protein, 602 aa, Saitozyma flava; identical to SEQ ID NO: 12

SEQ ID NO: 20 - protein, 611 aa, Cryptococcus sp.
LSPAEWRSQSIYQVVTDRFALDNGGNSPSCSGQSELNLYCNGTFAGIIDKLDYIQNMGFTAIWISPVVKNIDGGSPNGYTPDGSAYHGYWAQDIYEINPHFGGASGLTDLSNALHSRGMYLMVDVVVNHMAYYCGTDGGCGPGNSVNYGSFTPFNSESYFHPFCEIDYNNRTSILDCWEGDEIVPLVDLRTEDSDVQSIFNSWISNLIQTYNIDGLRIDSLQQSGSFFFPGFNQAAGGMYMVGEVFNGSPSYVCPYQQAGMPGVLNYPMFFYITNAFQTSSGSMSQLAQGISAMQSDCSDTTLLGSFLENQDNPRFPSQTSDLTRAQNAIAFTMLQDGIPITYYGQEQHLSGSGVPLNREALWTSGGYDTSSPLYEMITTVNQLRTLAIKQNGGFVTYKIQVPYTDSNHIVTRKGNSGYQIVGVYTNVGSAGSSSTLSISSSETGFQASEPVMDVLSCTLYHTGTDGSLSFTMTGGLPRVFYNATALAESSLCTTYTTASPPPGGCSAGTVVFDVYVQTQYGQSVVIAGNIPQLGNWSPANGLNLNANQYTASSPKWTGTITGVAPGTTFQWKPIVVTNGNDNWYPGNNQQATTGSACSSPAADIEFTWSS

SEQ ID NO: 21 - protein, 611 aa, Saitozyma flava
LSPAEWRSQSIYQVVTDRFALDNGGNSPSCSGQSELNLYCNGTFAGIIDKLDYIQNMGFTAIWISPVVKNIDGGSPNGYTPDGSAYHGYWAQDIYEINPHFGGASGLTDLSNALHNRGMYLMVDVVVNHMAYYCGTNGGCGPGNSVNYGSFTPFNSESYFHPFCEIDYNNRTSILDCWEGDEIVPLVDLRTEDSDVQSIFNSWISNLIQTYNIDGLRIDSLQQSGSFFFPGFNQAAGGMYMVGEVFNGNPSYVCPYQQAGMPGVLNYPMFFYITNAFQTSSGSMSQLAQGISAMQSDCSDTTLLGSFLENQDNPRFPSQTNDLTRAQNAIAFTMLQDGIPITYYGQEQHLSGSGVPLNREALWTSGGYDSSSPLYKMITTVNQLRTLAIKQNGGFVTYKIQVPYTDSNHIVTRKGNSGYQIVGVYTNVGSAGASSTLSLSSSETGFQASEPVMDVLSCTLYHTGSDGSLSFTMTGGLPRVFYNATALAESSLCTTYTTATPPPGGCSAGTVVFDVYVQTQYGQSVVIAGNIPQLGNWSPANGLNLNANQYTASSPKWTGTITGVAPGTTFQWKPIVVTNGNDNWYTGSNQQATTGSACSSPATDIEFTWSS

SEQ ID NO: 22 - protein, 637 aa, Aspergillus welwitschiae
MRLSTSSLFLSVSLLGKLALGLSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISDLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGSNSSTTTTTTATSSSTATSKSASTSSTSTACTATSTSLAITFEELVTTTYGEEIYLSGSISQLGDWDTSDAVKMSADDYTSSNPEWSVTVTLPVGTTFEYKFIKVESDGSVTWESDPNREYTVPECGSGETVVDTWR

SEQ ID NO: 23 - protein, 636 aa, Aspergillus brasiliensis
MRLSTSILALSVSLLGKLALGLSAAEWRTQSIYFLLTDRFGRTDNSTTATCNTGDQIYCGGSWQGIINHLDYIQGMGFTAIWISPITEQLPQDTSDGEAYHGYWQQKIYDVNSNFGTADDLKSLSDALHARGMYLMIDVVPNHMGYAGSGNDVDYSVFDPFDSSSYFHPYCLITDWDNLTMVQDCWEGDTIVSLPDLNTTETVVRTIWYDWVADLVSNYSVDGLRIDSVLEVEPDFFPGYQEAAGVYCVGEVDNGNPALDCPYQDYLDGVLNYPIYWQLLYAFESSSGSISDLYNMIKSVASDCSDPTLLGNFIENHDNPRFAYYTSDYSQAKNVLSYIFLSDGIPIVYAGEEQHYSGGDVPYNREATWLSGYDTSAELYTWIATTNAIRKLAIAADSSYITYANDPIYTDSNTIAMRKGTSGSQVITVLSNKGSSGSSYTLTLSGSGYTSGTKLIEAYTCTSVTVDSNGDIPVPMASGLPRVLLPASVVDDSSLCGGSGSSTSTTTSTATATTTSKTSTTSSSSSSSSCTASATAIPITFEELVTTTYGEEIYLSGSISQLGDWDTSDAVKLSADDYTSSNPEWSVTVTLPVGTTFEYKFIKVESGGSVTWESDPNREYTVPECGSGETVVDTWR

SEQ ID NO: 24 - protein, 630 aa, Aspergillus sclerotiicarbonarius
MKLSSSSLLLSAPLFGKLALGLSAAEWRSQSIYFLLTDRFGRTDNSTTATCDTGEQIYCGGSWQGVINHLDYIQGMGFTAIWISPVTEQLPQDTADGEAYHGYWQQEIYTVNSNFGTADDLKALSDALHARGMYLMVDVVPNHMGYAGDGDDVDYSVFDPFDSSSYFHPYCLITDWSNLTMVRDCWEGDTIVSLPDLNTTETVVRTIWYDWVADLVANYSVDGLRIDSALEVEPDFFPGYQEAAGVYCVGEVDNGDPTLDCPYQEYLDGVLNYPIYWQLLYAFESSSGSISDLYNMIKSVASDCSDPTLLGNFIENHDNPRFAYYTSDYSEAKNVLSYIFLSDGIPIVYAGEEQHYNGGDVPYNREATWLSGYDTSAELYTWIATTNAIRKLAIEADSDYITYANDPFYTDSNTIAMRKGTSSQQVITVLSNKGSSGSSYTLTLSGSGYTSGTELIEAYTCTSVTVDSNGDIPVPMDSGLPRVFLPASSINSSSSSLCGGSSTTTTTTTSTSPSTATSSSTATATTTCTTLPITFIESVTTTYGEEIYLSGSITQLGDWDTSDAVLLSADNYTSSNPEWYVEVSLPTGTSFEYKFIKKESDGSVTWESDPNREYTVPEECGGVTVDDSWR

SEQ ID NO: 25 - protein, 629 aa, Talaromyces rugulosus
MVAIAKRAAFLASLFLGQSVVALTPAEWRSQSIYFLLTDRFGREDNSTTASCDLSERIYCGGSWQGIINHLDYIQGMGFTAIWITPVTEQLPQDTGEGEAYHGYWQQEIYSVNSNYGTAADLLALSEALHDRGMYLMVDVVANHMGYAGAGDSVDYSVFSPFDSSSYFHSYCLISDYSNQENVEDCWLGDTTVSLPDLNTDLTSVQTLWYDWVADLVSNYSIDGLRIDTVKHVQESFWPGYNSAAGVYCVGEVFDGDPAYTCPYQNYLDAVLNYPIYYQLLYAFESSSGSISGLYDMISSVASDCADPTLLGNFIENHDNPRFASYTDDYSQAKNVISFVFLSDGIPIIYAGQEQHYSGGSDPENREATWLSGYSTTAELYQFIATTNKIRALAVSSDSSYITTKNDPFYTDSNTIAMQKGSSGSQVVTVLSNMGASGSSYTLTLSGSGYASGTELMEMYTCTSVTVDSSGNIAVPMASGLPRVFMLASSAGSLCGSSTTTTTSKITSTTSTSSTSSTSTSCTQTTDVPVLFKELVTTTYGEDIYISGSISELGDWDTANAVALSSSGYTSSNPLWQVVVSIPAGTSFEYKFFETGSSSSVTWESDPNRSYTVPTACSGSTATVTATWR

SEQ ID NO: 26 - protein, 634 aa, Penicillium rolfsii
MKTLGLAAIFLAKAVAGLTPAEWRSQSIYFLLTDRFGRTDNSTTATCNVSDRIYCGGSWQGIINHLDYIQGMGFTAIWITPVTEQLSQDTGDGEAYHGYWQQEIYNVNTNYGTAADLLALSKALHSRGMYLMVDVVANHMGYDGAGNTVDYSVFNPFDSSSYFHSYCEITDYSNQTNVEDCWLGDTTVSLPDLDTTLSSVQTIWYNWVTGLVSNYSIDGLRIDTVKHVQKSFWPGYNSAAGVYCMGEVFDGDPAYTCAYQSYLDGILNYPVYYQLLYAFESTSGSISNLYNMINSVASDCSDPTLLGNFIENHDNPRFASYTSDYSQAKNVISFIFFSDGIPIVYAGQEQHYSGGSDPANREATWLSGYDKTAQLYTYITTTNKIRALAISKDSAYITSKNTAFYSDSNTIAMKKGSSGSQVITVLSNRGSSGSSYTLSLSGSGYSSGKKLMEMYTCTAVTVDSSGNIAVPMASGLPRVYMLASSACSICSSTCSTTTSTASSTSSSTTTASTTLKTTASSASTSCTQATALPVLFKEIVTTSYGQNIYISGSISQLGSWDTSSAVALSADQYTSSNHLWYVVVTIPVGTSFQYKFIEETSGYSTITWESDPNRSYTVPTGCVGSTATVTATWR

SEQ ID NO: 27 - protein, 617 aa, Aspergillus felis
MKWTSSLLPLSLSLLGQAAHALTPAEWRSQSIYFLLTDRFGRQDNSTTAACDVTQRLDYIQGMGFTAVWITPVTEQFYEDTGDGTSYHGYWQQNIYEVNSNYGTAQDLKSLANALHARGMYLMVDVVANHMGYDGAGTSVDYSVFNPFNSSSYFHPYCLISDYSNQTNVEDCWLGDTTVSLPDLNTTETTVRTIWYDWVKALVANYSIDGLRIDTVKHVEKAFWPDYNSAAGVYCVGEVFSGDPAYTCPYQNYLDGVLNYPIYYQLLYAFESTSGSISNLYNMISSVASDCADPTLLGNFIENHDNPRFASYTSDYSQAKNVVSFIFFSDGIPIVYAGQEQHYSGGADPANREAVWLSGYSTSATLYSWIASTNKIRKLAISKDSAYVTSKNNPFYYDPNTLAMRKGSTAGAQVITVLSNKGSSGSSYTLSLSGTGYSAGATLVEMYTCTTLTVDSSGNLAVPMASGLPRVLVPASWVSGSGLCGGSVSTTATTTTSATTTTTATTTTACTSATALPILFEELVTTTYGENIYLTGSISQLSNWDTSSAIALSAGNYTSSNPEWYVTVTLPIGTSFQYKFFKKESDGSITWESDPNRSYTVPTGCAGTTVTVSDTWR

SEQ ID NO: 28 - protein, 612 aa, Aspergillus hiratsukae
MKWNLLLLPLSLSLLGQAAHALTPAEWRTQSIYFLLTDRFGREDNSTTAACDVTERLDYIQGMGFTAIWINPVTEQFYEDTGDGTSYHGYWQQNIYEANHNYGTAQDLKSLADALHARGMYLMVDVVANHMGYDGAGNSIDYSVFTPFDSSSYFHPYCLISDYSNQTNVEDCWLGDTTVSLPDLDTTDAKVRTIWYDWVKGLVANYSIDGLRIDTVKHVEKDFWPGYNDAAGVYCVGEVFSGDPTYTCPYQNYLDGVLNYPIYYELLYAFESPRGSISNLYNMISSVASDCADPTLLGNFIENHDNPRFASYTSDYSQAKNVISFMFFSDGIPIVYAGQEQHYSGGADPANREAVWLSGYSTSATLYSWIASTNRIRKLAISKDTAYITSKNNPFYYDSNTLAMRKGSVAGAQVVTVLSNKGSSGSSYTLSLSGTGYSAGASLVEMYTCTTLTVDSSGNLAVPMASGLPRVLVPSSWVSGSGLCGSGSTTTTTTTTTATATTTACTSATALPILFEEILTTTFGENIYLSGSISQLGNWDTSSAIALSASKYTSSNPEWYVTVTVPVGTSFQYKFFKNVSDGSIVWETDPNRSYTVPSGCGGTTVTLSGTWR

SEQ ID NO: 29 - protein, 616 aa, Aspergillus viridinutans
MKWTSSLLPLSLSLLGQAAHALTPAEWRSQSIYFLLTDRFGREDNSTTAACDVTQRLDYIQGMGFTAIWITPVTEQFYEDTGDGTSYHGYWQQNIYEVNSNYGTAQDLKSLADALHARGMYLMVDVVANHMGYDGAGNSVDYSVFTPFGSSSYFHPYCLISDYSNQTNVEDCWLGDTTVSLPDLDTTDTTVQTICTRTWAWMILTVIVDGLRIDTVKHVEKDFWPGYNDAAGVYCVGEVFSGDPTYTCPYQNYLDGVLNYPIYYQLLYAFESTSGRISNLYNMLSSVASDCADPTLLGNFIENHDNPRFASYTSDYSQAKNVISFMFFSDGIPIVYAGQEQHYSGGADPANREAVWLSGYSTSATLYSWIASTNKIRKLAISKDSAYITSKNNPFYYDSNTLAMRKGSTAGSQVITVLSNKGFSGSSYTLSLSGTGYSAGTTLVEMYTCTSLTVDLSGNLAVPMASGLPLVLVPSSWVSGSGLCGSSVSTTATATSTTKTTIATTTAACTSATALPILFEELVTTTYGENIYLTGSISQLGNWDTSSAIALSASKYTSSNPEWYVTVTLPVGTSLQYKFFKKESDGSIVWESDPNRSYTVPTGCAGTTVTASDTWR

SEQ ID NO: 30 - protein, 501 aa, Aspergillus sclerotialis
MHLLISVVFLLLLQTVSSLSPAEWRSQSIYFLLTDRFGRTDNSTTAECDVEKRLYCGGTWQGIINQLDYIQGMGFTAIWITPVTEQLSQDTGYGEAYHGYWQQKIYNLDSHLGTADDLKALSKALHDRGMYLMVDVVANHMGYDGPGNSVDYSVFDPFDSSSYFHKYCLISNYDDQNDVQNCWLGDTTVSLPDLNTKDAEVRELWYDWVTGLVSNYSIDGLRIDTVKHVEKTFWEGYNQAAGVYCVGEVFDGDPKYTCAYQSYLDAVLNYPIYYPLLNAFKAPGGNMGDLYNMVNSVASHCSDPTVLGNFIENHDNPRFASYTDDYSLAKNAITFMFLSDGIPIIYAGEEQHYSGGSDPGNREAVWLSGYSTDAELYKFIATTNKIRSLVILKDNKYVTSKNVPFYQDDNTFAMRKGSGNSQAITVLSNAGSSSDSYSVTVKGAEYSSGTQLIDLYSCAKVTVGDDGSISLQVESGLPKVLVPASYAGDALCGGSSAPSIA

SEQ ID NO: 31 - protein, 627 aa, Penicillium steckii
MYLIFAALLMLAKGISALSPAEWRSQSIYFLLTDRFGREDNSTTASCDVSNRLDYIQDMGFTAIWITPVTEQLQGDTGYGEAYHGYWQQDIYDVNENYGTAEDLLALSNALHDRGMYLMVDVVANHMGYNGAGNTVDYSVFNPFDSSSYFHSYCLITNYDNQDNVEDCWLGDTTVSLPDLNTDLSSVQTIWYDWISDLVSNYSIDGLRIDTVKHVQKSFWPSYNSAAGVYCVGEIFDGNPAYTCDYQNYMDGVLNYPIYYQLLYAFQSTSGSISDLYNMIGTVASDCKDPTLLGNFIENHDLPRFASFTSDYSLAKNVLSFLFFSDGIPIVYAGQEQHYSGKDDPANREATWLSGYSTSAELYKHISTTNKIRKLAASSDSSYITSRVIIVSSKLDKPKGLTNSMQNTPFYQDSHTIAMKKGSSGSQVITVLSNYGSSGSSYTLTLGSSGYSSGTELMEMYTCTTATVDSSGNIPVPMVSGLPRVLMLASVAKSSGPCASSGSPTTSTASSSSTTMSACTKATALPVLFKETVSTSYGEDIYISGSISRLGSWDASNAVALSADQYTSSNPLWYVSITLPVGTSFEYKFIKKTSGSSTVTWESDPNRSYTVPTACSGSTATVTATWR

SEQ ID NO: 32 - protein, 498 aa, Aspergillus leporis
MVSWWSLVLCGLQVLGPAFAATPAEWRSQSIYFLLTDRFARTDGSTTASCNTEDRKYCGGTWQGIIDKLDYIQGMGFTAIWITPVTGQLPQHTAYGDAYHGYWQQDIYALNSNYGTADDLKALASALHDRGMYLMVDVVANHMGYDGAGDSVDYSVFKPFNSKDYFHNFCLIQNYDDQTQSENCWLGDNSVSLPDLDTTREDVKGLWYDWVEALVSNYSIDGLRIDTVKHVQKDFWPGYNQAAGVYCIGEVLNGDPAYTCPYQNVMDGVLNYPMYYPLLNAFKSTSGSISDLYNMINTVKSTCPDSTLLGTFVENHDNPRFASYTNDMSLAKNVAAFIILADGLPIIYAGQEQHYAGGNDPANREATWFSGYSTTSEIYKLIASSNAIRNHAISTDSGYLTYKNWPVYQDASTIAMRKGTDGSQIITVLSNQGSSGGSYTLSLTGTGYTAGQQLTEITTCSTVTVGLDGKVPLPMEKGLPKVLYPSAKLSGSKVCSSS

SEQ ID NO: 33 - protein, 492 aa, Aspergillus tanneri
MKAGWLLSLASVALAATPADWRSQSIYFLLTDRFARSDGSTTASCNTEDRKYCGGTWKGIIDQLDYIQGMGFTAIWITPVTGQLPQDTGYGEAYHGYWQQDIYSLNDKYGTADDLKALADALHNRHMYLMVDVVANHMGYSGAGDSVDYSVFKPFNSKDYFHPYCPIKDYNDQTQAENCWLGDNTVSLPDLDTTKSEVKDLWYKWVGDLVSNYSIDGLRIDTVKHVQKDFWPGYNKAAGVYCVGEVLNGDTAFTCAYQSVLDGVLNYPMYYPLLNAFKSTSGNIGDLYNMINNVKSNCPDSTLLGTFIENHDNPRFASYTNDMSLAKNVAVFTILADGIPIIYAGQEQHYAGGEDPANREATWLSGYSTKSELYTLIASSNAIRNHAIAQDPKYVTYKNYPIYQDETTIAMRKGSDGSQVVTVLSNKGSSGSSYTLSLGDTGYSSGQNVTEIYTCETLTVGSDGKVSVPMNNGLPRVLYPSEKLKGSSLCSE

SEQ ID NO: 34 - protein, 495 aa, Aspergillus avenaceus
MVSWRSLLLGLQVVAPILAATPADWRSQSIYFLLTDRFARTDGSTSATCNTEDRKYCGGTWKGIIDQLDYIQGMGFTAIWITPVTSQLTQDTAYGEAYHGYWQQDIYSLDSHLGTADDLRALASALHERGMYLMVDVVANHMGYDGAGDSVDYSVFNPFNSKDYFHPFCLIQDYNDQTQSENCWLGDNSVSLPDLDTTTQDVKNIWYDWVEELVSNYSIDGLRVDTVKHVQKDFWPDYNKAAGVYCIGEVLNGDPSYTCPYQEVMDGVLNYPIYYPLLNAFKSTSGNMNDLYNMINTVKSDCPDSTLLGTFIENHDSPRFASYTNDMALAKNVAAFIILADGIPIIYAGQEQHYAGGNDPSNREATWLSGYSTTSDLYKLIASSNAIRNHAVSTDKGYVTYKNWPIYKDTTTIAMRKGTDGSQIITILSNKGASGDGYTLSLDGTGYDAGTELTEILSCSTLTVGSDGKVPVPMEKGLPRVLYPSAKLGDSKICT

SEQ ID NO: 35 - protein, 496 aa, Aspergillus novofumigatus
MPIRWLSLILSCLAGTAFAATPEQWRSQSIYFLLTDRFARTDGSTTASCDTTARKYCGGTWQGIIKQLDYIQGMGFTAIWITPVTEQLPQDTSEGTAYHGYWQQDIYSVNSNYGTADDLKALGSALHDRGMYLMVDVVANHMGYAGAGNSVDYSLFSPFNSQTYFHPLCFISNYDNQTNVENCWLGDNVVPLADLDTTKSDVQKIWYNWVGSLVSNYSIDGLRIDTVKHVQKDFWPGFNDAAGVYCIGEVFDGDPAYTCPYQDVLDGVLNYPTYYPLLKAFQSTSGSMSDLYNMINTVKSQCADSTLLGTFVENHDTPGSHRKYTNDMALAKSAAAFIILSDGIPIIYAGQEQHYSGGTDPANREAVWLSGYSKTSELYKLIASANAIRNHAISKDPEYVTYKNNPIYKDTSTIAMRKGADGAQVITVLSNLGASGSSYTLSLSGTGYDVGQQLTEVFSCATVTVDSDGKVPVAMASGLPRAFYPTAGLNGSTICA

SEQ ID NO: 36 - protein, 498 aa, Aspergillus nomiae
MVAWWSLFLYGLQAAAPALAATPADWRSQSIYFLLTDRFARTDGSTTAACNTEDRKYCGGTWQGIIDKLDYIQGMGFTAIWITPVTGQLPQNTAYGEAYHGYWQQDIYSLNENYGTPDDLKALASALHTRGMYLMVDVVANHMGYDGAGASVDYSVFKPFNSQEYFHPFCLIQNYNDQTQSEDCWLGDNSVSLPDLDTTKDEVKNEWYEWVGSLVSNYSIDGLRVDTVKHVQKDFWPGYNKAAGVYCIGEVLNGDPAYTCPYQDVMDGVLNYPIYYPLLNAFKSTSGSMDDLYNMINTVKSDCPDSTLLGTFVENHDNPRFASYTNDIALAKNVAAFIILNDGIPIIYAGQEQHFAGGNDPANREATWLSGFSTDSEIYKLIASANAIRSHAISIDTGFVTYKNWPIYKDTTTIAMRKGTDGSQVVTILSNKGASGDSYTLSLGNTGYTAGQKLTEVIGCTTVTVGSDGTVPVPMAGGLPRILYPTEKLGDSKICSSA

SEQ ID NO: 37 - protein, 497 aa, Aspergillus flavus
MVSWWSFLCGLQVFGLALAATPAEWRSQSIYFLLTDRFARTDGSTTASCNTEDRKYCGGTWQGIIDKLDYIQGMGFTAIWITPVTGQLPQHTAYGDAYHGYWQQDIYTLNRNYGTADDLKALASALHDRGMYLMIDVVVNHMGYDGAGDSVNYSVFKPFNTKDYFHNFCFIQNFDDQTQSENCWLGDNRVSLPDLDTTREDVKGLWYDWVEAFVSNYSIDGLRVDTVKHVQKDFWRGYNEAAGVYCIGEVFNGDPAYTCPYQNIMDGVLNYPMYYPLLNAFKSTSGSISNLYNMINTVKSTCPDSTLLGTFVENHDNPRFASYTNDMSLAKNVAAFIILADGLPIIYAGQEQHYAGGNDPANRDATWFSGYSTTSELYKLIASLNAIRRHAINTDSGYLSYKNWPIYQDASTIAMRKGTDGSQIITVLSNQGSSGGSYTLSLTGTGYTAGEQLHEIINCSTVTVGSDGKVLLPMEKGLPRVLYPSAKLNGSKVCSSS

SEQ ID NO: 38 - protein, 622 aa, Aspergillus udagawae
MKLLTIALTLPLLGKSVLALNAAEWRSQCIYFLMTDRFARTDGSTTAPCDVGQRGYCGGSWKGIIDHLAYIQGMGFTAIWITPIVEQIPQTTTEGTGFHGYWPQNIYSVNSHFGTADDIRALSKALHDRGMYLMMDGVANHMGFNGPGASNGFSTFTPFNSASYFHSYCPINNYNDQWQVENCYLRDSVVSLTDLNTQSSAVRGIWYDWVKDLVANYSVDGLRIDTAKHVEKDFWSGYTQAAGVYSVGEILHGDPAYTCPYQGYMDGVMNYPIYYQLVNAFKSSSGSISSLSNMISSVASKCKDPTLLGNFIENHDNPRFPSYTSDISQAKSVIAYVFLTDGIPIIYSGQEQHLSGGADPYNREALWLSGYSTNSELYKFIAMTNKIRRLAISKDLNYLPARNNPFYTDSNTIAMKKGSGGSNVITVLTNSGSNAGSYTLNLNSHGYSSGSSLIELYTCSSVQVDSNGNLPVPMSSGLPRVLVPSAWVPGSGLCGASSTTSLATPTVTSPSPGTCAPSTAIPVIFKERVATSYGENIFLSGSIDQLGNWDTSKAVALSASGYTSSNPVWSVKLDLHAGTYFQYKFIKKGQDGSVMWESDPNRSYTLPSGCVETAISIADSWR

SEQ ID NO: 39 - protein, 525 aa, Aspergillus ellipticus
MMEILILFALVATVLGATPADWRSQSIYFLLTDRFARTDNSTTASCNTGARQYCGGTWQGIINQLDYIQGMGFTAIWITPVTGQIPQETGYGEAYHGYWQQDAYALNSHYGTADDLKKLVSALHARGMYLMVDVVANHMAYNGSADTTDYSAYNPFNSKEYFHKICSIEDNNNQTQTEDCWLADSIVSLPDLNTTQTDVKNMWYDWIGSLVSNYSIDGLRIDTVKNVQQNFWPDYNTAAGVYCVGEVFDGDAEYTCPYQNYLDGVLNYPMYYPILRAFQSTSGSISDLYNMINTVKSTCKDSTLLGTFVENHDNPRFANYTSDMSLAKNAAAFTMLADGIPIIYAGQEQHYHGGNDPYNREATWLAKYSTTSPLYELIATSNRIRSHAISQDSGYITYKNYPIYQDNSTLAMRKGYNGSQTITVLSNLGASGKTYNLSLPGTGYTANQQLMEIYTCTNITVDSSGSVSVPMASGLPRVLYPADKLNGSLLCNSTTSSATSLSPLSNSAIPIYLTLAAFVVQLSQL

SEQ ID NO: 40 - protein, 496 aa, Aspergillus sp.
MKPFTYAIAMFLAQTVAALSPAEWRGQSIYFLLTDRFARADNSTTAPCDVTDRVYCGGSWQGIINQLDYIQGMGFTAIWVSPVTEQLPQDTEYGEAYHGYWQQDIYNLNKHFGTPDDLNALADALHERGMYLMVDVVPNHMGYDGPGDSVDYGVFEPFSSSSYFHPFCFIENYDNQDNVEQCWLGDSKVPLPDLDTSREDVQNIWYDWVSGLVSNYSVDGLRLDTAKHVQKNFWPDYNKAAGVYCVGEVLSGDPKYTCPYQEYMDAVLNYPIYYQLLDAFKSTNGPTDKVYNMISRVASSCRDPTLLGNFIENHDAPRFASYTQDLALAKNAIAFLFLTDGIPIVYAGQEQHYSGGDDPFNRGAIWLSGYSTEAALYRFIGDTNKVRSQAIAMDSAYLTSQNEPFYHDDSTIAMRKGPKGSEVLTVLTNSGSKGSYTVSIDAGYGSGTELVDLYSCNTATVGSDGTVSVKISAGMPQALVPASAAGNSALCGNSSK

SEQ ID NO: 41 - protein, 609 aa, Talaromyces amestolkiae
MKFSNVSLVWSALASVALGLTAAEWRSQSIYFLLTDRFALTSNSTTASCDVADGLYCGGSWQGVINHLDYIQGMGFTAIWITPVTENFEGDTSDGEAYHGYWQQNAYATNSHYGASSDLLKLSEALHARGMYLMVDIVVNNMAYDGAGTSVDYSIFNPFPSKSYYHSYCLIDYSTYNATDWNVCWEGDNIVSLPDIDTTQTYVKDTWGTWVESFVANYSIDGLRIDSAGHIQQDFFTAFEESSGVYCIGEVDYGDPAVVCPYQNYLSGVLNYPMYYQLLYAFESSSGSISNLYNMINTVKSDCADTSLLGNFIENHDNPRFAYYTSDYSEAKNVISFIFLTDGIPILYYGQEQHYNGGNIPLNREALWTSDYSTTAQLYTHTKTSNAIRSLAITKDSAFLTYKNTPIYQDSNTIAMRKGTTGLQLVTVLSNLGASGSSSTLTLSGSGYTSGTVVTELYTCTNVTVSSSGTIAVPMASGSPRAFLPWSSVSGSSLCSGSGSSCTAASTVAVTFEEVVTTTYGQEVYISGSISQLGNWSTSSAVLLSASQYTSSDPVWTVTISLPAGESFQYKFIIVNTDGSITWESDPNRSYTVPTGCAGLTATVDDTWR

SEQ ID NO: 42 - protein, 497 aa, Talaromyces rugulosus
MKLSLLGGTTAVALLSGVANAATPDDWRSQSIYFLLTDRFARADNSTTATCNTEDGIYCGGSWQGVINQLDYIQGMGFTAIWISPVTGQLTEVTSDGSSYHGYWQQDIYALNENFGTSDDLNDLASALHDRGMYLMVDVVANHMGYAGDPTTVDYSVFNPFNSQDYFHPYCALTDYTNQTMTEECWMGDDTVCLPDLDTESTSVQDIWFSWIGDLVANYSIDGIRIDTVAEVSKDFWATYNEKAGVYAVGEVDNGDVTYACPYQTALDGILNYPTYFPLVRAFESSSGSISELADMINSVKSDCTDTNLLGSFSENHDNPRFASYTQDLALAKNVLTYTILADGIPIIYAGQEQHYDGAEDPANREATWLSGYNTTSELYTWVAKMNKIRSFAIKSDDGYTTYNNYPIYQDDNNLGMRKGSNGSQIITVVSNLGADGGQTTLTVSGGGYTAGTVLTELITCTSVTVGDGGDISVSLDSGLPSVLYPASKLNGTGSPC

SEQ ID NO: 43 - protein, 493 aa, Thermoascus crustaceus
MHLRSFSILPALVVGTALAATPAQWRSQSVYFLLTDRFARTDGSTTAACDTDARAYCGGTWQGIIDHLDYIQGMGFTAIWITPVTENLPQDTGDGTSYHGYWQQDVYSLNSNYGTPDDLRALSSALHDRGMYLMVDVVANHMGYAGPGSSVDYTVFTPFNDQKYFHPYCSISNYDDQSNVEDCWLGDSTVSLPDLDTTRSDVQDMWYSWVKGLVANYSVDGLRIDTVKHVQKDFWPGYNDAAGVYCVGEVFDGDPSSTCDYQNYLDGVLNYPMYYPLLRAFSSTSGSISDLYNMINTVKSECADSTLLGTFVENHDNPRFASYTSDISLAKNALAFTILSDGIPIIYAGQEQHYSGGNDPANREAVWLSGYSTTSELYKFIAVSNQIRNHAISVDGDDYLTYKTYPIYQDTTTLAVRKGSLITVLSNLGSSGSSYTLSLGGTGYSSGQELMEIYSCTTVTADSSGNIAVPMGSGLPKAFYPTANLGGSGICGK

SEQ ID NO: 44 - protein, 490 aa, Aspergillus puulaauensis
MRSTLLLSLAATALAATPAQWRSQSIYFLLTDRFARTDNSTTAECDTSAGRYCGGSWQGIINQLDYIQGMGFTAIWITPVTAQVEDSSSGDAYHGYWQQDLYFLNSQLGTKDDLLALSDALHERDMYLMVDVVANHMGYDGAADSVDYSVFNPFNSQDYFHSPCSIDDYDDQTQVEECWLSTSAVSLPDVDTTRDDVKTLWYDWVEALVSNYSIDGLRVDTVRHVQKDFWADFNDAAGVYCVGEVLQGDPEYTCAYQELMDGVLNYPIYYPLLRAFSSTSGSISELYDMINSVKSTCADSTLLGSFIENHDNPRFASETDDISLAKNVAAFVILSDGIPIIYAGQEQHYAGGEDPENREATWLSGFDTSSELYKLIAASNAIRTHAIGQDDEWITYKNYPIYQDTSSLAMRKGNNGTQVVTVLTNAGAGGNSYTLSLPDTGYSAGAALTEVLSCTDITVSDNGEVPVPMESGLPRVLYPTAKLEGSGICQ

SEQ ID NO: 45 - protein, 490 aa, Aspergillus nidulans
MRSPLFLSFAATVLAATPAEWRSQSIYFLLTDRFARTDNSTTAECDTSAKYCGGTWQGIINQLDYIQGMGFTAIWITPVTANLEDGQHGEAYHGYWQQDIYALNPHFGTQDDLRALSDALHDRGMYLMVDVVANHFGYDAPAASVDYSAFNPFNSADYFHTPCDITDYDNQTQVEDCWLYTDAVSLPDVDTTNEEVKEIWYDWVGDLVSDYSIDGLRIDTARHVQKDFWRDYNDAAGVYCVGEVFQGDPDYTCGYQEVMDGVLNYPIYYPLLRAFSSTSGSLSDLANMIETVKYTCSDATLLGNFIENHDNPRFASYTDDISLAKNVAAFVILSDGIPIIYAGQEQHYSGAGDPANREATWLSGYDSTSELYQFISKTNQIRNHAIWQNETYLSYKNYAIYNENNVLAMRKGFDGSQIITILTNAGADAGSSTVSVPNTGFTAGAAVTEIYTCEDITVSGSGEVSVPMESGLPRVLYPKAKLEGSGICGL

SEQ ID NO: 46 - protein, 503 aa, Talaromyces atroroseus
MKFSLAAIAAIASLLAPVVKAATPDDWRSRSIYFLLTDRFARTDNSTTATCETLYGVCSKTTVEDHGRLDYIQDMGFTAIWISPVTEQLPQSTADGEAYHGYWQQDIYALNSNFGTAEDLQNLATALHDRGMYLMVDVVANHFGYAGAGDDVDYAIFNPFNSESYFHPFCLITDYSNETMVEECWEGDNIVSLPDLDTESTAVQNICLTRSYDNEDEATANGNYEPVDGLRLDSVMEVQKDFWPDWNSASGVYCVGEVDDGDPTFTCPYQKYLDGVLNYPTYFPLVRAFESSSGSISELYDMINEVKSDCVDSNLLGSFSENHDNPRFASYTSDFSLAKNALAFTILSDGIPIIYAGQEQHYDGGNNPYNREATWLSGYDTSAELYTFIAVLNQIRNYAIQWDNGYTTYKTYPIYQDDNNLAIRKGSDESQIITVLSNVGTDADSYSLTISGTGYTAGEVLTDLISCTNVTVDDSGDVSVTMTGGLPSVLYPTYKLVYDGHPC

SEQ ID NO: 47 - protein, 501 aa, Penicillium italicum
MILAQFACLAGLIGTAIAATPAEWRSRSIYFMLTDRFARTDGSTTAACNTGDRKYCGGTWQGIIDKLDYIQGMGFTAIWITPVTSQLTVDTPYGEPYHGYWQQDIYALNSNYGTADDLKALAAALHERDMYLMVDVVANHMGYNGAGADVDYTKFNPFNDAKYFHPYCPITDYDNDTMAQNCWLGDDKVSLPDLDTQSMEVQDIWYDWVRSLVSNYSKTDEYKVDGLRIDTVKHVQKDFWPGYNKAAGVYCVGEVFDGDVDYTCPYQEVMDGVLNYPVYYPLLKAFQSTSGSMADLFNMINRVKSTCKDSTLLGNFLENHDNPRFASVTDDIALAKNAATFTIMADGIPIVYAGQEQHYSGGEDPANREALWLSGFNTDSELYKLIATVNGARNQAIAKSTNYTIYQNYPIYKDSSTIAMRKGYDSGQTITVLTNLGAGGKDYSVSIPDTGFAAGAKLTEVISCVSVTAGESGEVSVPMAGGAPRILLPTSLLEGSTLCSS

SEQ ID NO: 48 - protein, 616 aa, Aspergillus welwitschiae
LSAAEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISDLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGSNSSTTTTTTATSSSTATSKSASTSSTSTACTATSTSLAITFEELVTTTYGEEIYLSGSISQLGDWDTSDAVKMSADDYTSSNPEWSVTVTLPVGTTFEYKFIKVESDGSVTWESDPNREYTVPECGSGETVVDTWR

SEQ ID NO: 49 - protein, 615 aa, Aspergillus brasiliensis
LSAAEWRTQSIYFLLTDRFGRTDNSTTATCNTGDQIYCGGSWQGIINHLDYIQGMGFTAIWISPITEQLPQDTSDGEAYHGYWQQKIYDVNSNFGTADDLKSLSDALHARGMYLMIDVVPNHMGYAGSGNDVDYSVFDPFDSSSYFHPYCLITDWDNLTMVQDCWEGDTIVSLPDLNTTETVVRTIWYDWVADLVSNYSVDGLRIDSVLEVEPDFFPGYQEAAGVYCVGEVDNGNPALDCPYQDYLDGVLNYPIYWQLLYAFESSSGSISDLYNMIKSVASDCSDPTLLGNFIENHDNPRFAYYTSDYSQAKNVLSYIFLSDGIPIVYAGEEQHYSGGDVPYNREATWLSGYDTSAELYTWIATTNAIRKLAIAADSSYITYANDPIYTDSNTIAMRKGTSGSQVITVLSNKGSSGSSYTLTLSGSGYTSGTKLIEAYTCTSVTVDSNGDIPVPMASGLPRVLLPASVVDDSSLCGGSGSSTSTTTSTATATTTSKTSTTSSSSSSSSCTASATAIPITFEELVTTTYGEEIYLSGSISQLGDWDTSDAVKLSADDYTSSNPEWSVTVTLPVGTTFEYKFIKVESGGSVTWESDPNREYTVPECGSGETVVDTWR

SEQ ID NO: 50 - protein, 609 aa, Aspergillus sclerotiicarbonarius
LSAAEWRSQSIYFLLTDRFGRTDNSTTATCDTGEQIYCGGSWQGVINHLDYIQGMGFTAIWISPVTEQLPQDTADGEAYHGYWQQEIYTVNSNFGTADDLKALSDALHARGMYLMVDVVPNHMGYAGDGDDVDYSVFDPFDSSSYFHPYCLITDWSNLTMVRDCWEGDTIVSLPDLNTTETVVRTIWYDWVADLVANYSVDGLRIDSALEVEPDFFPGYQEAAGVYCVGEVDNGDPTLDCPYQEYLDGVLNYPIYWQLLYAFESSSGSISDLYNMIKSVASDCSDPTLLGNFIENHDNPRFAYYTSDYSEAKNVLSYIFLSDGIPIVYAGEEQHYNGGDVPYNREATWLSGYDTSAELYTWIATTNAIRKLAIEADSDYITYANDPFYTDSNTIAMRKGTSSQQVITVLSNKGSSGSSYTLTLSGSGYTSGTELIEAYTCTSVTVDSNGDIPVPMDSGLPRVFLPASSINSSSSSLCGGSSTTTTTTTSTSPSTATSSSTATATTTCTTLPITFIESVTTTYGEEIYLSGSITQLGDWDTSDAVLLSADNYTSSNPEWYVEVSLPTGTSFEYKFIKKESDGSVTWESDPNREYTVPEECGGVTVDDSWR

SEQ ID NO: 51 - protein, 607 aa, Talaromyces rugulosus
LTPAEWRSQSIYFLLTDRFGREDNSTTASCDLSERIYCGGSWQGIINHLDYIQGMGFTAIWITPVTEQLPQDTGEGEAYHGYWQQEIYSVNSNYGTAADLLALSEALHDRGMYLMVDVVANHMGYAGAGDSVDYSVFSPFDSSSYFHSYCLISDYSNQENVEDCWLGDTTVSLPDLNTDLTSVQTLWYDWVADLVSNYSIDGLRIDTVKHVQESFWPGYNSAAGVYCVGEVFDGDPAYTCPYQNYLDAVLNYPIYYQLLYAFESSSGSISGLYDMISSVASDCADPTLLGNFIENHDNPRFASYTDDYSQAKNVISFVFLSDGIPIIYAGQEQHYSGGSDPENREATWLSGYSTTAELYQFIATTNKIRALAVSSDSSYITTKNDPFYTDSNTIAMQKGSSGSQVVTVLSNMGASGSSYTLTLSGSGYASGTELMEMYTCTSVTVDSSGNIAVPMASGLPRVFMLASSAGSLCGSSTTTTTSKITSTTSTSSTSSTSTSCTQTTDVPVLFKELVTTTYGEDIYISGSISELGDWDTANAVALSSSGYTSSNPLWQVVVSIPAGTSFEYKFFETGSSSSVTWESDPNRSYTVPTACSGSTATVTATWR

SEQ ID NO: 52 - protein, 617 aa, Penicillium rolfsii
LTPAEWRSQSIYFLLTDRFGRTDNSTTATCNVSDRIYCGGSWQGIINHLDYIQGMGFTAIWITPVTEQLSQDTGDGEAYHGYWQQEIYNVNTNYGTAADLLALSKALHSRGMYLMVDVVANHMGYDGAGNTVDYSVFNPFDSSSYFHSYCEITDYSNQTNVEDCWLGDTTVSLPDLDTTLSSVQTIWYNWVTGLVSNYSIDGLRIDTVKHVQKSFWPGYNSAAGVYCMGEVFDGDPAYTCAYQSYLDGILNYPVYYQLLYAFESTSGSISNLYNMINSVASDCSDPTLLGNFIENHDNPRFASYTSDYSQAKNVISFIFFSDGIPIVYAGQEQHYSGGSDPANREATWLSGYDKTAQLYTYITTTNKIRALAISKDSAYITSKNTAFYSDSNTIAMKKGSSGSQVITVLSNRGSSGSSYTLSLSGSGYSSGKKLMEMYTCTAVTVDSSGNIAVPMASGLPRVYMLASSACSICSSTCSTTTSTASSTSSSTTTASTTLKTTASSASTSCTQATALPVLFKEIVTTSYGQNIYISGSISQLGSWDTSSAVALSADQYTSSNHLWYVVVTIPVGTSFQYKFIEETSGYSTITWESDPNRSYTVPTGCVGSTATVTATWR

SEQ ID NO: 53 - protein, 596 aa, Aspergillus felis
LTPAEWRSQSIYFLLTDRFGRQDNSTTAACDVTQRLDYIQGMGFTAVWITPVTEQFYEDTGDGTSYHGYWQQNIYEVNSNYGTAQDLKSLANALHARGMYLMVDVVANHMGYDGAGTSVDYSVFNPFNSSSYFHPYCLISDYSNQTNVEDCWLGDTTVSLPDLNTTETTVRTIWYDWVKALVANYSIDGLRIDTVKHVEKAFWPDYNSAAGVYCVGEVFSGDPAYTCPYQNYLDGVLNYPIYYQLLYAFESTSGSISNLYNMISSVASDCADPTLLGNFIENHDNPRFASYTSDYSQAKNVVSFIFFSDGIPIVYAGQEQHYSGGADPANREAVWLSGYSTSATLYSWIASTNKIRKLAISKDSAYVTSKNNPFYYDPNTLAMRKGSTAGAQVITVLSNKGSSGSSYTLSLSGTGYSAGATLVEMYTCTTLTVDSSGNLAVPMASGLPRVLVPASWVSGSGLCGGSVSTTATTTTSATTTTTATTTTACTSATALPILFEELVTTTYGENIYLTGSISQLSNWDTSSAIALSAGNYTSSNPEWYVTVTLPIGTSFQYKFFKKESDGSITWESDPNRSYTVPTGCAGTTVTVSDTWR

SEQ ID NO: 54 - protein, 591 aa, Aspergillus hiratsukae
LTPAEWRTQSIYFLLTDRFGREDNSTTAACDVTERLDYIQGMGFTAIWINPVTEQFYEDTGDGTSYHGYWQQNIYEANHNYGTAQDLKSLADALHARGMYLMVDVVANHMGYDGAGNSIDYSVFTPFDSSSYFHPYCLISDYSNQTNVEDCWLGDTTVSLPDLDTTDAKVRTIWYDWVKGLVANYSIDGLRIDTVKHVEKDFWPGYNDAAGVYCVGEVFSGDPTYTCPYQNYLDGVLNYPIYYELLYAFESPRGSISNLYNMISSVASDCADPTLLGNFIENHDNPRFASYTSDYSQAKNVISFMFFSDGIPIVYAGQEQHYSGGADPANREAVWLSGYSTSATLYSWIASTNRIRKLAISKDTAYITSKNNPFYYDSNTLAMRKGSVAGAQVVTVLSNKGSSGSSYTLSLSGTGYSAGASLVEMYTCTTLTVDSSGNLAVPMASGLPRVLVPSSWVSGSGLCGSGSTTTTTTTTTATATTTACTSATALPILFEEILTTTFGENIYLSGSISQLGNWDTSSAIALSASKYTSSNPEWYVTVTVPVGTSFQYKFFKNVSDGSIVWETDPNRSYTVPSGCGGTTVTLSGTWR

SEQ ID NO: 55 - protein, 595 aa, Aspergillus viridinutans
LTPAEWRSQSIYFLLTDRFGREDNSTTAACDVTQRLDYIQGMGFTAIWITPVTEQFYEDTGDGTSYHGYWQQNIYEVNSNYGTAQDLKSLADALHARGMYLMVDVVANHMGYDGAGNSVDYSVFTPFGSSSYFHPYCLISDYSNQTNVEDCWLGDTTVSLPDLDTTDTTVQTICTRTWAWMILTVIVDGLRIDTVKHVEKDFWPGYNDAAGVYCVGEVFSGDPTYTCPYQNYLDGVLNYPIYYQLLYAFESTSGRISNLYNMLSSVASDCADPTLLGNFIENHDNPRFASYTSDYSQAKNVISFMFFSDGIPIVYAGQEQHYSGGADPANREAVWLSGYSTSATLYSWIASTNKIRKLAISKDSAYITSKNNPFYYDSNTLAMRKGSTAGSQVITVLSNKGFSGSSYTLSLSGTGYSAGTTLVEMYTCTSLTVDLSGNLAVPMASGLPLVLVPSSWVSGSGLCGSSVSTTATATSTTKTTIATTTAACTSATALPILFEELVTTTYGENIYLTGSISQLGNWDTSSAIALSASKYTSSNPEWYVTVTLPVGTSLQYKFFKKESDGSIVWESDPNRSYTVPTGCAGTTVTASDTWR

SEQ ID NO: 56 - protein, 483 aa, Aspergillus sclerotialis
LSPAEWRSQSIYFLLTDRFGRTDNSTTAECDVEKRLYCGGTWQGIINQLDYIQGMGFTAIWITPVTEQLSQDTGYGEAYHGYWQQKIYNLDSHLGTADDLKALSKALHDRGMYLMVDVVANHMGYDGPGNSVDYSVFDPFDSSSYFHKYCLISNYDDQNDVQNCWLGDTTVSLPDLNTKDAEVRELWYDWVTGLVSNYSIDGLRIDTVKHVEKTFWEGYNQAAGVYCVGEVFDGDPKYTCAYQSYLDAVLNYPIYYPLLNAFKAPGGNMGDLYNMVNSVASHCSDPTVLGNFIENHDNPRFASYTDDYSLAKNAITFMFLSDGIPIIYAGEEQHYSGGSDPGNREAVWLSGYSTDAELYKFIATTNKIRSLVILKDNKYVTSKNVPFYQDDNTFAMRKGSGNSQAITVLSNAGSSSDSYSVTVKGAEYSSGTQLIDLYSCAKVTVGDDGSISLQVESGLPKVLVPASYAGDALCGGSSAPSIA

SEQ ID NO: 57 - protein, 610 aa, Penicillium steckii
LSPAEWRSQSIYFLLTDRFGREDNSTTASCDVSNRLDYIQDMGFTAIWITPVTEQLQGDTGYGEAYHGYWQQDIYDVNENYGTAEDLLALSNALHDRGMYLMVDVVANHMGYNGAGNTVDYSVFNPFDSSSYFHSYCLITNYDNQDNVEDCWLGDTTVSLPDLNTDLSSVQTIWYDWISDLVSNYSIDGLRIDTVKHVQKSFWPSYNSAAGVYCVGEIFDGNPAYTCDYQNYMDGVLNYPIYYQLLYAFQSTSGSISDLYNMIGTVASDCKDPTLLGNFIENHDLPRFASFTSDYSLAKNVLSFLFFSDGIPIVYAGQEQHYSGKDDPANREATWLSGYSTSAELYKHISTTNKIRKLAASSDSSYITSRVIIVSSKLDKPKGLTNSMQNTPFYQDSHTIAMKKGSSGSQVITVLSNYGSSGSSYTLTLGSSGYSSGTELMEMYTCTTATVDSSGNIPVPMVSGLPRVLMLASVAKSSGPCASSGSPTTSTASSSSTTMSACTKATALPVLFKETVSTSYGEDIYISGSISRLGSWDASNAVALSADQYTSSNPLWYVSITLPVGTSFEYKFIKKTSGSSTVTWESDPNRSYTVPTACSGSTATVTATWR

SEQ ID NO: 58 - protein, 478 aa, Aspergillus leporis
ATPAEWRSQSIYFLLTDRFARTDGSTTASCNTEDRKYCGGTWQGIIDKLDYIQGMGFTAIWITPVTGQLPQHTAYGDAYHGYWQQDIYALNSNYGTADDLKALASALHDRGMYLMVDVVANHMGYDGAGDSVDYSVFKPFNSKDYFHNFCLIQNYDDQTQSENCWLGDNSVSLPDLDTTREDVKGLWYDWVEALVSNYSIDGLRIDTVKHVQKDFWPGYNQAAGVYCIGEVLNGDPAYTCPYQNVMDGVLNYPMYYPLLNAFKSTSGSISDLYNMINTVKSTCPDSTLLGTFVENHDNPRFASYTNDMSLAKNVAAFIILADGLPIIYAGQEQHYAGGNDPANREATWFSGYSTTSEIYKLIASSNAIRNHAISTDSGYLTYKNWPVYQDASTIAMRKGTDGSQIITVLSNQGSSGGSYTLSLTGTGYTAGQQLTEITTCSTVTVGLDGKVPLPMEKGLPKVLYPSAKLSGSKVCSSS

SEQ ID NO: 59 - protein, 477 aa, Aspergillus tanneri
ATPADWRSQSIYFLLTDRFARSDGSTTASCNTEDRKYCGGTWKGIIDQLDYIQGMGFTAIWITPVTGQLPQDTGYGEAYHGYWQQDIYSLNDKYGTADDLKALADALHNRHMYLMVDVVANHMGYSGAGDSVDYSVFKPFNSKDYFHPYCPIKDYNDQTQAENCWLGDNTVSLPDLDTTKSEVKDLWYKWVGDLVSNYSIDGLRIDTVKHVQKDFWPGYNKAAGVYCVGEVLNGDTAFTCAYQSVLDGVLNYPMYYPLLNAFKSTSGNIGDLYNMINNVKSNCPDSTLLGTFIENHDNPRFASYTNDMSLAKNVAVFTILADGIPIIYAGQEQHYAGGEDPANREATWLSGYSTKSELYTLIASSNAIRNHAIAQDPKYVTYKNYPIYQDETTIAMRKGSDGSQVVTVLSNKGSSGSSYTLSLGDTGYSSGQNVTEIYTCETLTVGSDGKVSVPMNNGLPRVLYPSEKLKGSSLCSE

SEQ ID NO: 60 - protein, 476 aa, Aspergillus avenaceus
ATPADWRSQSIYFLLTDRFARTDGSTSATCNTEDRKYCGGTWKGIIDQLDYIQGMGFTAIWITPVTSQLTQDTAYGEAYHGYWQQDIYSLDSHLGTADDLRALASALHERGMYLMVDVVANHMGYDGAGDSVDYSVFNPFNSKDYFHPFCLIQDYNDQTQSENCWLGDNSVSLPDLDTTTQDVKNIWYDWVEELVSNYSIDGLRVDTVKHVQKDFWPDYNKAAGVYCIGEVLNGDPSYTCPYQEVMDGVLNYPIYYPLLNAFKSTSGNMNDLYNMINTVKSDCPDSTLLGTFIENHDSPRFASYTNDMALAKNVAAFIILADGIPIIYAGQEQHYAGGNDPSNREATWLSGYSTTSDLYKLIASSNAIRNHAVSTDKGYVTYKNWPIYKDTTTIAMRKGTDGSQIITILSNKGASGDGYTLSLDGTGYDAGTELTEILSCSTLTVGSDGKVPVPMEKGLPRVLYPSAKLGDSKICT

SEQ ID NO: 61 - protein, 477 aa, Aspergillus novofumigatus
ATPEQWRSQSIYFLLTDRFARTDGSTTASCDTTARKYCGGTWQGIIKQLDYIQGMGFTAIWITPVTEQLPQDTSEGTAYHGYWQQDIYSVNSNYGTADDLKALGSALHDRGMYLMVDVVANHMGYAGAGNSVDYSLFSPFNSQTYFHPLCFISNYDNQTNVENCWLGDNVVPLADLDTTKSDVQKIWYNWVGSLVSNYSIDGLRIDTVKHVQKDFWPGFNDAAGVYCIGEVFDGDPAYTCPYQDVLDGVLNYPTYYPLLKAFQSTSGSMSDLYNMINTVKSQCADSTLLGTFVENHDTPGSHRKYTNDMALAKSAAAFIILSDGIPIIYAGQEQHYSGGTDPANREAVWLSGYSKTSELYKLIASANAIRNHAISKDPEYVTYKNNPIYKDTSTIAMRKGADGAQVITVLSNLGASGSSYTLSLSGTGYDVGQQLTEVFSCATVTVDSDGKVPVAMASGLPRAFYPTAGLNGSTICA

SEQ ID NO: 62 - protein, 478 aa, Aspergillus nomiae
ATPADWRSQSIYFLLTDRFARTDGSTTAACNTEDRKYCGGTWQGIIDKLDYIQGMGFTAIWITPVTGQLPQNTAYGEAYHGYWQQDIYSLNENYGTPDDLKALASALHTRGMYLMVDVVANHMGYDGAGASVDYSVFKPFNSQEYFHPFCLIQNYNDQTQSEDCWLGDNSVSLPDLDTTKDEVKNEWYEWVGSLVSNYSIDGLRVDTVKHVQKDFWPGYNKAAGVYCIGEVLNGDPAYTCPYQDVMDGVLNYPIYYPLLNAFKSTSGSMDDLYNMINTVKSDCPDSTLLGTFVENHDNPRFASYTNDIALAKNVAAFIILNDGIPIIYAGQEQHFAGGNDPANREATWLSGFSTDSEIYKLIASANAIRSHAISIDTGFVTYKNWPIYKDTTTIAMRKGTDGSQVVTILSNKGASGDSYTLSLGNTGYTAGQKLTEVIGCTTVTVGSDGTVPVPMAGGLPRILYPTEKLGDSKICSSA

SEQ ID NO: 63 - protein, 478 aa, Aspergillus flavus
ATPAEWRSQSIYFLLTDRFARTDGSTTASCNTEDRKYCGGTWQGIIDKLDYIQGMGFTAIWITPVTGQLPQHTAYGDAYHGYWQQDIYTLNRNYGTADDLKALASALHDRGMYLMIDVVVNHMGYDGAGDSVNYSVFKPFNTKDYFHNFCFIQNFDDQTQSENCWLGDNRVSLPDLDTTREDVKGLWYDWVEAFVSNYSIDGLRVDTVKHVQKDFWRGYNEAAGVYCIGEVFNGDPAYTCPYQNIMDGVLNYPMYYPLLNAFKSTSGSISNLYNMINTVKSTCPDSTLLGTFVENHDNPRFASYTNDMSLAKNVAAFIILADGLPIIYAGQEQHYAGGNDPANRDATWFSGYSTTSELYKLIASLNAIRRHAINTDSGYLSYKNWPIYQDASTIAMRKGTDGSQIITVLSNQGSSGGSYTLSLTGTGYTAGEQLHEIINCSTVTVGSDGKVLLPMEKGLPRVLYPSAKLNGSKVCSSS

SEQ ID NO: 64 - protein, 603 aa, Aspergillus udagawae
LNAAEWRSQCIYFLMTDRFARTDGSTTAPCDVGQRGYCGGSWKGIIDHLAYIQGMGFTAIWITPIVEQIPQTTTEGTGFHGYWPQNIYSVNSHFGTADDIRALSKALHDRGMYLMMDGVANHMGFNGPGASNGFSTFTPFNSASYFHSYCPINNYNDQWQVENCYLRDSVVSLTDLNTQSSAVRGIWYDWVKDLVANYSVDGLRIDTAKHVEKDFWSGYTQAAGVYSVGEILHGDPAYTCPYQGYMDGVMNYPIYYQLVNAFKSSSGSISSLSNMISSVASKCKDPTLLGNFIENHDNPRFPSYTSDISQAKSVIAYVFLTDGIPIIYSGQEQHLSGGADPYNREALWLSGYSTNSELYKFIAMTNKIRRLAISKDLNYLPARNNPFYTDSNTIAMKKGSGGSNVITVLTNSGSNAGSYTLNLNSHGYSSGSSLIELYTCSSVQVDSNGNLPVPMSSGLPRVLVPSAWVPGSGLCGASSTTSLATPTVTSPSPGTCAPSTAIPVIFKERVATSYGENIFLSGSIDQLGNWDTSKAVALSASGYTSSNPVWSVKLDLHAGTYFQYKFIKKGQDGSVMWESDPNRSYTLPSGCVETAISIADSWR

SEQ ID NO: 65 - protein, 509 aa, Aspergillus ellipticus
ATPADWRSQSIYFLLTDRFARTDNSTTASCNTGARQYCGGTWQGIINQLDYIQGMGFTAIWITPVTGQIPQETGYGEAYHGYWQQDAYALNSHYGTADDLKKLVSALHARGMYLMVDVVANHMAYNGSADTTDYSAYNPFNSKEYFHKICSIEDNNNQTQTEDCWLADSIVSLPDLNTTQTDVKNMWYDWIGSLVSNYSIDGLRIDTVKNVQQNFWPDYNTAAGVYCVGEVFDGDAEYTCPYQNYLDGVLNYPMYYPILRAFQSTSGSISDLYNMINTVKSTCKDSTLLGTFVENHDNPRFANYTSDMSLAKNAAAFTMLADGIPIIYAGQEQHYHGGNDPYNREATWLAKYSTTSPLYELIATSNRIRSHAISQDSGYITYKNYPIYQDNSTLAMRKGYNGSQTITVLSNLGASGKTYNLSLPGTGYTANQQLMEIYTCTNITVDSSGSVSVPMASGLPRVLYPADKLNGSLLCNSTTSSATSLSPLSNSAIPIYLTLAAFVVQLSQL

SEQ ID NO: 66 - protein, 478 aa, Aspergillus sp.
LSPAEWRGQSIYFLLTDRFARADNSTTAPCDVTDRVYCGGSWQGIINQLDYIQGMGFTAIWVSPVTEQLPQDTEYGEAYHGYWQQDIYNLNKHFGTPDDLNALADALHERGMYLMVDVVPNHMGYDGPGDSVDYGVFEPFSSSSYFHPFCFIENYDNQDNVEQCWLGDSKVPLPDLDTSREDVQNIWYDWVSGLVSNYSVDGLRLDTAKHVQKNFWPDYNKAAGVYCVGEVLSGDPKYTCPYQEYMDAVLNYPIYYQLLDAFKSTNGPTDKVYNMISRVASSCRDPTLLGNFIENHDAPRFASYTQDLALAKNAIAFLFLTDGIPIVYAGQEQHYSGGDDPFNRGAIWLSGYSTEAALYRFIGDTNKVRSQAIAMDSAYLTSQNEPFYHDDSTIAMRKGPKGSEVLTVLTNSGSKGSYTVSIDAGYGSGTELVDLYSCNTATVGSDGTVSVKISAGMPQALVPASAAGNSALCGNSSK

SEQ ID NO: 67 - protein, 590 aa, Talaromyces amestolkiae
LTAAEWRSQSIYFLLTDRFALTSNSTTASCDVADGLYCGGSWQGVINHLDYIQGMGFTAIWITPVTENFEGDTSDGEAYHGYWQQNAYATNSHYGASSDLLKLSEALHARGMYLMVDIVVNNMAYDGAGTSVDYSIFNPFPSKSYYHSYCLIDYSTYNATDWNVCWEGDNIVSLPDIDTTQTYVKDTWGTWVESFVANYSIDGLRIDSAGHIQQDFFTAFEESSGVYCIGEVDYGDPAVVCPYQNYLSGVLNYPMYYQLLYAFESSSGSISNLYNMINTVKSDCADTSLLGNFIENHDNPRFAYYTSDYSEAKNVISFIFLTDGIPILYYGQEQHYNGGNIPLNREALWTSDYSTTAQLYTHTKTSNAIRSLAITKDSAFLTYKNTPIYQDSNTIAMRKGTTGLQLVTVLSNLGASGSSSTLTLSGSGYTSGTVVTELYTCTNVTVSSSGTIAVPMASGSPRAFLPWSSVSGSSLCSGSGSSCTAASTVAVTFEEVVTTTYGQEVYISGSISQLGNWSTSSAVLLSASQYTSSDPVWTVTISLPAGESFQYKFIIVNTDGSITWESDPNRSYTVPTGCAGLTATVDDTWR

SEQ ID NO: 68 - protein, 476 aa, Talaromyces rugulosus
ATPDDWRSQSIYFLLTDRFARADNSTTATCNTEDGIYCGGSWQGVINQLDYIQGMGFTAIWISPVTGQLTEVTSDGSSYHGYWQQDIYALNENFGTSDDLNDLASALHDRGMYLMVDVVANHMGYAGDPTTVDYSVFNPFNSQDYFHPYCALTDYTNQTMTEECWMGDDTVCLPDLDTESTSVQDIWFSWIGDLVANYSIDGIRIDTVAEVSKDFWATYNEKAGVYAVGEVDNGDVTYACPYQTALDGILNYPTYFPLVRAFESSSGSISELADMINSVKSDCTDTNLLGSFSENHDNPRFASYTQDLALAKNVLTYTILADGIPIIYAGQEQHYDGAEDPANREATWLSGYNTTSELYTWVAKMNKIRSFAIKSDDGYTTYNNYPIYQDDNNLGMRKGSNGSQIITVVSNLGADGGQTTLTVSGGGYTAGTVLTELITCTSVTVGDGGDISVSLDSGLPSVLYPASKLNGTGSPC

SEQ ID NO: 69 - protein, 474 aa, Thermoascus crustaceus
ATPAQWRSQSVYFLLTDRFARTDGSTTAACDTDARAYCGGTWQGIIDHLDYIQGMGFTAIWITPVTENLPQDTGDGTSYHGYWQQDVYSLNSNYGTPDDLRALSSALHDRGMYLMVDVVANHMGYAGPGSSVDYTVFTPFNDQKYFHPYCSISNYDDQSNVEDCWLGDSTVSLPDLDTTRSDVQDMWYSWVKGLVANYSVDGLRIDTVKHVQKDFWPGYNDAAGVYCVGEVFDGDPSSTCDYQNYLDGVLNYPMYYPLLRAFSSTSGSISDLYNMINTVKSECADSTLLGTFVENHDNPRFASYTSDISLAKNALAFTILSDGIPIIYAGQEQHYSGGNDPANREAVWLSGYSTTSELYKFIAVSNQIRNHAISVDGDDYLTYKTYPIYQDTTTLAVRKGSLITVLSNLGSSGSSYTLSLGGTGYSSGQELMEIYSCTTVTADSSGNIAVPMGSGLPKAFYPTANLGGSGICGK

SEQ ID NO: 70 - protein, 475 aa, Aspergillus puulaauensis
ATPAQWRSQSIYFLLTDRFARTDNSTTAECDTSAGRYCGGSWQGIINQLDYIQGMGFTAIWITPVTAQVEDSSSGDAYHGYWQQDLYFLNSQLGTKDDLLALSDALHERDMYLMVDVVANHMGYDGAADSVDYSVFNPFNSQDYFHSPCSIDDYDDQTQVEECWLSTSAVSLPDVDTTRDDVKTLWYDWVEALVSNYSIDGLRVDTVRHVQKDFWADFNDAAGVYCVGEVLQGDPEYTCAYQELMDGVLNYPIYYPLLRAFSSTSGSISELYDMINSVKSTCADSTLLGSFIENHDNPRFASETDDISLAKNVAAFVILSDGIPIIYAGQEQHYAGGEDPENREATWLSGFDTSSELYKLIAASNAIRTHAIGQDDEWITYKNYPIYQDTSSLAMRKGNNGTQVVTVLTNAGAGGNSYTLSLPDTGYSAGAALTEVLSCTDITVSDNGEVPVPMESGLPRVLYPTAKLEGSGICQ

SEQ ID NO: 71 - protein, 475 aa, Aspergillus nidulans
ATPAEWRSQSIYFLLTDRFARTDNSTTAECDTSAKYCGGTWQGIINQLDYIQGMGFTAIWITPVTANLEDGQHGEAYHGYWQQDIYALNPHFGTQDDLRALSDALHDRGMYLMVDVVANHFGYDAPAASVDYSAFNPFNSADYFHTPCDITDYDNQTQVEDCWLYTDAVSLPDVDTTNEEVKEIWYDWVGDLVSDYSIDGLRIDTARHVQKDFWRDYNDAAGVYCVGEVFQGDPDYTCGYQEVMDGVLNYPIYYPLLRAFSSTSGSLSDLANMIETVKYTCSDATLLGNFIENHDNPRFASYTDDISLAKNVAAFVILSDGIPIIYAGQEQHYSGAGDPANREATWLSGYDSTSELYQFISKTNQIRNHAIWQNETYLSYKNYAIYNENNVLAMRKGFDGSQIITILTNAGADAGSSTVSVPNTGFTAGAAVTEIYTCEDITVSGSGEVSVPMESGLPRVLYPKAKLEGSGICGL

SEQ ID NO: 72 - protein, 482 aa, Talaromyces atroroseus
ATPDDWRSRSIYFLLTDRFARTDNSTTATCETLYGVCSKTTVEDHGRLDYIQDMGFTAIWISPVTEQLPQSTADGEAYHGYWQQDIYALNSNFGTAEDLQNLATALHDRGMYLMVDVVANHFGYAGAGDDVDYAIFNPFNSESYFHPFCLITDYSNETMVEECWEGDNIVSLPDLDTESTAVQNICLTRSYDNEDEATANGNYEPVDGLRLDSVMEVQKDFWPDWNSASGVYCVGEVDDGDPTFTCPYQKYLDGVLNYPTYFPLVRAFESSSGSISELYDMINEVKSDCVDSNLLGSFSENHDNPRFASYTSDFSLAKNALAFTILSDGIPIIYAGQEQHYDGGNNPYNREATWLSGYDTSAELYTFIAVLNQIRNYAIQWDNGYTTYKTYPIYQDDNNLAIRKGSDESQIITVLSNVGTDADSYSLTISGTGYTAGEVLTDLISCTNVTVDDSGDVSVTMTGGLPSVLYPTYKLVYDGHPC

SEQ ID NO: 73 - protein, 483 aa, Penicillium italicum
ATPAEWRSRSIYFMLTDRFARTDGSTTAACNTGDRKYCGGTWQGIIDKLDYIQGMGFTAIWITPVTSQLTVDTPYGEPYHGYWQQDIYALNSNYGTADDLKALAAALHERDMYLMVDVVANHMGYNGAGADVDYTKFNPFNDAKYFHPYCPITDYDNDTMAQNCWLGDDKVSLPDLDTQSMEVQDIWYDWVRSLVSNYSKTDEYKVDGLRIDTVKHVQKDFWPGYNKAAGVYCVGEVFDGDVDYTCPYQEVMDGVLNYPVYYPLLKAFQSTSGSMADLFNMINRVKSTCKDSTLLGNFLENHDNPRFASVTDDIALAKNAATFTIMADGIPIVYAGQEQHYSGGEDPANREALWLSGFNTDSELYKLIATVNGARNQAIAKSTNYTIYQNYPIYKDSSTIAMRKGYDSGQTITVLTNLGAGGKDYSVSIPDTGFAAGAKLTEVISCVSVTAGESGEVSVPMAGGAPRILLPTSLLEGSTLCSS